SEQUENCE LISTING (35 sequences)

SEQ ID NO: 1 - DNA, 3822 bp, Artificial Sequence
  Full length ORF (DNA template) encoding SARS-CoV2 spike protein (Esp3I linearized template)
atgttcgtgttcctggtgctgctgccgctggtgagcagccagtgcgtgaacctgacgacgcggacgcagctgccgccggcgtacacgaacagcttcacgcggggggtgtactacccggacaaggtgttccggagcagcgtgctgcacagcacgcaggacctgttcctgccgttcttcagcaacgtgacgtggttccacgcgatccacgtgagcgggacgaacgggacgaagcggttcgacaacccggtgctgccgttcaacgacggggtgtacttcgcgagcacggagaagagcaacatcatccgggggtggatcttcgggacgacgctggacagcaagacgcagagcctgctgatcgtgaacaacgcgacgaacgtggtgatcaaggtgtgcgagttccagttctgcaacgacccgttcctgggggtgtactaccacaagaacaacaagagctggatggagagcgagttccgggtgtacagcagcgcgaacaactgcacgttcgagtacgtgagccagccgttcctgatggacctggaggggaagcaggggaacttcaagaacctgcgggagttcgtgttcaagaacatcgacgggtacttcaagatctacagcaagcacacgccgatcaacctggtgcgggacctgccgcaggggttcagcgcgctggagccgctggtggacctgccgatcgggatcaacatcacgcggttccagacgctgctggcgctgcaccggagctacctgacgccgggggacagcagcagcgggtggacggcgggggcggcggcgtactacgtggggtacctgcagccgcggacgttcctgctgaagtacaacgagaacgggacgatcacggacgcggtggactgcgcgctggacccgctgagcgagaccaagtgcacgctgaagagcttcacggtggagaaggggatctaccagacgagcaacttccgggtgcagccgacggagagcatcgtgcggttcccgaacatcacgaacctgtgcccgttcggggaggtgttcaacgcgacgcggttcgcgagcgtgtacgcgtggaaccggaagcggatcagcaactgcgtggcggactacagcgtgctgtacaacagcgcgagcttcagcacgttcaagtgctacggggtgagcccgacgaagctgaacgacctgtgcttcacgaacgtgtacgcggacagcttcgtgatccggggggacgaggtgcggcagatcgcgccggggcagacggggaagatcgcggactacaactacaagctgccggacgacttcacggggtgcgtgatcgcgtggaacagcaacaacctggacagcaaggtgggggggaactacaactacctgtaccggctgttccggaagagcaacctgaagccgttcgagcgggacatcagcacggagatctaccaggcggggagcacgccgtgcaacggggtggaggggttcaactgctacttcccgctgcagagctacgggttccagccgacgaacggggtggggtaccagccgtaccgggtggtggtgctgagcttcgagctgctgcacgcgccggcgacggtgtgcgggccgaagaagagcacgaacctggtgaagaacaagtgcgtgaacttcaacttcaacgggctgacggggacgggggtgctgacggagagcaacaagaagttcctgccgttccagcagttcgggcgggacatcgcggacacgacggacgcggtgcgggacccgcagacgctggagatcctggacatcacgccgtgcagcttcgggggggtgagcgtgatcacgccggggacgaacacgagcaaccaggtggcggtgctgtaccaggacgtgaactgcacggaggtgccggtggcgatccacgcggaccagctgacgccgacgtggcgggtgtacagcacggggagcaacgtgttccagacgcgggcggggtgcctgatcggggcggagcacgtgaacaacagctacgagtgcgacatcccgatcggggcggggatctgcgcgagctaccagacgcagacgaacagcccgcggcgggcgcggagcgtggcgagccagagcatcatcgcgtacacgatgagcctgggggcggagaacagcgtggcgtacagcaacaacagcatcgcgatcccgacgaacttcacgatcagcgtgacgacggagatcctgccggtgagcatgacgaagacgagcgtggactgcacgatgtacatctgcggggacagcacggagtgcagcaacctgctgctgcagtacgggagcttctgcacgcagctgaaccgggcgctgacggggatcgcggtggagcaggacaagaacacgcaggaggtgttcgcgcaggtgaagcagatctacaagacgccgccgatcaaggacttcggggggttcaacttcagccagatcctgccggacccgagcaagccgagcaagcggagcttcatcgaggacctgctgttcaacaaggtgacgctggcggacgcggggttcatcaagcagtacggggactgcctgggggacatcgcggcgcgggacctgatctgcgcgcagaagttcaacgggctgacggtgctgccgccgctgctgacggacgagatgatcgcgcagtacacgagcgcgctgctggcggggacgatcacgagcgggtggacgttcggggcgggggcggcgctgcagatcccgttcgcgatgcagatggcgtaccggttcaacgggatcggggtgacgcagaacgtgctgtacgagaaccagaagctgatcgcgaaccagttcaacagcgcgatcgggaagatccaggacagcctgagcagcacggcgagcgcgctggggaagctgcaggacgtggtgaaccagaacgcgcaggcgctgaacacgctggtgaagcagctgagcagcaacttcggggcgatcagcagcgtgctgaacgacatcctgagccggctggacaaggtggaggcggaggtgcagatcgaccggctgatcacggggcggctgcagagcctgcagacgtacgtgacgcagcagctgatccgggcggcggagatccgggcgagcgcgaacctggcggcgacgaagatgagcgagtgcgtgctggggcagagcaagcgggtggacttctgcgggaaggggtaccacctgatgagcttcccgcagagcgcgccgcacggggtggtgttcctgcacgtgacgtacgtgccggcgcaggagaagaacttcacgacggcgccggcgatctgccacgacgggaaggcgcacttcccgcgggagggggtgttcgtgagcaacgggacgcactggttcgtgacgcagcggaacttctacgagccgcagatcatcacgacggacaacacgttcgtgagcgggaactgcgacgtggtgatcgggatcgtgaacaacacggtgtacgacccgctgcagccggagctggacagcttcaaggaggagctggacaagtacttcaagaaccacacgagcccggacgtggacctgggggacatcagcgggatcaacgcgagcgtggtgaacatccagaaggagatcgaccggctgaacgaggtggcgaagaacctgaacgagagcctgatcgacctgcaggagctggggaagtacgagcagtacatcaagtggccgtggtacatctggctggggttcatcgcggggctgatcgcgatcgtgatggtgacgatcatgctgtgctgcatgacgagctgctgcagctgcctgaaggggtgctgcagctgcgggagctgctgcaagttcgacgaggacgacagcgagccggtgctgaagggggtgaagctgcactacacgtga

SEQ ID NO: 2 - RNA, 3822 nt, Artificial Sequence
  Full length ORF (RNA) encoding SARS-CoV2 spike protein (from Esp3I linearized template)
auguucguguuccuggugcugcugccgcuggugagcagccagugcgugaaccugacgacgcggacgcagcugccgccggcguacacgaacagcuucacgcgggggguguacuacccggacaagguguuccggagcagcgugcugcacagcacgcaggaccuguuccugccguucuucagcaacgugacgugguuccacgcgauccacgugagcgggacgaacgggacgaagcgguucgacaacccggugcugccguucaacgacgggguguacuucgcgagcacggagaagagcaacaucauccggggguggaucuucgggacgacgcuggacagcaagacgcagagccugcugaucgugaacaacgcgacgaacguggugaucaaggugugcgaguuccaguucugcaacgacccguuccuggggguguacuaccacaagaacaacaagagcuggauggagagcgaguuccggguguacagcagcgcgaacaacugcacguucgaguacgugagccagccguuccugauggaccuggaggggaagcaggggaacuucaagaaccugcgggaguucguguucaagaacaucgacggguacuucaagaucuacagcaagcacacgccgaucaaccuggugcgggaccugccgcagggguucagcgcgcuggagccgcugguggaccugccgaucgggaucaacaucacgcgguuccagacgcugcuggcgcugcaccggagcuaccugacgccgggggacagcagcagcggguggacggcgggggcggcggcguacuacgugggguaccugcagccgcggacguuccugcugaaguacaacgagaacgggacgaucacggacgcgguggacugcgcgcuggacccgcugagcgagaccaagugcacgcugaagagcuucacgguggagaaggggaucuaccagacgagcaacuuccgggugcagccgacggagagcaucgugcgguucccgaacaucacgaaccugugcccguucggggagguguucaacgcgacgcgguucgcgagcguguacgcguggaaccggaagcggaucagcaacugcguggcggacuacagcgugcuguacaacagcgcgagcuucagcacguucaagugcuacggggugagcccgacgaagcugaacgaccugugcuucacgaacguguacgcggacagcuucgugauccggggggacgaggugcggcagaucgcgccggggcagacggggaagaucgcggacuacaacuacaagcugccggacgacuucacggggugcgugaucgcguggaacagcaacaaccuggacagcaaggugggggggaacuacaacuaccuguaccggcuguuccggaagagcaaccugaagccguucgagcgggacaucagcacggagaucuaccaggcggggagcacgccgugcaacgggguggagggguucaacugcuacuucccgcugcagagcuacggguuccagccgacgaacggggugggguaccagccguaccgggugguggugcugagcuucgagcugcugcacgcgccggcgacggugugcgggccgaagaagagcacgaaccuggugaagaacaagugcgugaacuucaacuucaacgggcugacggggacgggggugcugacggagagcaacaagaaguuccugccguuccagcaguucgggcgggacaucgcggacacgacggacgcggugcgggacccgcagacgcuggagauccuggacaucacgccgugcagcuucgggggggugagcgugaucacgccggggacgaacacgagcaaccagguggcggugcuguaccaggacgugaacugcacggaggugccgguggcgauccacgcggaccagcugacgccgacguggcggguguacagcacggggagcaacguguuccagacgcgggcggggugccugaucggggcggagcacgugaacaacagcuacgagugcgacaucccgaucggggcggggaucugcgcgagcuaccagacgcagacgaacagcccgcggcgggcgcggagcguggcgagccagagcaucaucgcguacacgaugagccugggggcggagaacagcguggcguacagcaacaacagcaucgcgaucccgacgaacuucacgaucagcgugacgacggagauccugccggugagcaugacgaagacgagcguggacugcacgauguacaucugcggggacagcacggagugcagcaaccugcugcugcaguacgggagcuucugcacgcagcugaaccgggcgcugacggggaucgcgguggagcaggacaagaacacgcaggagguguucgcgcaggugaagcagaucuacaagacgccgccgaucaaggacuucgggggguucaacuucagccagauccugccggacccgagcaagccgagcaagcggagcuucaucgaggaccugcuguucaacaaggugacgcuggcggacgcgggguucaucaagcaguacggggacugccugggggacaucgcggcgcgggaccugaucugcgcgcagaaguucaacgggcugacggugcugccgccgcugcugacggacgagaugaucgcgcaguacacgagcgcgcugcuggcggggacgaucacgagcggguggacguucggggcgggggcggcgcugcagaucccguucgcgaugcagauggcguaccgguucaacgggaucggggugacgcagaacgugcuguacgagaaccagaagcugaucgcgaaccaguucaacagcgcgaucgggaagauccaggacagccugagcagcacggcgagcgcgcuggggaagcugcaggacguggugaaccagaacgcgcaggcgcugaacacgcuggugaagcagcugagcagcaacuucggggcgaucagcagcgugcugaacgacauccugagccggcuggacaagguggaggcggaggugcagaucgaccggcugaucacggggcggcugcagagccugcagacguacgugacgcagcagcugauccgggcggcggagauccgggcgagcgcgaaccuggcggcgacgaagaugagcgagugcgugcuggggcagagcaagcggguggacuucugcgggaagggguaccaccugaugagcuucccgcagagcgcgccgcacggggugguguuccugcacgugacguacgugccggcgcaggagaagaacuucacgacggcgccggcgaucugccacgacgggaaggcgcacuucccgcgggaggggguguucgugagcaacgggacgcacugguucgugacgcagcggaacuucuacgagccgcagaucaucacgacggacaacacguucgugagcgggaacugcgacguggugaucgggaucgugaacaacacgguguacgacccgcugcagccggagcuggacagcuucaaggaggagcuggacaaguacuucaagaaccacacgagcccggacguggaccugggggacaucagcgggaucaacgcgagcguggugaacauccagaaggagaucgaccggcugaacgagguggcgaagaaccugaacgagagccugaucgaccugcaggagcuggggaaguacgagcaguacaucaaguggccgugguacaucuggcugggguucaucgcggggcugaucgcgaucgugauggugacgaucaugcugugcugcaugacgagcugcugcagcugccugaaggggugcugcagcugcgggagcugcugcaaguucgacgaggacgacagcgagccggugcugaagggggugaagcugcacuacacguga

SEQ ID NO: 3 - DNA, 3822 bp, Artificial Sequence
  Full length ORF (DNA template) encoding SARS-CoV2 spike protein (BspQI linearized template)
atgttcgtgttcctggtgctgctgccgctggtgagcagccagtgcgtgaacctgacgacgcggacgcagctgccgccggcgtacacgaacagcttcacgcggggggtgtactacccggacaaggtgttccggagcagcgtgctgcacagcacgcaggacctgttcctgccgttcttcagcaacgtgacgtggttccacgcgatccacgtgagcgggacgaacgggacgaagcggttcgacaacccggtgctgccgttcaacgacggggtgtacttcgcgagcacggagaagagtaacatcatccgggggtggatcttcgggacgacgctggacagcaagacgcagagcctgctgatcgtgaacaacgcgacgaacgtggtgatcaaggtgtgcgagttccagttctgcaacgacccgttcctgggggtgtactaccacaagaacaacaagagctggatggagagcgagttccgggtgtacagcagcgcgaacaactgcacgttcgagtacgtgagccagccgttcctgatggacctggaggggaagcaggggaacttcaagaacctgcgggagttcgtgttcaagaacatcgacgggtacttcaagatctacagcaagcacacgccgatcaacctggtgcgggacctgccgcaggggttcagcgcgctggagccgctggtggacctgccgatcgggatcaacatcacgcggttccagacgctgctggcgctgcaccggagctacctgacgccgggggacagcagcagcgggtggacggcgggggcggcggcgtactacgtggggtacctgcagccgcggacgttcctgctgaagtacaacgagaacgggacgatcacggacgcggtggactgcgcgctggacccgctgagcgagaccaagtgcacgctgaagagtttcacggtggagaaggggatctaccagacgagcaacttccgggtgcagccgacggagagcatcgtgcggttcccgaacatcacgaacctgtgcccgttcggggaggtgttcaacgcgacgcggttcgcgagcgtgtacgcgtggaaccggaagcggatcagcaactgcgtggcggactacagcgtgctgtacaacagcgcgagcttcagcacgttcaagtgctacggggtgagcccgacgaagctgaacgacctgtgcttcacgaacgtgtacgcggacagcttcgtgatccggggggacgaggtgcggcagatcgcgccggggcagacggggaagatcgcggactacaactacaagctgccggacgacttcacggggtgcgtgatcgcgtggaacagcaacaacctggacagcaaggtgggggggaactacaactacctgtaccggctgttccggaagagtaacctgaagccgttcgagcgggacatcagcacggagatctaccaggcggggagcacgccgtgcaacggggtggaggggttcaactgctacttcccgctgcagagctacgggttccagccgacgaacggggtggggtaccagccgtaccgggtggtggtgctgagcttcgagctgctgcacgcgccggcgacggtgtgcgggccgaagaagagtacgaacctggtgaagaacaagtgcgtgaacttcaacttcaacgggctgacggggacgggggtgctgacggagagcaacaagaagttcctgccgttccagcagttcgggcgggacatcgcggacacgacggacgcggtgcgggacccgcagacgctggagatcctggacatcacgccgtgcagcttcgggggggtgagcgtgatcacgccggggacgaacacgagcaaccaggtggcggtgctgtaccaggacgtgaactgcacggaggtgccggtggcgatccacgcggaccagctgacgccgacgtggcgggtgtacagcacggggagcaacgtgttccagacgcgggcggggtgcctgatcggggcggagcacgtgaacaacagctacgagtgcgacatcccgatcggggcggggatctgcgcgagctaccagacgcagacgaacagcccgcggcgggcgcggagcgtggcgagccagagcatcatcgcgtacacgatgagcctgggggcggagaacagcgtggcgtacagcaacaacagcatcgcgatcccgacgaacttcacgatcagcgtgacgacggagatcctgccggtgagcatgacgaagacgagcgtggactgcacgatgtacatctgcggggacagcacggagtgcagcaacctgctgctgcagtacgggagcttctgcacgcagctgaaccgggcgctgacggggatcgcggtggagcaggacaagaacacgcaggaggtgttcgcgcaggtgaagcagatctacaagacgccgccgatcaaggacttcggggggttcaacttcagccagatcctgccggacccgagcaagccgagcaagcggagcttcatcgaggacctgctgttcaacaaggtgacgctggcggacgcggggttcatcaagcagtacggggactgcctgggggacatcgcggcgcgggacctgatctgcgcgcagaagttcaacgggctgacggtgctgccgccgctgctgacggacgagatgatcgcgcagtacacgagcgcgctgctggcggggacgatcacgagcgggtggacgttcggggcgggggcggcgctgcagatcccgttcgcgatgcagatggcgtaccggttcaacgggatcggggtgacgcagaacgtgctgtacgagaaccagaagctgatcgcgaaccagttcaacagcgcgatcgggaagatccaggacagcctgagcagcacggcgagcgcgctggggaagctgcaggacgtggtgaaccagaacgcgcaggcgctgaacacgctggtgaagcagctgagcagcaacttcggggcgatcagcagcgtgctgaacgacatcctgagccggctggacaaggtggaggcggaggtgcagatcgaccggctgatcacggggcggctgcagagcctgcagacgtacgtgacgcagcagctgatccgggcggcggagatccgggcgagcgcgaacctggcggcgacgaagatgagcgagtgcgtgctggggcagagcaagcgggtggacttctgcgggaaggggtaccacctgatgagcttcccgcagagcgcgccgcacggggtggtgttcctgcacgtgacgtacgtgccggcgcaggagaagaacttcacgacggcgccggcgatctgccacgacgggaaggcgcacttcccgcgggagggggtgttcgtgagcaacgggacgcactggttcgtgacgcagcggaacttctacgagccgcagatcatcacgacggacaacacgttcgtgagcgggaactgcgacgtggtgatcgggatcgtgaacaacacggtgtacgacccgctgcagccggagctggacagcttcaaggaggagctggacaagtacttcaagaaccacacgagcccggacgtggacctgggggacatcagcgggatcaacgcgagcgtggtgaacatccagaaggagatcgaccggctgaacgaggtggcgaagaacctgaacgagagcctgatcgacctgcaggagctggggaagtacgagcagtacatcaagtggccgtggtacatctggctggggttcatcgcggggctgatcgcgatcgtgatggtgacgatcatgctgtgctgcatgacgagctgctgcagctgcctgaaggggtgctgcagctgcgggagctgctgcaagttcgacgaggacgacagcgagccggtgctgaagggggtgaagctgcactacacgtga

SEQ ID NO: 4 - RNA, 3822 nt, Artificial Sequence
  Full length ORF (RNA) encoding SARS-CoV2 spike protein (BspQI linearized template)
auguucguguuccuggugcugcugccgcuggugagcagccagugcgugaaccugacgacgcggacgcagcugccgccggcguacacgaacagcuucacgcgggggguguacuacccggacaagguguuccggagcagcgugcugcacagcacgcaggaccuguuccugccguucuucagcaacgugacgugguuccacgcgauccacgugagcgggacgaacgggacgaagcgguucgacaacccggugcugccguucaacgacgggguguacuucgcgagcacggagaagaguaacaucauccggggguggaucuucgggacgacgcuggacagcaagacgcagagccugcugaucgugaacaacgcgacgaacguggugaucaaggugugcgaguuccaguucugcaacgacccguuccuggggguguacuaccacaagaacaacaagagcuggauggagagcgaguuccggguguacagcagcgcgaacaacugcacguucgaguacgugagccagccguuccugauggaccuggaggggaagcaggggaacuucaagaaccugcgggaguucguguucaagaacaucgacggguacuucaagaucuacagcaagcacacgccgaucaaccuggugcgggaccugccgcagggguucagcgcgcuggagccgcugguggaccugccgaucgggaucaacaucacgcgguuccagacgcugcuggcgcugcaccggagcuaccugacgccgggggacagcagcagcggguggacggcgggggcggcggcguacuacgugggguaccugcagccgcggacguuccugcugaaguacaacgagaacgggacgaucacggacgcgguggacugcgcgcuggacccgcugagcgagaccaagugcacgcugaagaguuucacgguggagaaggggaucuaccagacgagcaacuuccgggugcagccgacggagagcaucgugcgguucccgaacaucacgaaccugugcccguucggggagguguucaacgcgacgcgguucgcgagcguguacgcguggaaccggaagcggaucagcaacugcguggcggacuacagcgugcuguacaacagcgcgagcuucagcacguucaagugcuacggggugagcccgacgaagcugaacgaccugugcuucacgaacguguacgcggacagcuucgugauccggggggacgaggugcggcagaucgcgccggggcagacggggaagaucgcggacuacaacuacaagcugccggacgacuucacggggugcgugaucgcguggaacagcaacaaccuggacagcaaggugggggggaacuacaacuaccuguaccggcuguuccggaagaguaaccugaagccguucgagcgggacaucagcacggagaucuaccaggcggggagcacgccgugcaacgggguggagggguucaacugcuacuucccgcugcagagcuacggguuccagccgacgaacggggugggguaccagccguaccgggugguggugcugagcuucgagcugcugcacgcgccggcgacggugugcgggccgaagaagaguacgaaccuggugaagaacaagugcgugaacuucaacuucaacgggcugacggggacgggggugcugacggagagcaacaagaaguuccugccguuccagcaguucgggcgggacaucgcggacacgacggacgcggugcgggacccgcagacgcuggagauccuggacaucacgccgugcagcuucgggggggugagcgugaucacgccggggacgaacacgagcaaccagguggcggugcuguaccaggacgugaacugcacggaggugccgguggcgauccacgcggaccagcugacgccgacguggcggguguacagcacggggagcaacguguuccagacgcgggcggggugccugaucggggcggagcacgugaacaacagcuacgagugcgacaucccgaucggggcggggaucugcgcgagcuaccagacgcagacgaacagcccgcggcgggcgcggagcguggcgagccagagcaucaucgcguacacgaugagccugggggcggagaacagcguggcguacagcaacaacagcaucgcgaucccgacgaacuucacgaucagcgugacgacggagauccugccggugagcaugacgaagacgagcguggacugcacgauguacaucugcggggacagcacggagugcagcaaccugcugcugcaguacgggagcuucugcacgcagcugaaccgggcgcugacggggaucgcgguggagcaggacaagaacacgcaggagguguucgcgcaggugaagcagaucuacaagacgccgccgaucaaggacuucgggggguucaacuucagccagauccugccggacccgagcaagccgagcaagcggagcuucaucgaggaccugcuguucaacaaggugacgcuggcggacgcgggguucaucaagcaguacggggacugccugggggacaucgcggcgcgggaccugaucugcgcgcagaaguucaacgggcugacggugcugccgccgcugcugacggacgagaugaucgcgcaguacacgagcgcgcugcuggcggggacgaucacgagcggguggacguucggggcgggggcggcgcugcagaucccguucgcgaugcagauggcguaccgguucaacgggaucggggugacgcagaacgugcuguacgagaaccagaagcugaucgcgaaccaguucaacagcgcgaucgggaagauccaggacagccugagcagcacggcgagcgcgcuggggaagcugcaggacguggugaaccagaacgcgcaggcgcugaacacgcuggugaagcagcugagcagcaacuucggggcgaucagcagcgugcugaacgacauccugagccggcuggacaagguggaggcggaggugcagaucgaccggcugaucacggggcggcugcagagccugcagacguacgugacgcagcagcugauccgggcggcggagauccgggcgagcgcgaaccuggcggcgacgaagaugagcgagugcgugcuggggcagagcaagcggguggacuucugcgggaagggguaccaccugaugagcuucccgcagagcgcgccgcacggggugguguuccugcacgugacguacgugccggcgcaggagaagaacuucacgacggcgccggcgaucugccacgacgggaaggcgcacuucccgcgggaggggguguucgugagcaacgggacgcacugguucgugacgcagcggaacuucuacgagccgcagaucaucacgacggacaacacguucgugagcgggaacugcgacguggugaucgggaucgugaacaacacgguguacgacccgcugcagccggagcuggacagcuucaaggaggagcuggacaaguacuucaagaaccacacgagcccggacguggaccugggggacaucagcgggaucaacgcgagcguggugaacauccagaaggagaucgaccggcugaacgagguggcgaagaaccugaacgagagccugaucgaccugcaggagcuggggaaguacgagcaguacaucaaguggccgugguacaucuggcugggguucaucgcggggcugaucgcgaucgugauggugacgaucaugcugugcugcaugacgagcugcugcagcugccugaaggggugcugcagcugcgggagcugcugcaaguucgacgaggacgacagcgagccggugcugaagggggugaagcugcacuacacguga

SEQ ID NO: 5 - protein, 1273 aa, Artificial Sequence
  SARS-CoV2 spike protein amino acid sequence (GenBank: QHD43416.1)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - RNA, 4123 nt, Artificial Sequence
  mRNA sequence encoding SARS-CoV2 spike protein (Esp3I linearized template), and including non-coding regions (ITS, 5′ UTR, Kozak Sequence, 3′ UTR)
gaggagaccaggaauuacauuugcuucugacacaacuguguucacuagcaaccucaaacagagccgccauguucguguuccuggugcugcugccgcuggugagcagccagugcgugaaccugacgacgcggacgcagcugccgccggcguacacgaacagcuucacgcgggggguguacuacccggacaagguguuccggagcagcgugcugcacagcacgcaggaccuguuccugccguucuucagcaacgugacgugguuccacgcgauccacgugagcgggacgaacgggacgaagcgguucgacaacccggugcugccguucaacgacgggguguacuucgcgagcacggagaagagcaacaucauccggggguggaucuucgggacgacgcuggacagcaagacgcagagccugcugaucgugaacaacgcgacgaacguggugaucaaggugugcgaguuccaguucugcaacgacccguuccuggggguguacuaccacaagaacaacaagagcuggauggagagcgaguuccggguguacagcagcgcgaacaacugcacguucgaguacgugagccagccguuccugauggaccuggaggggaagcaggggaacuucaagaaccugcgggaguucguguucaagaacaucgacggguacuucaagaucuacagcaagcacacgccgaucaaccuggugcgggaccugccgcagggguucagcgcgcuggagccgcugguggaccugccgaucgggaucaacaucacgcgguuccagacgcugcuggcgcugcaccggagcuaccugacgccgggggacagcagcagcggguggacggcgggggcggcggcguacuacgugggguaccugcagccgcggacguuccugcugaaguacaacgagaacgggacgaucacggacgcgguggacugcgcgcuggacccgcugagcgagaccaagugcacgcugaagagcuucacgguggagaaggggaucuaccagacgagcaacuuccgggugcagccgacggagagcaucgugcgguucccgaacaucacgaaccugugcccguucggggagguguucaacgcgacgcgguucgcgagcguguacgcguggaaccggaagcggaucagcaacugcguggcggacuacagcgugcuguacaacagcgcgagcuucagcacguucaagugcuacggggugagcccgacgaagcugaacgaccugugcuucacgaacguguacgcggacagcuucgugauccggggggacgaggugcggcagaucgcgccggggcagacggggaagaucgcggacuacaacuacaagcugccggacgacuucacggggugcgugaucgcguggaacagcaacaaccuggacagcaaggugggggggaacuacaacuaccuguaccggcuguuccggaagagcaaccugaagccguucgagcgggacaucagcacggagaucuaccaggcggggagcacgccgugcaacgggguggagggguucaacugcuacuucccgcugcagagcuacggguuccagccgacgaacggggugggguaccagccguaccgggugguggugcugagcuucgagcugcugcacgcgccggcgacggugugcgggccgaagaagagcacgaaccuggugaagaacaagugcgugaacuucaacuucaacgggcugacggggacgggggugcugacggagagcaacaagaaguuccugccguuccagcaguucgggcgggacaucgcggacacgacggacgcggugcgggacccgcagacgcuggagauccuggacaucacgccgugcagcuucgggggggugagcgugaucacgccggggacgaacacgagcaaccagguggcggugcuguaccaggacgugaacugcacggaggugccgguggcgauccacgcggaccagcugacgccgacguggcggguguacagcacggggagcaacguguuccagacgcgggcggggugccugaucggggcggagcacgugaacaacagcuacgagugcgacaucccgaucggggcggggaucugcgcgagcuaccagacgcagacgaacagcccgcggcgggcgcggagcguggcgagccagagcaucaucgcguacacgaugagccugggggcggagaacagcguggcguacagcaacaacagcaucgcgaucccgacgaacuucacgaucagcgugacgacggagauccugccggugagcaugacgaagacgagcguggacugcacgauguacaucugcggggacagcacggagugcagcaaccugcugcugcaguacgggagcuucugcacgcagcugaaccgggcgcugacggggaucgcgguggagcaggacaagaacacgcaggagguguucgcgcaggugaagcagaucuacaagacgccgccgaucaaggacuucgggggguucaacuucagccagauccugccggacccgagcaagccgagcaagcggagcuucaucgaggaccugcuguucaacaaggugacgcuggcggacgcgggguucaucaagcaguacggggacugccugggggacaucgcggcgcgggaccugaucugcgcgcagaaguucaacgggcugacggugcugccgccgcugcugacggacgagaugaucgcgcaguacacgagcgcgcugcuggcggggacgaucacgagcggguggacguucggggcgggggcggcgcugcagaucccguucgcgaugcagauggcguaccgguucaacgggaucggggugacgcagaacgugcuguacgagaaccagaagcugaucgcgaaccaguucaacagcgcgaucgggaagauccaggacagccugagcagcacggcgagcgcgcuggggaagcugcaggacguggugaaccagaacgcgcaggcgcugaacacgcuggugaagcagcugagcagcaacuucggggcgaucagcagcgugcugaacgacauccugagccggcuggacaagguggaggcggaggugcagaucgaccggcugaucacggggcggcugcagagccugcagacguacgugacgcagcagcugauccgggcggcggagauccgggcgagcgcgaaccuggcggcgacgaagaugagcgagugcgugcuggggcagagcaagcggguggacuucugcgggaagggguaccaccugaugagcuucccgcagagcgcgccgcacggggugguguuccugcacgugacguacgugccggcgcaggagaagaacuucacgacggcgccggcgaucugccacgacgggaaggcgcacuucccgcgggaggggguguucgugagcaacgggacgcacugguucgugacgcagcggaacuucuacgagccgcagaucaucacgacggacaacacguucgugagcgggaacugcgacguggugaucgggaucgugaacaacacgguguacgacccgcugcagccggagcuggacagcuucaaggaggagcuggacaaguacuucaagaaccacacgagcccggacguggaccugggggacaucagcgggaucaacgcgagcguggugaacauccagaaggagaucgaccggcugaacgagguggcgaagaaccugaacgagagccugaucgaccugcaggagcuggggaaguacgagcaguacaucaaguggccgugguacaucuggcugggguucaucgcggggcugaucgcgaucgugauggugacgaucaugcugugcugcaugacgagcugcugcagcugccugaaggggugcugcagcugcgggagcugcugcaaguucgacgaggacgacagcgagccggugcugaagggggugaagcugcacuacacgugaagcucgcuuucuugcuguccaauuucuauuaaagguuccuuuguucccuaaguccaacuacuaaacugggggauauuaugaagggccuugagcaucuggauucugccuaauaagaaacauuuauugucauugcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - RNA, 4123 nt, Artificial Sequence
  mRNA sequence encoding SARS-CoV2 spike protein (BspQI linearized template), and including non-coding regions (ITS, 5′ UTR, Kozak sequence, 3′ UTR)
gaggagaccaggaauuacauuugcuucugacacaacuguguucacuagcaaccucaaacagagccgccauguucguguuccuggugcugcugccgcuggugagcagccagugcgugaaccugacgacgcggacgcagcugccgccggcguacacgaacagcuucacgcgggggguguacuacccggacaagguguuccggagcagcgugcugcacagcacgcaggaccuguuccugccguucuucagcaacgugacgugguuccacgcgauccacgugagcgggacgaacgggacgaagcgguucgacaacccggugcugccguucaacgacgggguguacuucgcgagcacggagaagaguaacaucauccggggguggaucuucgggacgacgcuggacagcaagacgcagagccugcugaucgugaacaacgcgacgaacguggugaucaaggugugcgaguuccaguucugcaacgacccguuccuggggguguacuaccacaagaacaacaagagcuggauggagagcgaguuccggguguacagcagcgcgaacaacugcacguucgaguacgugagccagccguuccugauggaccuggaggggaagcaggggaacuucaagaaccugcgggaguucguguucaagaacaucgacggguacuucaagaucuacagcaagcacacgccgaucaaccuggugcgggaccugccgcagggguucagcgcgcuggagccgcugguggaccugccgaucgggaucaacaucacgcgguuccagacgcugcuggcgcugcaccggagcuaccugacgccgggggacagcagcagcggguggacggcgggggcggcggcguacuacgugggguaccugcagccgcggacguuccugcugaaguacaacgagaacgggacgaucacggacgcgguggacugcgcgcuggacccgcugagcgagaccaagugcacgcugaagaguuucacgguggagaaggggaucuaccagacgagcaacuuccgggugcagccgacggagagcaucgugcgguucccgaacaucacgaaccugugcccguucggggagguguucaacgcgacgcgguucgcgagcguguacgcguggaaccggaagcggaucagcaacugcguggcggacuacagcgugcuguacaacagcgcgagcuucagcacguucaagugcuacggggugagcccgacgaagcugaacgaccugugcuucacgaacguguacgcggacagcuucgugauccggggggacgaggugcggcagaucgcgccggggcagacggggaagaucgcggacuacaacuacaagcugccggacgacuucacggggugcgugaucgcguggaacagcaacaaccuggacagcaaggugggggggaacuacaacuaccuguaccggcuguuccggaagaguaaccugaagccguucgagcgggacaucagcacggagaucuaccaggcggggagcacgccgugcaacgggguggagggguucaacugcuacuucccgcugcagagcuacggguuccagccgacgaacggggugggguaccagccguaccgggugguggugcugagcuucgagcugcugcacgcgccggcgacggugugcgggccgaagaagaguacgaaccuggugaagaacaagugcgugaacuucaacuucaacgggcugacggggacgggggugcugacggagagcaacaagaaguuccugccguuccagcaguucgggcgggacaucgcggacacgacggacgcggugcgggacccgcagacgcuggagauccuggacaucacgccgugcagcuucgggggggugagcgugaucacgccggggacgaacacgagcaaccagguggcggugcuguaccaggacgugaacugcacggaggugccgguggcgauccacgcggaccagcugacgccgacguggcggguguacagcacggggagcaacguguuccagacgcgggcggggugccugaucggggcggagcacgugaacaacagcuacgagugcgacaucccgaucggggcggggaucugcgcgagcuaccagacgcagacgaacagcccgcggcgggcgcggagcguggcgagccagagcaucaucgcguacacgaugagccugggggcggagaacagcguggcguacagcaacaacagcaucgcgaucccgacgaacuucacgaucagcgugacgacggagauccugccggugagcaugacgaagacgagcguggacugcacgauguacaucugcggggacagcacggagugcagcaaccugcugcugcaguacgggagcuucugcacgcagcugaaccgggcgcugacggggaucgcgguggagcaggacaagaacacgcaggagguguucgcgcaggugaagcagaucuacaagacgccgccgaucaaggacuucgggggguucaacuucagccagauccugccggacccgagcaagccgagcaagcggagcuucaucgaggaccugcuguucaacaaggugacgcuggcggacgcgggguucaucaagcaguacggggacugccugggggacaucgcggcgcgggaccugaucugcgcgcagaaguucaacgggcugacggugcugccgccgcugcugacggacgagaugaucgcgcaguacacgagcgcgcugcuggcggggacgaucacgagcggguggacguucggggcgggggcggcgcugcagaucccguucgcgaugcagauggcguaccgguucaacgggaucggggugacgcagaacgugcuguacgagaaccagaagcugaucgcgaaccaguucaacagcgcgaucgggaagauccaggacagccugagcagcacggcgagcgcgcuggggaagcugcaggacguggugaaccagaacgcgcaggcgcugaacacgcuggugaagcagcugagcagcaacuucggggcgaucagcagcgugcugaacgacauccugagccggcuggacaagguggaggcggaggugcagaucgaccggcugaucacggggcggcugcagagccugcagacguacgugacgcagcagcugauccgggcggcggagauccgggcgagcgcgaaccuggcggcgacgaagaugagcgagugcgugcuggggcagagcaagcggguggacuucugcgggaagggguaccaccugaugagcuucccgcagagcgcgccgcacggggugguguuccugcacgugacguacgugccggcgcaggagaagaacuucacgacggcgccggcgaucugccacgacgggaaggcgcacuucccgcgggaggggguguucgugagcaacgggacgcacugguucgugacgcagcggaacuucuacgagccgcagaucaucacgacggacaacacguucgugagcgggaacugcgacguggugaucgggaucgugaacaacacgguguacgacccgcugcagccggagcuggacagcuucaaggaggagcuggacaaguacuucaagaaccacacgagcccggacguggaccugggggacaucagcgggaucaacgcgagcguggugaacauccagaaggagaucgaccggcugaacgagguggcgaagaaccugaacgagagccugaucgaccugcaggagcuggggaaguacgagcaguacaucaaguggccgugguacaucuggcugggguucaucgcggggcugaucgcgaucgugauggugacgaucaugcugugcugcaugacgagcugcugcagcugccugaaggggugcugcagcugcgggagcugcugcaaguucgacgaggacgacagcgagccggugcugaagggggugaagcugcacuacacgugaagcucgcuuucuugcuguccaauuucuauuaaagguuccuuuguucccuaaguccaacuacuaaacugggggauauuaugaagggccuugagcaucuggauucugccuaauaagaaacauuuauugucauugcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 6 bp, Artificial Sequence
  Initial Transcribed Sequence (ITS)
gggaga

SEQ ID NO: 9 - DNA, 6 bp, Artificial Sequence
  Initial Transcribed Sequence (ITS)
aggaga

SEQ ID NO: 10 - RNA, 15 nt, Artificial Sequence
  Initial Transcribed Sequence (ITS)
gggagaccaggaauu

SEQ ID NO: 11 - RNA, 15 nt, Artificial Sequence
  Initial Transcribed Sequence (ITS)
aggagaccaggaauu

SEQ ID NO: 12 - RNA, 46 nt, Artificial Sequence
  5′ untranslated region (UTR)
acauuugcuucugacacaacuguguucacuagcaaccucaaacaga

SEQ ID NO: 13 - DNA, 6 bp, Artificial Sequence
  Kozak sequence
gccgcc

SEQ ID NO: 14 - RNA, 133 nt, Artificial Sequence
  3′ untranslated region
agcucgcuuucuugcuguccaauuucuauuaaagguuccuuuguucccuaaguccaacuacuaaacugggggauauuaugaagggccuugagcaucuggauucugccuaauaagaaacauuuauugucauugc

SEQ ID NO: 15 - DNA, 100 bp, Artificial Sequence
  3′ poly(A) sequence
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - skipped

SEQ ID NO: 17 - RNA, 67 nt, Artificial Sequence
  ITS (SEQ ID: NO: 11) + HBG 5′ UTR (SEQ ID NO: 12) + Kozak sequence (SEQ ID NO: 13)
aggagaccaggaauuacauuugcuucugacacaacuguguucacuagcaaccucaaacagagccgcc

SEQ ID NO: 18 - RNA, 51 nt, Artificial Sequence
  5′ HBA sequence
agggcgaacuaguacucuucugguccccacagacucagagagaacgccacc

SEQ ID NO: 19 - RNA, 71 nt, Artificial Sequence
  5′ HSD sequence
agggucccgcagucggcguccagcggcucugcuuguucgugugugugucguugcaggccuuauucgccgcc

SEQ ID NO: 20 - RNA, 48 nt, Artificial Sequence
  5′ MRNA sequence
agggaaauaagagagaaaagaagaguaagaagaaauauaagagccacc

SEQ ID NO: 21 - RNA, 71 nt, Artificial Sequence
  5′ NCA-7d sequence
aggcaaaaaucaaaaucaaucaucaucacaacaucaacaaucaaucaucaacacaucaucaagacaccacc

SEQ ID NO: 22 - RNA, 133 nt, Artificial Sequence; identical to SEQ ID NO: 14
  3′ HBG sequence

SEQ ID NO: 23 - RNA, 278 nt, Artificial Sequence
  3′ AES-mtRNR1 sequence
cugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacc

SEQ ID NO: 24 - RNA, 186 nt, Artificial Sequence
  3′ ALB sequence
caucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuugucucugugcuccacucaccaaaaaauggaaagaaccu

SEQ ID NO: 25 - RNA, 110 nt, Artificial Sequence
  3′ HBA sequence
ugauaauaggcuggagccucgguggccaugcuucuugccccuugggccuccccccagccccuccuccccuuccugcacccguacccccguggucuuugaauaaagucuga

SEQ ID NO: 26 - RNA, 122 nt, Artificial Sequence
  3′ S27a+R3U sequence
uuguguaugcguuaauaaaaagaaggaacucguaaaaacucaauguauuucugaggaagcguggugcauaaugccacgcagcgucugcauaacuuuuauuauuucuuuuauuaaucaacaaa

SEQ ID NO: 27 - RNA, 67 nt, Artificial Sequence; identical to SEQ ID NO: 17
  5′ HBG-ITS sequence

SEQ ID NO: 28 - RNA, 67 nt, Artificial Sequence
  5′ HBG-ITS sequence
gggagaccaggaauuacauuugcuucugacacaacuguguucacuagcaaccucaaacagagccgcc

SEQ ID NO: 29 - RNA, 55 nt, Artificial Sequence
  5′ HBG sequence
aggacauuugcuucugacacaacuguguucacuagcaaccucaaacagagccgcc

SEQ ID NO: 30 - RNA, 53 nt, Artificial Sequence
  5′ HBG sequence
gacauuugcuucugacacaacuguguucacuagcaaccucaaacagagccgcc

SEQ ID NO: 31 - RNA, 85 nt, Artificial Sequence
  5′ NCA-7d-ITS sequence
aggagaccaggaauuggcaaaaaucaaaaucaaucaucaucacaacaucaacaaucaaucaucaacacaucaucaagacaccacc

SEQ ID NO: 32 - RNA, 85 nt, Artificial Sequence
  5′ NCA-7d-ITS sequence
gggagaccaggaauuggcaaaaaucaaaaucaaucaucaucacaacaucaacaaucaaucaucaacacaucaucaagacaccacc

SEQ ID NO: 33 - RNA, 71 nt, Artificial Sequence; identical to SEQ ID NO: 21
  5′ NCA-7d sequence

SEQ ID NO: 34 - RNA, 69 nt, Artificial Sequence
  5′ NCA-7d sequence
ggcaaaaaucaaaaucaaucaucaucacaacaucaacaaucaaucaucaacacaucaucaagacaccac

SEQ ID NO: 35 - RNA, 68 nt, Artificial Sequence
  5′ NCA-7d sequence
gcaaaaaucaaaaucaaucaucaucacaacaucaacaaucaaucaucaacacaucaucaagacaccac

(19) United States
(12) Patent Application Publication
(10) Pub. No.: US 2022/0370599 A1
(43) Pub. Date: Nov. 24, 2022

(54) MESSENGER RNA THERAPEUTICS AND COMPOSITIONS

(71) Applicant: Greenlight Biosciences, Inc., Medford, MA (US)

(72) Inventors: James Robbins ABSHIRE, Cambridge, MA (US); Christopher J.H. DAVITT, Jamaica Plain, MA (US); Ian HILL, Malden, MA (US); Lorenzo AULISA, Chesterfield, MO (US); Marcelo SAMSA, Hanover, MD (US); Nabanita DE, Lexington, MA (US); Michael HUDSON, Medford, MA (US); Rachit JAIN, Medford, MA (US); Himanshu DHAMANKAR, Arlington, MA (US); William FARMER, Concord, MA (US); Christopher GREGG, Melrose, MA (US)

(21) Appl. No.: 17/734,703

(22) Filed: May 2, 2022

Related U.S. Application Data
(60) Provisional application No. 63/253,481, filed on Oct. 7, 2021.
(60) Provisional application No. 63/182,290, filed on Apr. 30, 2021.

(51) Int. Cl. A61K 39/215; C07K 14/005

(52) U.S. Cl. CPC A61K 39/215; C07K 14/005; C12N 2770/20022; C12N 2770/20034; A61K 2039/53

(57) ABSTRACT

In the various aspects and embodiments, this disclosure provides messenger RNA (mRNA) constructs for therapeutic delivery, as well as methods for making such mRNA constructs and pharmaceutical compositions comprising the same (including mRNA vaccine compositions). In still other aspects, the invention provides methods for treating patients by expression of therapeutic proteins, including for preventing or reducing probability of infection by, or illness involving, a virus. Exemplary viruses include coronaviruses (such as SARS-CoV-2 and variants therefore) and influenza viruses, among others.

This application claims the benefit of and priority to U.S. Provisional Application Nos. 63/182,290 filed Apr. 30, 2021, and 63/253,481 filed Oct. 7, 2021, the contents of which are hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, filed Jul. 18, 2022 is named “GLB-001_128041-5001_SequenceListing_ST25.txt” and is 53,348 bytes in size.

### BACKGROUND

mRNA therapeutics, including mRNA vaccines, are promising approaches to the treatment or prevention of disease. However, production of the mRNA at scale and introducing the mRNA into cells so as to express the protein of interest at therapeutically effective levels are key challenges. In the various aspects and embodiments, the present disclosure meets these objectives.

### DESCRIPTION OF THE FIGURES

FIG. 1 illustrates a DNA construct for in vitro or cell-free synthesis of mRNA encoding the SARS-CoV-2 spike protein and the resultant mRNA. In some embodiments, mRNAs are synthesized using unmodified (“canonical”) nucleotides, or optionally with uridine nucleotides replaced by modified uridine, such as pseudouridine (ψ). In some embodiments, the sequence encoding SARS-CoV-2 spike protein can be replaced with a sequence encoding any given therapeutic or antigenic protein.

FIGS. 2A-2C show biochemical characterization of unformulated GLB-COV2-042 (SJ3) and GLB-COV2-043 (SJ2) mRNAs, compared to the firefly luciferase (SJ1) control RNA. (FIG. 2A) Purity assessment by capillary electrophoresis of purified RNAs. (FIG. 2B) Size determination of SJ2 and SJ3 by denaturing agarose gel electrophoresis. (FIG. 2C) dsRNA assessment by J2 immunoblot.

FIGS. 3A-3D demonstrate spike protein expression from the mRNA constructs: (FIG. 3A) A schematic of the spike protein domains; (FIG. 3B) A Western blot of spike protein expressed in 293T cells by transfection of RNA constructs; (FIG. 3C) Standard curve for quantifying spike protein expression by enzyme-linked immunosorbent assay (ELISA), prepared using recombinant spike protein; and (FIG. 3D) Quantification of spike protein expression by ELISA.

FIG. 4A and FIG. 4B show that mRNA produced by cell-free reactions and capped using CleanCap™ AG and ITS of SEQ ID NO: 11 achieved similar titers as reactions producing uncapped RNA (having an ITS of SEQ ID NO: 10) (FIG. 4A). Analysis by Bioanalyzer demonstrated RNA products from both reactions were of the expected size and similar purity (FIG. 4B). The mRNA tested encodes SARS-CoV-2 (Wuhan) spike.

FIG. 5A and FIG. 5B show that mRNA produced by cell-free reactions producing capped RNA using CleanCap™ AG and the ITS of SEQ ID NO: 11 produced consistent titers across multiple open reading frame sequences including the substitution of pseudouridine for uridine (FIG. 5A). RNA products of these reactions migrated at the expected sizes. All molecules were produced with similarly high purity (FIG. 5B). The mRNAs encode as follows: molecule 1, firefly luciferase; molecule 2, SARS-CoV-2 (Wuhan) spike; molecule 3: hemagglutinin (HA) from influenza A/California/07/2009 (H1N1); and molecule 4: neuraminidase (NA) from influenza A/California/07/2009 (H1N1).

FIG. 6 illustrates spike protein mutations in various SARS-CoV-2 variants.

FIG. 7 illustrates spike protein mutations in various SARS-CoV-2 variants.

FIG. 8A and FIG. 8B show FRNT results on days 21 and 39 respectively, in a study in which golden Syrian hamsters were vaccinated with 100, 30 or 5 μg of GLB-COV-2-042 or GLB-COV-2-043 on days 0 and 21. Means and standard deviations are shown. Data from controls were combined for statistical analyses. Statistical analyses were performed using rank-based Mann-Whitney and Holm-Šid\acute{α}ck multiple comparisons tests. Statistical signifiers above bars represent significance between that group and controls. *=p<0.05; ***=p<0.005; ****=p<0.001.

FIG. 9 depicts percent body weight change during 14 days interval post infection for golden Syrian hamsters vaccinated with 100, 30, or 5 μg of GLB-COV2-042 or GLB-COV2-043 on days 0 and 21 and challenged on day 42.

FIGS. 10A-10D depict a quantification of SARs-CoV-2 in lungs of vaccinated hamsters (100, 30, or 5 μg of GLB-COV2-042 or GLB-COV2-043 on days 0 and 21) following viral challenge on day 42. FIG. 10A and FIG. 10B show amount of SARS-CoV-2 nucleocapsid detected via RT-qPCR at days 2 and 4, respectively, post challenge. FIG. 10C and FIG. 10D show amount of active viral particles via TCID₅₀ at days 2 and 4, respectively, post challenge. Means and standard deviations are shown. Data from controls were combined for statistical analysis. Statistical analyses were performed using rank-based Mann-Whitney and Holm-Šid\acute{α}ck multiple comparisons tests. Statistical signifiers above bars represent significance between that group and the controls. *=p<0.05; ***=p<0.005; **** =p<0.001.

FIGS. 11A-11D depict a quantification of SARS-CoV-2 in the nasopharynx of vaccinated hamsters (100, 30, or 5 μg of GLB-COV2-042 or GLB-COV2-043 on days 0 and 21) following viral challenge on day 42. FIG. 11A and FIG. 11B SARS-CoV-2 nucleocapsid detected via qPCR at days 2 and 4, respectively, post challenge. FIG. 11C and FIG. 11D show amount of active viral particles via TCID₅₀ at days 2 and 4, respectively, post challenge. Means and standard deviations are shown. Data from controls were combined for statistical analysis. Statistical analyses were performed using rank-based Mann-Whitney and Holm-Šid\acute{α}ck multiple comparisons tests. Statistical signifiers above bars represent significance between that group and the controls. *=p<0.05; ***=p<0.005; **** =p<0.001.

FIGS. 12A and 12B show neutralizing antibody levels in sera samples isolated from mice immunized with mRNA vaccines of the present disclosure in a pseudovirus neutralization assay measuring functional neutralizing antibody responses against the Wuhan (wildtype) SARS-CoV-2 and variants of concern Beta (FIG. 12A) and Delta (FIG. 12B).

FIG. 13 shows neutralizing antibody levels in sera samples isolated from mice immunized with mRNA vaccines of the present disclosure in a pseudovirus neutralization assay measuring functional neutralizing antibody responses against the Wuhan (wildtype) SARS-CoV-2.

FIGS. 14A-14C show that all vaccine candidates wildtype (FIG. 14A), Beta (FIG. 14B), and Delta (FIG. 14C) were biased toward the desirable Th1 T cell responses at the studied mRNA doses 1 μg and 10 μg when compared to the saline group. FIGS. 14A, 14B, and 14C show CD4 response to stimulation.

FIGS. 15A-15C show that all vaccine candidates wildtype (FIG. 15A), Beta (FIG. 15B), and Delta (FIG. 15C) were biased toward the desirable Th1 T cell responses at the studied mRNA doses 1 μg and 10 μg when compared to the saline group. FIGS. 15A, 15B, and 15C show CD8 response to stimulation.

FIG. 16 shows the effect of a vaccine booster shot (third shot of an mRNA SARS-CoV-2 candidate) and the longitudinal neutralizing antibody levels in sera, blood samples isolated from wild type C57BL/6 mice, at several time points, immunized with the GLB mRNA vaccine candidate GLB-COV-2-043.

FIG. 17 shows that mRNA synthesis titer is affected by 5′ sequence, where the ITS of the present disclosure sequence yields the highest titers of mRNA among the sequences tested.

FIGS. 18A and 18B show that the ITS of the present disclosure increases in vitro transcription (IVT) synthesis yield in different 5′ UTR and coding sequence contexts of GFP (FIG. 18A) and SARS-CoV-2 S (FIG. 18B).

FIGS. 19A and 19B show that the ITS of the present disclosure increases CFR synthesis yield in different 5′ UTR and coding sequence contexts of GFP (FIG. 19A) and SARS-CoV-2 S (FIG. 19B).

FIGS. 20A and 20B show that the ITS of the present disclosure added to HBG and NCA 5′ UTRs maintains translational potency, with slight enhancement of gene expression observed with the 5′ ITS-HBG-Kozak in GFP context (FIG. 20A) compared to SARS-CoV-2 S (FIG. 20B). Results show a statistically significant increase in expression of GFP in both the -ITS-HBG-Kozak and ITS-NCA7d-Kozak vs. their respective versions lacking the ITS (FIG. 20A).

### DETAILED DESCRIPTION

In the various aspects and embodiments, this disclosure provides messenger RNA (mRNA) constructs for therapeutic delivery, as well as methods for making such mRNA constructs and pharmaceutical compositions comprising the same (including mRNA vaccine compositions). In still other aspects, the present disclosure provides methods for treating patients by expression of therapeutic proteins, including for preventing or reducing probability of infection by, or illness involving, a virus. Exemplary viruses include coronaviruses (such as SARS-CoV-2 and variants therefore) influenza viruses, and herpes viruses, among others.

In some aspects and embodiments, the disclosure provides an mRNA encoding a SARS-CoV-2 wild-type spike protein. In some embodiments, the mRNA comprises an Initial Transcribed Sequence (ITS) of 6 to 20 nucleotides, and which may comprise the nucleotide sequence of SEQ ID NO: 8, SEQ ID NO: 9, SEQ ID NO: 10, or SEQ ID NO: 11, or a derivative thereof as described herein, and which optionally comprises one or more modified bases. Alternatively or in addition, the mRNA encodes the SARS-CoV-2 spike protein in a prefusion stabilized state, as described herein.

Severe acute respiratory syndrome coronavirus 2 (SARS-CoV-2) is a novel (3-coronavirus first identified in 2019. SARS-CoV-2 is a single-stranded RNA-enveloped virus encoding structural and nonstructural proteins. The S, E, M, and N genes encode structural proteins, whereas nonstructural proteins include 3-chymotrypsin-like protease, papain-like protease, and RNA-dependent RNA polymerase.

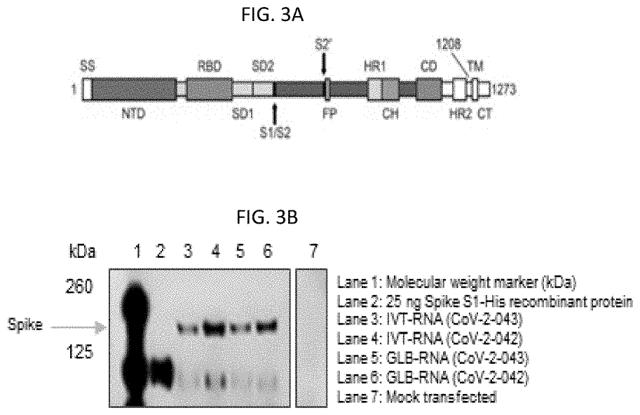

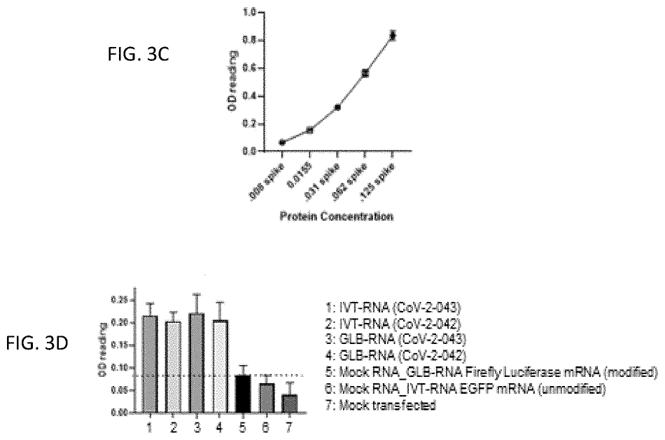

The spike (S) protein of SARS-CoV-2 is involved in receptor recognition and cell membrane fusion. The S protein is 180-200 kDa in size and contains an extracellular N-terminus, a transmembrane (TM) domain anchored in the viral membrane, and a small intracellular C-terminal domain. See FIG. 3A. The S protein exists in a prefusion conformation, and once the virus interacts with the host cell, structural rearrangement of the S protein occurs, allowing the virus to fuse with the host cell membrane. The S protein present in the virus envelope is coated with polysaccharide molecules evading surveillance of the host immune system. The S protein is composed of two subunits, S1 and S2. The S1 subunit contains a receptor-binding domain that binds to angiotensin-converting enzyme 2 on host cells, while the S2 subunit mediates viral cell membrane fusion. When the S protein binds to the receptor, a serine protease located on the host cell membrane activates the S protein (cleaving it into S1 and S2 subunits), which promotes virus entry into the cell. In accordance with embodiments of the present disclosure, the mRNA encodes the S protein in its uncleaved state, that is, comprising S1 and S2 subunits. That is, the mRNA encodes a wild type S protein.

In other embodiments, the mRNA may be a vaccine against other viruses, such as, for example, influenza viruses or herpes viruses. The composition may comprise a vaccine and the mRNA may encode a known antigen for any given virus. In some embodiments, the composition may comprise a shingles vaccine. In such embodiments, the mRNA may encode one or more known varicella antigens, such as glycoprotein E, glycoprotein B, glycoprotein H, glycoprotein L, and glycoprotein I. In other embodiments, the vaccine is an influenza vaccine with an mRNA encoding for one or more of hemagglutinin and/or neuraminidase.

The mRNA constructs in accordance with this disclosure can be designed to improve synthesis (e.g., improved yield using in vitro or cell-free RNA synthesis processes). In some embodiments, the mRNA is transcribed from a DNA template in vitro or in a cell-free system, e.g., using T7 RNA polymerase. In vitro transcription is well known in the art. In some embodiments, the mRNA is synthesized using a cell-free process as described in WO 2020/205793 or U.S. Pat. No. 10,858,385, which are hereby incorporated by reference in their entireties, or as described herein. For example, in vitro transcription or cell-free transcription processes will involve a DNA template having a promoter. The DNA template will comprise an open reading frame (“ORF”) encoding the protein of interest (e.g., S protein as described herein or an antigen for any given virus) and with untranslated regions. If positioned on the 5′ side of the ORF, the untranslated region is called a 5′ UTR. If positioned on the 3′ side of the ORF, the untranslated region is called a 3′ UTR.

A 5′ UTR provides sequences and secondary structures that regulate translation. A 5′ UTR advantageously comprises a sequence that is recognized by the ribosome that allows the ribosome to bind and initiate translation of the mRNA. Sequences and structures of 3′ UTRs can govern mRNA stability, for example. 3′ UTR regulatory elements are recognized by a wide variety of trans-acting factors that include microRNAs (miRNAs), their associated machinery, and RNA-binding proteins (RBPs). In turn, these factors instigate common mechanistic strategies to execute the regulatory programs that are encoded by 3′ UTRs. In some embodiments, the 5′ UTR and/or 3′ UTR can be substantially derived from the human α- and/or β-globin genes. Other suitable UTRs for therapeutic mRNAs are described herein and in Orlandini von Niessen, et al., Improving mRNA-Based Therapeutic Gene Delivery by Expression-Augmenting 3′ UTRs Identified by Cellular Library Screening, Molecular Therapy Vol. 27, No. 4 (2019); Trepotec Z., et al., Maximizing the Translational Yield of mRNA Therapeutics by Minimizing 5′-UTRs, Tissue Eng Part A (2019) January; 25(1-2):69-79.

In some embodiments the 5′ UTR comprises an initial transcribed sequence (ITS) positioned at the 5′ end of the 5′ UTR that improves the efficiency of transcription initiation to thereby maximize RNA product yield and minimize production of abortive transcription products from transcription reactions. An ITS is a short sequence of about 6 to 15 nucleotides that, when present, has a critical role in the early stages of transcription (initiation and the transition to elongation phase via promoter clearance), and influences the overall rate and yield of transcription from a given promoter. In some embodiments, the ITS is as described in WO 2020/205793 and/or WO 2021/113774, which disclosures are hereby incorporated by reference. For example, in some embodiments, an ITS is a naturally occurring ITS, or is a consensus ITS found downstream of a T7 class III promoter.

In some embodiments, the ITS is 6 to about 20 nucleotides in length, or is 6 to about 15 nucleotides in length, and is otherwise heterologous to the 5′ UTR sequence. The ITS may comprise the 5′ sequence GGGAGA (SEQ ID NO: 8) or AGGAGA (SEQ ID NO: 9). In some embodiments, the ITS comprises at least 6, at least 8, at least 10, or at least 12 consecutive nucleotides from the 5′ end of the following sequence: GGGAGACCAGGAAUU (SEQ ID NO: 10) or AGGAGACCAGGAAUU (SEQ ID NO: 11). In some embodiments, the ITS is a modified version of SEQ ID NO: 10 or SEQ ID NO: 11, for example having one, two, three, four, or five nucleotide changes. In some embodiments, the ITS consists of the nucleotide sequence of SEQ ID NO: 10 or SEQ ID NO: 11 followed by further 5′ UTR sequences (e.g., sequences from a mammalian or human mRNA 5′ UTR, including a mammalian or human 5′ UTR disclosed herein).

For simplicity nucleotide sequences may be shown herein using DNA nucleotide sequences (i.e., including T nucleobases) or as RNA nucleotide sequences (i.e., including U nucleobases). It is understood from the context that when the sequence is intended to be RNA, T nucleotides are substituted as U (or modified U as described herein), and vice versa.

In some embodiments, the ITS comprises the nucleotide sequence of SEQ ID NO: 8, SEQ ID NO: 9, SEQ ID NO: 10, or SEQ ID NO: 11 (or a portion thereof as described above) and optionally comprising one or more nucleotide substitutions. Where the in vitro transcription reaction includes modified bases (such as modified uridine for uridine), the ITS will contain such modified bases. Nucleotide substitutions can be selected from those that do not negatively impact or which improve transcription yield from a T7 class III promoter. In various embodiments, the ITS, such as the ITS of SEQ ID NO: 8, SEQ ID NO: 9, SEQ ID NO: 10, or SEQ ID NO: 11 (or portions thereof) may have one or more modified bases. Modified bases include those described in U.S. Pat. No. 8,691,966, which is hereby incorporated by reference in its entirety. Synthetic RNA comprising only canonical nucleotides (i.e., G, C, A, and U) can bind to pattern recognition receptors and induce a cellular response. This response can result in translation block, the secretion of inflammatory cytokines, and cell death. RNA comprising certain non-canonical nucleotides can evade detection by this innate immune system and can be translated at high efficiency into protein. In addition, in accordance with the present disclosure, such modified bases do not impact efficiency of transcription, and thus do not negatively impact RNA yield.

In some embodiments, where modified uridine is used with the in vitro transcription reaction, the ITS will comprise modified uridine. For example, at least about 50% or all uridines can be modified uridines, such as pseudouridine, Nl-methyl-pseudouridine, and/or 5-methoxy-uridine. In some embodiments, uridines of the ITS sequence are replaced with pseudouridine or N1-methyl-pseudouridine. Other modified bases for use with the ITS include 5-methyl cytidine.

In various embodiments, the mRNA includes one or more modified nucleotides, such as one or more of: 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-ydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine,pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-az acytidine, 5-hydroxy-5-azacytidine, 5-methylp s eudo iso cytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine,N4-hydroxypseudoisocytidine, N4-hydroxy-5-methylcytidine, N4-hydroxy-5-amino cytidine,N4-hydroxy-5-hydroxycytidine, N4-hydroxy-5-methyl-5-azacytidine, N4-hydroxy-5-amino-5-azacytidine, N4-hydroxy-5-hydroxy-5-az acytidine, N4-hydroxy-5-methylp seudo is o cytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminop seudo is o cytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino -5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, 6-thio-7-deaza-8-azaguanosin, and N1-methylpseudouridine.

In some embodiments, the ITS of the mRNA does not contain any modified bases, that is, all bases are canonical nucleotides. As used herein, the term “canonical” nucleotides in the context of mRNA includes adenine, guanine, cytosine, and uracil bases.

In various embodiments, the mRNA comprises a 5′ cap. An mRNA cap serves a variety of functions, including, but not limited to, recruiting ribosomal subunits, promoting ribosome assembly and translation, and protecting the mRNA from exonuclease activity. Capping can be achieved using a variety of methods. In some embodiments, capping is achieved using one or more enzymes. The process of capping can involve a variety of enzymatic activities, such as RNA 5-triphosphatase activity, guanylyltransferase activity, guanylyl methyltransferase activity, and 2′-O-Methyltransferase activity. In some embodiments, one protein or complex accomplishes all four functions. In some embodiments, the four activities are accomplished by two, three, or four enzymes.

Capping can be performed at a variety of different steps of the mRNA synthesis process. Capping can occur co-transcriptionally or post-transcriptionally. For example, the cap can be added after RNA synthesis, and before or after an enzymatic polyadenylation step, if enzymatic polyadenylation is performed. In some embodiments, the RNA is capped in the reaction mix before purification. In other embodiments, the RNA is capped after it is purified.

In some embodiments, mRNA is capped using a cap analog. Cap analogs can include dinucleotide cap analogs (e.g., standard cap analog or anti-reverse cap analog) or 3+ nucleotide cap analogs (e.g., CleanCap™ from TriLink BioTechnologies, Inc., San Diego, Calif.). In some embodiments, the 5′ cap is m7G (cap 0). The m7G cap structure is a 7-methylguansine triphosphate linked to the 5′ end of the mRNA via a 5′→5′ triphosphate linkage. In some embodiments, the cap structure can be further modified by adding a methyl group to the 2′O position of the initiating nucleotide of the mRNA (cap 1). In some embodiments, the cap structure can be further modified by adding methyl group(s) to the 2′O position of subsequent nucleotides of the mRNA (hypermethylated caps such as cap 2, 3, 4, etc.).

In some embodiments, capping enzymes are added to the mRNA synthesis reaction (described below), along with a methyl donor (e.g., S-adenosylmethionine), and either GTP or GMP with polyphosphate. In some embodiments, GMP is converted to GTP by kinases present in the cell-free reaction. A non-comprehensive list of enzymes for potential use in capping messenger RNAs are described in WO 2020/205793, which is hereby incorporated by reference. In some embodiments, capping can be performed after the RNA polymerization step.

In various embodiments, the mRNA, in addition to an ORF encoding the protein of interest, further comprises a 5′ UTR (e.g., including an ITS and Kozak sequence), a 3′ UTR, and a PolyA tract. In some embodiments, the 5′ UTR comprises the nucleotide sequence substantially of SEQ ID NO: 12 or a derivative thereof. Derivatives of the sequence of SEQ ID NO: 12 can have up to about 20%, up to about 15%, up to about 10%, or up to about 5% of nucleotides substituted. Such substitutions include those that do not negatively impact, and/or those that enhance, transcription of the mRNA in the in vitro or cell-free system, and/or do not negatively impact translation and/or stability in host cells, which can be evaluated using in vitro cell lines that are representative of the target cell or tissue. The 5′ UTR comprises a Kozak sequence, which optionally has the sequence of SEQ ID NO: 13 (optionally with modified bases as described herein). In some embodiments, the 3′ UTR comprises the nucleotide sequence of SEQ ID NO: 14 or a derivative thereof. Derivatives of the sequence of SEQ ID NO: 14 can have up to about 20%, up to about 15%, up to about 10%, or up to about 5% of nucleotides substituted. Such substitutions include those that do not negatively impact, and/or can include those that enhance, transcription of the mRNA in the in vitro or cell-free system, and/or do not negatively impact translation and/or stability in host cells, which can be evaluated using in vitro cell lines that are representative of the target cell or tissue.

In some embodiments that employ modified bases during the in vitro transcription reaction, the 5′ UTR and 3′ UTR will have modified bases (e.g., such as modified bases include those described in U.S. Pat. No. 8,691,966, which are hereby incorporated by reference in its entirety). In some embodiments, the 5′ UTR and 3′ UTR comprise modified uridine. For example, at least about 50% or all uridines can be modified uridines, such as pseudouridine, N1-methyl-pseudouridine, and/or 5-methoxy-uridine. In some embodiments, substantially all uridines of the 5′ UTR and the 3′ UTR are replaced with pseudouridine or N1-methyl-pseudouridine. Other modified bases for use with the mRNA include 5-methyl cytidine and N6-methyl adenosine.

In some embodiments, the mRNA comprises a poly(A) tract. A poly(A) tract is a 3′ sequence that includes at least 20 nucleotides that are predominately A nucleotides. In exemplary embodiments, the polyA tract is at least about 20 nucleotides, at least about 50 nucleotides, or at least about 75 nucleotides, or at least about 100 nucleotides. In some embodiments, the polyA tract is about 100 nucleotides in length. In some embodiments the polyA tract may include non-A nucleotides or sequences of non-A nucleotides.

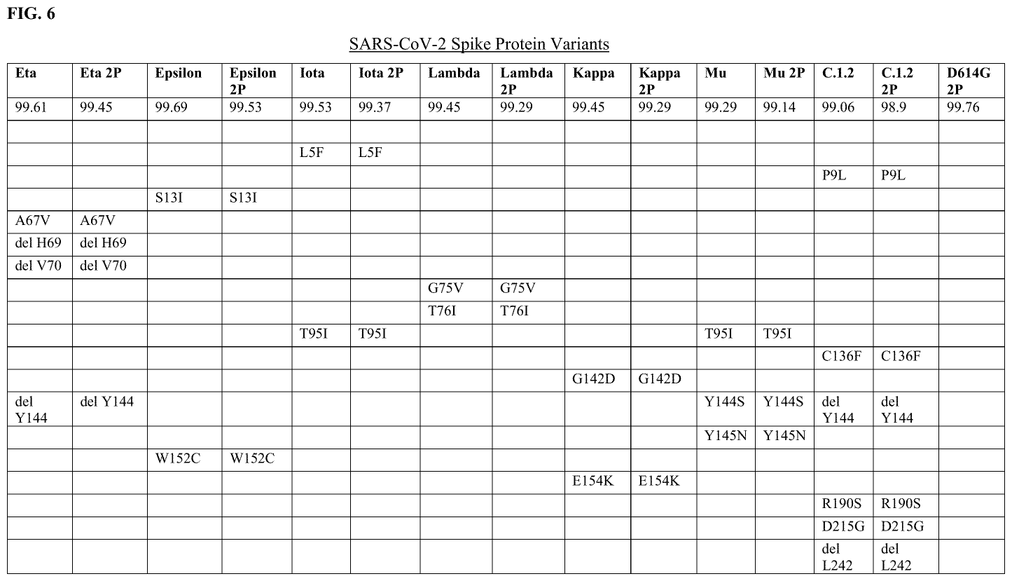

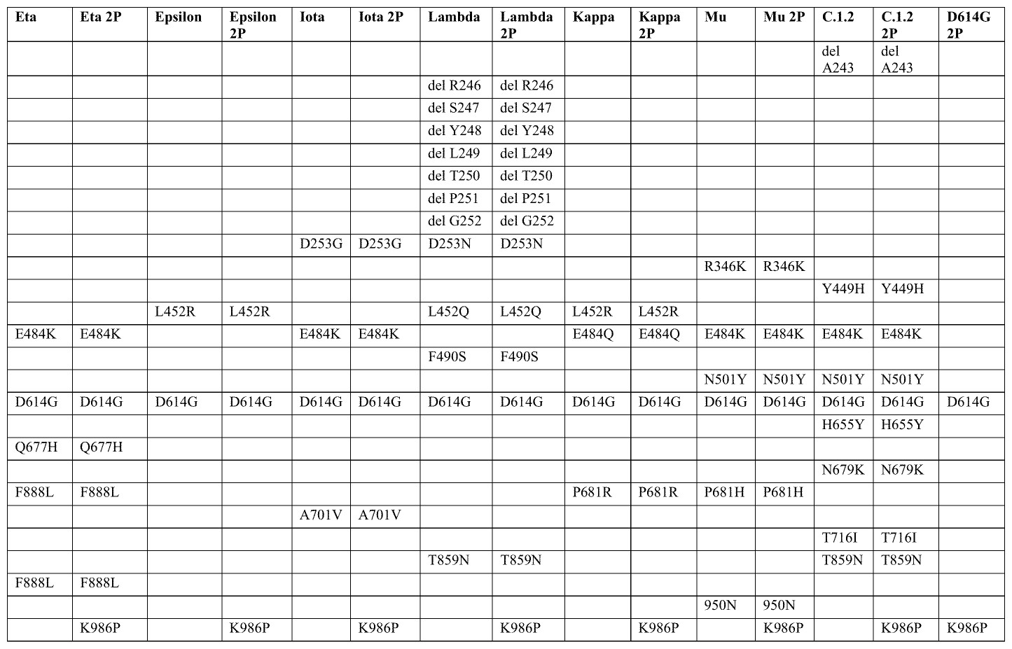

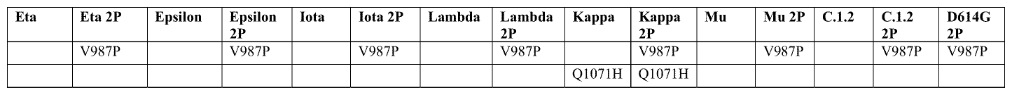

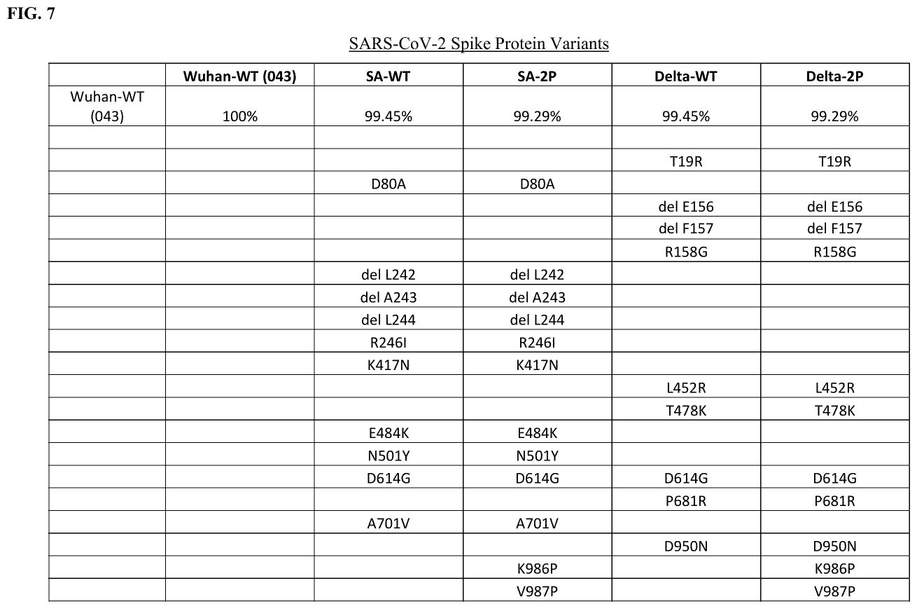

In various embodiments, the encoded spike protein has the amino acid sequence of SEQ ID NO: 5, optionally having one or more modifications. In embodiments, the modifications may be engineered or may be the same modifications found in naturally occurring mutant variants. In various embodiments, the spike protein is a wild type spike protein, that is, comprising the amino acid sequence of SEQ ID NO: 5 or a natural variant thereof. In some embodiments, the spike protein comprises from one to twenty, or from one to fifteen, or from one to ten, or from one to five amino acid substitutions. Exemplary amino acid substitutions can be selected from L5F, P9L, S13I, L18F, T19R, T20N, P26S, A67V, HV69-70del, G75V, T76I, D80A, T95I, C136F, D138Y, G142D, Y144del, Y144S, Y145N, W152C, EF156-157del, R158G, R190S, E154K, R190S, D215G, LA242-243del, LAL242-244del, R246I, RSYLTPG246-252del, D253N, D253G, R346K, K417N, K417T, Y449H, L452R, L452Q, T478K, E484K, E484Q, F490S, N501Y, A570D, D614G, H655Y, Q677H, N679K, P681H, P681R, A701V, T716I, T859N, F888L, D950N, S982A, K986P, V987P, Q1071H, T1027I, D1118H, and V1176F, Combinations include, for example, substitutions found in the spike protein of the “UK” or “alpha” variant of the SARS-COV-2 virus (HV69-70del, Y144del, N501Y, A570D, D614G, P681H, T716I, S982A, and D118H); substitutions found in the spike protein of the “South Africa” or “beta” variant of the SARS-COV-2 virus (D80A, LAL242-244del, R246I, K417N, E48K, N501Y, D614G, and A701V); substitutions found in the spike protein of the “Brazil” variant of the SARS-COV-2 virus (L18F, T20N, P26S, D138Y, R190S, K417T, E484K, N501Y, D614G, H655Y, T10271, and V1176F); and/or the substitutions of variants found in FIG. 6 and FIG. 7. In some embodiments, the encoded spike protein has the amino acid sequence of SEQ ID NO: 5. In such embodiments, the mRNA open reading frame may have a nucleotide sequence substantially corresponding to SEQ ID NO: 2 or 4. Optionally, where the in vitro transcription reaction includes nucleotides having modified bases, SEQ ID NO: 2 or 4 will include the modified nucleotides (e.g., modified uridine, such as pseudouridine or Nl-methyl-pseudouridine, replacing uridine). Other modified bases are described herein. In some embodiments, a plurality of mRNAs encoding different spike protein variants (e.g., one or more natural variants) can be contained in the same composition. In some embodiments, the wild type spike proteins or portions thereof are encoded on a single mRNA or different mRNA molecules.

In various embodiments, the encoded spike protein comprises a set of mutations listed in FIG. 6 or FIG. 7 for certain SARS-CoV-2 variants.

An exemplary mRNA encoding the SARS-CoV-2 spike protein is shown herein as SEQ ID NO: 2 or 4, which optionally contains one or more modified nucleotides as described herein. In some embodiments, the mRNA contains no modified nucleotides, or has all U nucleotides substituted with ψ or N1-methyl-pseudouridine.

In other aspects and embodiments, the mRNA encodes a therapeutic protein. In some embodiments, mRNA is targeted for expression in tissue or organs selected from liver (e.g., hepatocytes), skin (e.g., keratinocytes), skeletal muscle, endothelial cells, epithelial cells of various organs including the lungs, or hematopoietic or immune cells (e.g., T cells, B cells, or macrophages), for example. For example, the mRNA constructs may be designed to encode polypeptides of interest selected from vaccine targets, enzymes (including metabolic enzymes), antibodies or antigen-binding fragments thereof or antibody mimetics (including nanobodies or single chain antibodies), secreted proteins or peptides (including cytokines, growth factors, or soluble receptors for the same), plasma membrane proteins, cytoplasmic or cytoskeletal proteins, intracellular membrane bound proteins, nuclear proteins, proteins associated with human disease (including proteins having loss-of-function or gain-of-function mutations associated with human disease). In some embodiments, the therapeutic protein includes one or more cancer-associated epitopes (e.g., one or more mutations associated with cancer, including neoantigens), which may find use in a cancer vaccine. An exemplary embodiment in which the mRNA encodes for an antibody, open reading frames encoding heavy and light chains can be expressed from different mRNA molecules.

In some embodiments, the mRNA encodes one or more proteins of a virus or one or more polypeptides derived from virus proteins, for example, a DNA or RNA virus. Examples include those of the family Paramyxoviridae and/or genus Pneumovirinae or Morbillivirus. Example viruses include human metapneumovirus (hMPV), parainfluenza virus (hPIV), (types 1, 2, and 3), respiratory syncytial virus (RSV), and Measles virus (MeV). In some embodiments, the RNA virus is a coronavirus (CoV) (subfamily Coronavirinae, of the family Coronaviridae). In some embodiments, the coronavirus is a betacoronavirus, such as SARS-CoV or MERS-CoV. In some embodiments, the RNA virus is SARS-CoV-2, or a natural variant thereof. In other embodiments, the virus is a herpes virus, such as a herpes simplex virus or varicella zoster virus. In other embodiments, the virus is RSV, a hepatitis virus, or an adenovirus. In still other embodiments, the virus is an Ebola virus. In some embodiments the virus is an influenza virus. In other embodiments the virus is a Zika virus.

In some embodiments, the mRNA encodes one or more viral structural proteins or one or more polypeptides derived from virus proteins, such as a protein comprised in the viral envelop, such as a spike protein (S) for coronaviruses. Alternatively or in addition, the mRNA encodes other CoV structural proteins such as M (membrane) glycoprotein, E (envelope) protein, and/or N (nucleocapsid) protein. Alternatively, an mRNA encoding the spike protein or other structural protein can be encapsulated in particles that comprise or are decorated with one or more CoV structural proteins or portions thereof.

In some embodiments, the mRNA encodes one or more influenza virus proteins, such as neuraminidase (NA), hemagglutinin (HA), matrix protein 2 (M2), and/or nucleoprotein (NP).

In certain aspects, the disclosure provides a method for synthesizing the mRNA described herein. The method comprises contacting a linear DNA template encoding the mRNA under control of a promoter, with an RNA polymerase (e.g., T7 RNA polymerase) that recognizes said promoter and nucleotide triphosphate (NTP) reagents. In some embodiments, the process is performed according to an in vitro transcription process, as is known in the art. In still other embodiments, the process takes place as a cell-free process as described in WO 2020/205793, which is hereby incorporated by reference in its entirety.

In some embodiments, the DNA template, the RNA polymerase, and NTP reagents are contacted in a cell-free system synthesizing the NTP reagents from precursors. In some embodiments, the precursors comprise nucleosides, nucleotide monophosphate (NMP) reagents (e.g., which can be prepared by depolymerization of cellular RNA). For example, a cell-free method for synthesizing the mRNA may employ cellular RNA as a source of NTP substrates. The method may comprise incubating in a reaction mixture cellular RNA and one or more enzymes that depolymerize RNA under conditions wherein the cellular RNA is substantially depolymerized to produce 5′ nucleoside monophosphates. These RNA depolymerizing enzymes are eliminated or inactivated, and the nucleoside monophosphates are incubated with a second reaction mixture, which may comprise at least one polyphosphate (PPK) kinase and a phosphate donor. In some embodiments, the second reaction mixture comprises at least one cytidine monophosphate (CMP) kinase, at least one uridine monophosphate (UMP) kinase, at least one guanosine monophosphate (GMP) kinase, and at least one nucleoside-diphosphate (NDP) kinase, under conditions where nucleotide triphosphates (NTPs) are produced. The NTPs are contacted with at least one RNA polymerase (e.g., T7 RNA polymerase) and the DNA template encoding the mRNA. In some embodiments, the reaction may further comprise reagents for capping the mRNA produced (i.e., by co-transcriptional capping), or optionally, this step is performed subsequently (i.e., by enzymatic means). Further, in some embodiments, the polyA tail is not encoded in the DNA template, and is added post-transcriptionally (e.g., in a separate reaction) by a polyA polymerase in the presence of ATP. In some embodiments, the polyA tract is encoded by the DNA template.

Other cell-free processes for mRNA synthesis are described in U.S. Pat. No. 10,858,385 and WO 2020/205793, which are hereby incorporated by reference in their entireties.

In some embodiments, the present disclosure provides an mRNA composition comprising the mRNA as described herein, or synthesized according to the method described herein, and combined with a transfection agent or encapsulated within a delivery vehicle.

Any of the known transfection agents and delivery vehicles may be used, including those that sequestrate the mRNA into vesicles. An exemplary transfection agent is lipofectamine. Alternatively, the delivery vehicle comprises a lipid nanoparticle (LNP), having the mRNA encapsulated therein. In various embodiments, the LNPs comprise a cationic or ionizable lipid, a neutral lipid or phospholipid, a structural lipid such as a cholesterol or cholesterol moiety, and a PEGylated lipid. Lipid particle formulations that find use with embodiments of the present disclosure include those described in U.S. Pat. Nos. 9,738,593; 10,221,127; and 10,166,298, which are hereby incorporated by reference in their entirety. See also, Schoenmaker, L., Witzigmann, D., Kulkarni, J. A., Verbeke, R., Kersten, G., Jiskoot, W., & Crommelin, D. J. (2021), mRNA-lipid nanoparticle COVID-19 vaccines: Structure and stability. Int. J. Pharm., 601, 120586.

In some embodiments, the lipid nanoparticle (or LNP) comprises a structural lipid. Exemplary structural lipids can be selected from one or more of cholesterol, fecosterol, sitosterol, ergosterol, campesterol, stigmasterol, brassicasterol, tomatidine, ursolic acid, and tocopherols (e.g., alpha tocopherol). In some embodiments, the structural lipid is cholesterol.

In some embodiments, the LNP comprises one or more phospholipids. Exemplary phospholipids are selected from the group consisting of cardiolipins, sterol modified lipids (modified with a cholesterol moiety attached at the sn-2 carbon of the glycerol backbone), mixed-acyl glycerophospholipids, and symmetrical acyl glycerophospholipids. Head groups for acyl glycerophospholipids include, for example, phosphatidic acid, lysophosphatidic acid, phosphatidylcholine, phosphatidylethanolamine, phosphatidylglycerol, phosphoinositides, and phosphatidylserine. Exemplary phospholipids are selected from 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-pho spho cho line (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanol amine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethano lamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethano lamine, 1,2-dioleoyl-sn-glycero-3-phospho-rac-(1-glycerol) sodium salt (DOPG), and sphingomyelin.

In some embodiments, the lipid nanoparticle composition further comprises one or more PEG lipids. A PEG lipid is a lipid modified with polyethylene glycol. Exemplary PEG lipids are selected from one or more of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol. A PEG lipid may be selected from PEG-c-DOMG, PEG-DMG, PEG-DLPE, PEG-DMPE, PEG-DPPC, PEG-Cholesterol, PEG tocopherol, or a PEG-DSPE lipid.

In some embodiments, the lipid nanoparticle composition comprises 1,2-dimyristoyl-rac-glycero-3-methoxypolyethylene glycol-2000 (DMG-PEG).

In some embodiments, the lipid nanoparticle composition comprises a structural lipid, a PEG lipid, and a phospholipid, each optionally according to the preceding paragraphs. In exemplary embodiments, the LNP comprises 1,2-distearoyl-sn-glycero-3-phosphocholine (DSPC), cholesterol, and 1,2-dimyristoyl-rac-glycero-3-methoxypolyethylene glycol-2000 (DMG-PEG).

In some embodiments, the formulated mRNA is delivered in vivo (e.g., by injection or orally). In still other embodiments, the mRNA described herein is introduced into a cell ex vivo, and the cell may be administered to a patient. Exemplary cells include non-adherent cells such as white blood cells (T cells including CAR-T cells, B cells, dendritic cells, or macrophages), stem cells, or fibroblasts.

In some aspects, the disclosure provides a method for preventing or reducing the probability of SARS-CoV-2 infection in a patient. In these embodiments, the method comprises administering the mRNA vaccine expressing SARS-CoV-2 spike protein and/or other SARS-CoV-2 structural protein as described herein. In some embodiments, the mRNA vaccine is administered as a single dose. In some embodiments, the mRNA vaccine is administered as multiple (e.g., two) doses, with a booster one, two, or three weeks after the initial dose.

In some aspects, the disclosure provides a method for expressing a therapeutic protein in a patient, comprising administering the mRNA composition described herein. For example, diseases, disorders, and/or conditions for treatment or prevention, include: autoimmune disorders (e.g., diabetes, lupus, multiple sclerosis, psoriasis, rheumatoid arthritis); inflammatory disorders (e.g., arthritis, pelvic inflammatory disease); infectious diseases (e.g., viral infections, bacterial infections, fungal infections, and sepsis); neurological disorders (e.g., Alzheimer's disease, Huntington's disease; autism; Duchenne muscular dystrophy); cardiovascular disorders (e.g., atherosclerosis, hypercholesterolemia, thrombosis, clotting disorders, angiogenic disorders such as macular degeneration); metabolic disorders and liver disorders (e.g., ornithine transcarbamylase deficiency); proliferative disorders (e.g., cancer, benign neoplasms); respiratory disorders (e.g., chronic obstructive pulmonary disease or idiopathic pulmonary fibrosis); digestive disorders (e.g., inflammatory bowel disease, ulcers); musculoskeletal disorders (e.g., fibromyalgia, arthritis); endocrine, metabolic, and nutritional disorders (e.g., diabetes, osteoporosis); urological disorders (e.g., renal disease); psychological disorders (e.g., depression, schizophrenia); skin disorders (e.g., wounds, eczema); and blood and lymphatic disorders (e.g., anemia, hemophilia).

Exemplary diseases characterized by dysfunctional or aberrant protein activity include cystic fibrosis, sickle cell anemia, epidermolysis bullosa, amyotrophic lateral sclerosis, and glucose-6-phosphate dehydrogenase deficiency. In various embodiments, the present disclosure provides a method for treating such conditions or diseases in a patient by introducing an mRNA encoding for a protein that overcomes the aberrant protein activity present in the cell of the patient. Specific examples of a dysfunctional protein are the missense mutation variants of the cystic fibrosis transmembrane conductance regulator (CFTR) gene, which produce a dysfunctional protein variant of CFTR protein, which causes cystic fibrosis.

Other diseases characterized by missing or substantially diminished protein activity (such that proper, normal or physiological protein function does not occur) include cystic fibrosis, Niemann-Pick type C, β thalassemia major, Duchenne muscular dystrophy, Hurler

Syndrome, Hunter Syndrome, and Hemophilia A. Such proteins may not be present or are essentially non-functional. The present disclosure provides a method for treating such conditions or diseases in a patient by introducing mRNA provided herein, wherein the mRNA encodes for a protein that replaces the protein activity missing from the target cells of the patient.

As used herein, the term “about” means ±10% of an associated numerical value.

As used herein, the word “include,” and its variants, is intended to be non-limiting, such that recitation of items in a list is not to the exclusion of other like items that may also be useful in the compositions and methods of this technology.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Other aspects and embodiments of the invention will be apparent from the following Examples.

### EXAMPLES

The SARS-CoV-2 coronavirus was first described in Wuhan, China in December 2019, and has resulted in over 100 million cases of associated disease (COVID-19) and over two million deaths globally. In the US, and as of February 2021, over 25 million COVID-19 cases have been identified and over 450,000 deaths have occurred due to this pandemic. Development of preventive vaccines is critical for the control of SARS-CoV-2 infection in the population.

### Example 1

### Design of Synthetic mRNA Encoding SARS-CoV-2 Spike Glycoprotein

This example describes design of synthetic messenger RNA (mRNA) molecules encoding the full-length spike (S) glycoprotein of the coronavirus SARS-CoV-2, which may be encapsulated in lipid nanoparticles (LNPs) for use as vaccines against SARS-CoV-2. The mRNA molecules have the following characteristics:

(1) Encode a full-length wild-type spike protein, i.e., without prefusion stabilization or other changes that alter the protein's conformation as an antigen;

(2) 5′ and 3′ untranslated regions (UTRs) derived from those of the human beta-globin (HBG) gene;

(3) The initial transcribed sequence (ITS) present at the 5′ end of the 5′ UTR (SEQ ID NO: 7) is an artificial sequence that improves titer and quality in RNA synthesis reactions; and

(4) A unique codon-optimized open reading frame.

According to the following example, two mRNA vaccines against COVID-19 were produced. These vaccines involve one mRNA product encapsulated in a solid lipid nanoparticle for delivery. With the exception of the 5′ cap, a first design consists of unmodified A, C, G, and U nucleotides (referred to herein as GLB-COV-2-042). In a second design, the uridine nucleotides are replaced by pseudouridine nucleotides (referred to herein as GLB-COV-2-043).

While 042 and 043 both code for the wildtype Wuhan spike protein, in other embodiments, mRNA vaccines against COVID-19 may exchange this portion of the sequence for a sequence that codes for any variant version of the spike protein, including those set forth in FIG. 6, or for a sequence that codes for a different antigen. Similarly, mRNA vaccines of the present disclosure may code for antigenic proteins for any given virus.

Molecule Design

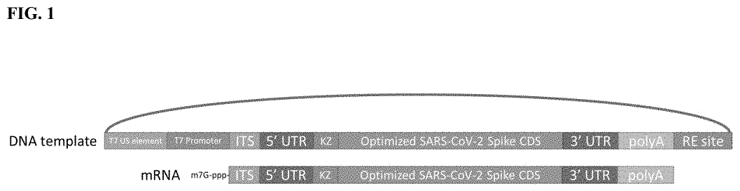

FIG. 1 illustrates a DNA construct for in vitro or cell-free synthesis of mRNA encoding the SARS-CoV-2 spike protein. mRNAs are synthesized using unmodified nucleotides (GLB-COV-2-042), or optionally with uridine nucleotides replaced by pseudouridine (GLB-COV-2-043). The plasmid design includes a T7 promoter, a 5′ UTR which includes an ITS and Kozak sequence, codon-optimized spike protein gene, 3′ UTR, Poly(A) sequence, and a restriction endonuclease recognition site for linearization of the template. In other designs of the present disclosure, the codon-optimized spike protein gene may be replaced with a wild type or codon-optimized gene for any protein that is desired to be expressed, such as an antigen, an antibody, or any therapeutic protein.

The mRNA molecules have the nucleotide sequence of SEQ ID NO: 6 and 7, which includes the following elements (shown emphasized in SEQ ID NO: 6 and 7): A 5′ cap 1 structure ^{m7}G(5′)ppp(5′)(^{2′OMe}A), as well as an initial transcribed sequence (ITS) of 15 nucleotides (SEQ ID NO: 11). The ITS improves production titers, reduces sequence-to-sequence variability in titer and improves quality by reducing abortive transcription products. The first nucleotide of the ITS is A, which also allows for efficient co-transcriptional capping using CleanCap ™ AG. Following the ITS, the 5′ untranslated region (UTR) further comprises a sequence derived from the human (3-globin gene (NG 059281.1) (SEQ ID NO: 12). The 5′ UTR further includes a strong Kozak sequence before the initiation codon (SEQ ID NO: 13). The open reading frame (ORF) encoding the amino acid sequence of the wild-type spike (S) glycoprotein from SARS-CoV-2 (QHD43416.1) is represented by SEQ ID NO: 5. The nucleotide sequence of the ORF is codon-optimized to maximize GC-content and minimize U-content to reduce off-target immunogenicity, and to ablate Esp3I (SEQ ID NO: 1) or BspQI (SEQ ID NO: 3) and BsmB1 (SEQ ID NO: 1 and SEQ ID NO: 3) recognition sites in the open reading frame. The 3′ untranslated region from the human β-globin gene follows the stop codon and is represented by SEQ ID NO: 14. The mRNA has a 3′ poly(A) sequence of 100 nucleotides (SEQ ID NO: 15). The same construct may be used for a different vaccine or for a therapeutic product by replacing the ORF encoding the amino acid sequence of the SARS-CoV-2 spike protein, with an ORF encoding the amino acid sequence of any known antigen or therapeutic protein.

Molecule Production and Biochemical Characterization

mRNAs were produced using the cell-free production platform as described in WO 2020/205793, which is hereby incorporated by reference in its entirety. Briefly, nucleoside 5′-monophosphates and cap analog (C1eanCap™ AG reagent, TriLink BioTechnologies, Inc.) were incubated in the presence of NMP kinases, NDP kinase, polyphosphate kinase, RNA polymerase, and a linearized DNA template to produce the mRNA molecules. Linearized DNA templates were derived from a minimal pUC19-derived plasmid, which encoded the amino acid sequence of SEQ ID NO: 5, with a 3′ Esp3I site for linearization.

Plasmids were propagated in E. coli strain DH10b (Thermo Fisher), purified via Plasmid Giga Kit (Qiagen), linearized by treatment with restriction enzyme (e.g., Esp3I, New England BioLabs), and the linearized plasmid was purified by phenol-chloroform extraction. After RNA synthesis, the plasmid template was removed by treatment with TURBO DNase (Thermo Fisher), and the RNA was recovered by lithium chloride precipitation. mRNAs were further purified by reverse phase-ion pair HPLC, concentrated to 1 mg/mL, then frozen.

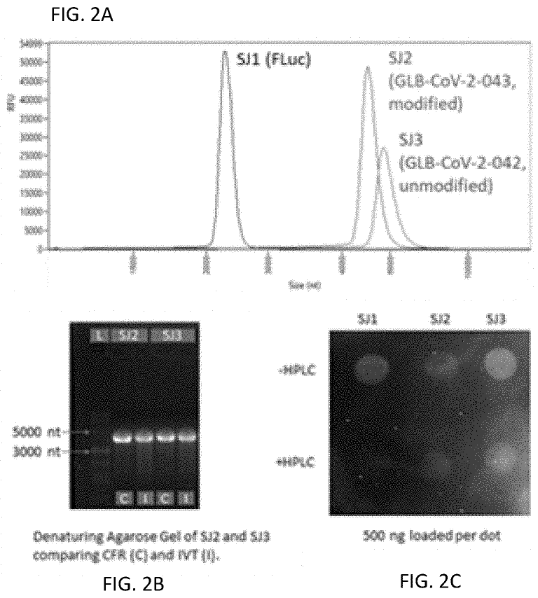

RNAs were characterized for purity and quality in biochemical assays. Capillary electrophoresis (FIG. 2A) showed homogenous populations of RNA in the expected size range with the unmodified construct (GLB-COV-2-042) sizing larger than the modified construct having pseudouridines (GLB-COV-2-043). To confirm the relative size of each construct, denaturing agarose gel electrophoresis using formaldehyde was performed (FIG. 2B) and compared to controls produced by conventional in vitro transcription (IVT). As expected, both molecules sized similarly by fully-denaturing gel electrophoresis regardless of production method or the presence of modifications. RNA products were also analyzed for residual double-stranded RNA (dsRNA) by immunoblot using the J2 antibody. Here, HPLC-purified RNAs generally exhibited lower dsRNA content than pre-purification samples (FIG. 2C).

In Vitro Characterization

Spike protein expression was analyzed by western blot using HEK293T cells (ATCC) as shown in FIG. 3B. Cells were seeded at 3×10⁵ cells/well of 24-well plates in Opti-MEM (Thermo Fisher), 10% fetal calf serum, 1× penicillin/streptomycin/amphotericin B. Cells were incubated overnight, resulting in ˜80% confluence. Cells were then transfected with two different mRNA quantities (500 ng or 1500 ng) using Lipofectamine MessengerMAX™ reagent (Thermo Fisher) following the manufacturer's recommendations. At 48 hours post-transfection (hpt), supernatants were removed, monolayers were disrupted with 100 μL passive lysis buffer (Promega). Insoluble debris was pelleted by centrifugation, and 15 μL was resolved by SDS-PAGE under reducing conditions. Proteins were blotted to nitrocellulose, blocked (Odyssey Blocking Buffer), and probed with a rabbit primary antibody that binds to SARS-CoV-2 spike receptor binding domain (RBD) (1:2000, SINO Biological) followed by incubation with a secondary antibody (goat anti-rabbit HRP 1:15000, Jackson ImmunoResearch Laboratories, Inc.). Recombinant his-tagged SARS-CoV-2 spike S1 domain (SINO Biological) was loaded as a positive control. FIG. 3B shows that the spike protein can be detected at 48 hpt, meaning that the transfection of both mRNAs, the unmodified GLB-COV-2-042 (SJ3) and the modified GLB-COV-2-043 (SJ2), are actively translated and properly express the spike protein. The full-length spike protein is detected at a molecular weight of approximately 180 kDa and there are some variations given glycosylation.

Spike protein expression was further analyzed by enzyme-linked immunosorbent assay (ELISA) as shown in FIG. 3C and FIG. 3D. 293T cells were seeded at 2×10⁴ cells/well in 96-well plates for overnight incubation, resulting in ˜80 to 90% confluence. Transfection reagents and conditions employed the Lipofectamine MessengerMAX™ reagent per manufacturer's recommendations, but with 100 ng per well of RNA generated by standard in vitro transcription reactions (IVT-RNA), or with cell free transcription reaction essentially as described in WO 2020/205793 (GLB RNA). After overnight incubation, cell monolayers were used as targets in an ELISA. The positive control for this assay is shown in FIG. 3C, using different quantities of soluble spike recombinant protein (SINO Biologicals) as the coating agent. FIG. 3D shows results from mRNA transfected cells. Cells were prepared for the ELISA by fixation with 100 μL acetone/PBS (80/20) for 1 min. Wells were air-dried, rehydrated with 200 μL PBS, and then blocked with 1% BSA in PBS for 30 min at 37° C. To perform ELISAs, monoclonal anti-spike antibody (SINO Biologicals) was added for 30 min at 37° C., followed by washes and the addition of goat anti-human IgG conjugated to horse radish peroxidase (Southern Biologicals) for 30 min at 37° C. Plates were developed with TMB substrate (KPL) and reactions were stopped with 1M phosphoric acid. Readings were at OD 450 nm. Three negative controls were included in the experiment: mock-transfected cells, a firefly luciferase mRNA, and an EGFP mRNA. These transfections identified assay backgrounds and showed that mRNA vaccine samples scored significantly above background, indicating positive spike protein expression.

As shown in FIG. 3D, the two mRNA molecules: CoV-2-042 (unmodified) and CoV-2-043 (pseudouridine-modified), whether they are generated by standard IVT or GLB RNA synthesis, showed expression of the SARS-CoV-2 full-length spike protein with very similar potency.

mRNA was encapsulated for animal studies using an LNP substantially as described in Schoenmaker, L., Witzigmann, D., Kulkarni, J. A., Verbeke, R., Kersten, G., Jiskoot, W., & Crommelin, D. J. (2021), mRNA-lipid nanoparticle COVID-19 vaccines: Structure and stability. Int. J. Pharm., 601, 120586.

### Example 2

### Constructs with ITS Comprising A-Start Produce Capped RNA at High Titer and Purity Using Co-Transcriptional Capping Reagents

A pair of mRNA molecules were designed and produced in cell-free reactions. Both molecules consisted of a similar sequence architecture, incorporating a 5′ ITS GGGAGACCAGGAATT (SEQ ID NO: 10) or AGGAGACCAGGAATT (SEQ ID NO: 11). mRNA sequences, were encoded on a pUC-19 derived plasmid template along with a T7 promoter at the 5′ end and a restriction endonuclease recognition site. Plasmids were propagated in E. coli strain DH10b, purified by Plasmid Giga Kits (Qiagen), linearized by digestion with Esp3I restriction endonucleases (New England BioLabs), and further purified by phenol-chloroform extraction. RNA synthesis reactions were performed using a cell-free production platform as described in WO 2020/205793, which is hereby incorporated by reference in its entirety. Reactions producing capped RNAs also included C1eanCap™ AG reagent (TriLink BioTechnologies). Template DNA was removed by treatment with DNase I, then RNA was recovered by lithium chloride precipitation.

Recovered RNA was quantified by UV absorbance at 260 nm and analyzed for size and quality using a 2100 Bioanalyzer instrument (Agilent Technologies).

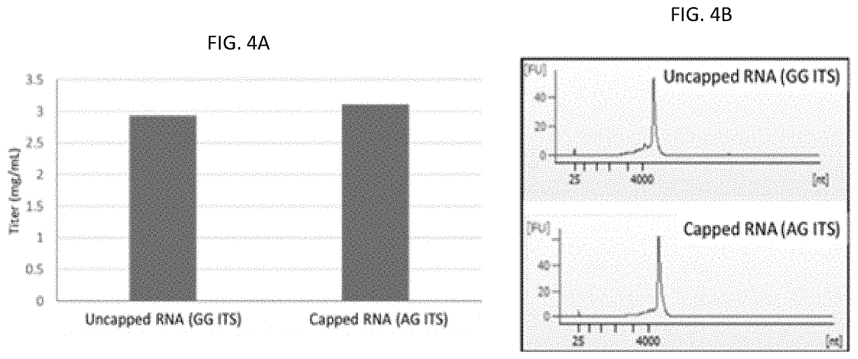

As shown in FIG. 4A and FIG. 4B, cell-free reactions producing capped RNA using CleanCap™ AG and an AG ITS (SEQ ID NO: 11) achieved similar titers as reactions producing uncapped RNA using a GG ITS (SEQ ID NO: 10) (FIG. 4A). Analysis by Bioanalyzer demonstrated RNA products from both reactions were of the expected size and similar purity (FIG. 4B).

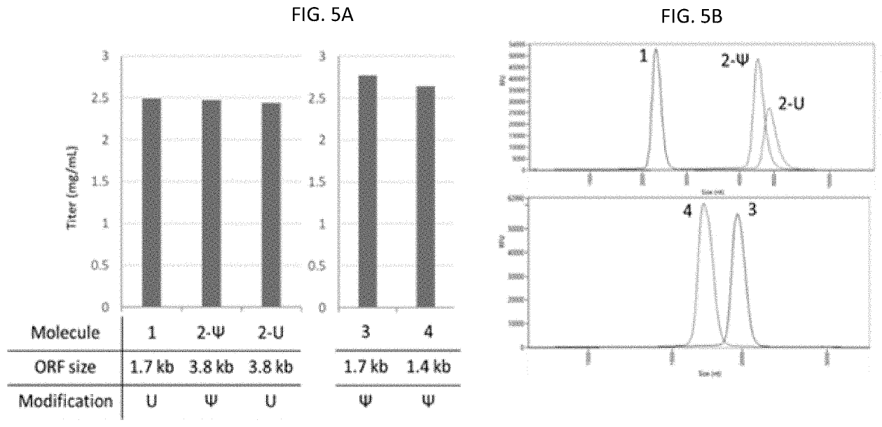

ITS in mRNAs encoding different proteins resulted in consistent production titers and molecule quality. As shown in FIG. 5A and FIG. 5B, cell-free reactions producing capped RNA using CleanCap™ AG and the ITS of SEQ: ID NO: 11 produced consistent titers across multiple open reading frame sequences (FIG. 5A). RNA products of these reactions migrated at the expected sizes. All molecules were produced with similarly high purity (FIG. 5B).

A family of mRNA molecules were designed and produced in cell-free reactions. Sequences incorporated the 5′ ITS AGGAGACCAGGAAUU (SEQ ID NO: 11), a 5′ UTR sequence from the human beta-globin (HBG) gene (SEQ ID NO: 12), an open reading frame, a 3′ HBG UTR (SEQ ID NO: 14), and a 100-nucleotide polyA tail (SEQ ID NO: 15). RNA synthesis reactions were performed using a cell-free production platform as described in WO 2020/205793, which is hereby incorporated by reference in its entirety. Reactions producing capped RNAs also included CleanCap™ AG reagent (TriLink BioTechnologies). Plasmids were propagated in E. coli strain DH10b, purified by Plasmid Giga Kits (Qiagen), linearized by digestion with Esp3I or BspQI restriction endonucleases (New England BioLabs), and further purified by phenol-chloroform extraction. Template DNA was removed by treatment with DNase I, then RNA was recovered by lithium chloride precipitation. Recovered RNA was quantified by UV absorbance at 260 nm and analyzed for size and quality using a Fragment Analyzer instrument (Agilent Technologies).

### Example 3

### Neutralizing Serum Antibody Titers

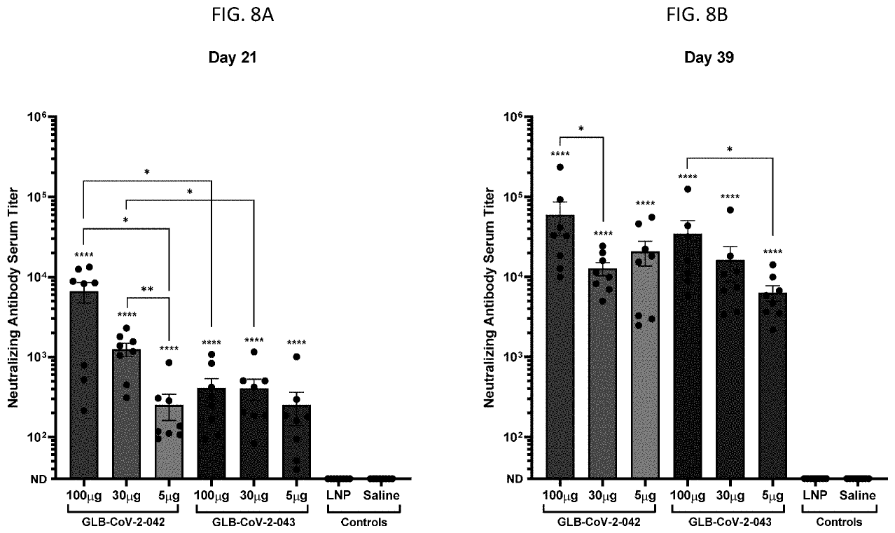

Immunogenicity and protection from SARS-CoV-2 for GLB-COV2-042 and -043 were evaluated in a golden Syrian hamster model. The hamsters were vaccinated with 100 μg, 30 μg, or 5 μg of GLB-COV2-042 or GLB-COV2-043 at the outset of the study and again on day 21. Serum Nab titers against the WA-1 strain of SARS-CoV-2 were quantified by FRNT. Results on days 21 and 39 are shown in FIG. 8A and FIG. 8B, including means and standard deviations. Data from the controls were combined for statistical analyses. Statistical analyses were performed using rank-based Mann-Whitney and Holm-Šid\acute{α}k multiple comparisons tests. Statistical signifiers above the bars represent significance between that group and the controls. *=p<0.05; ***=p<0.005; ****=p<0.001.

Immunization at all doses for each variant, measured at day 21 and day 39, significantly (p<0.001) increased the serum neutralization geometric mean titer compared to mock-vaccinated or control animals receiving LNP or saline alone. These data demonstrated that both GLB-COV2 mRNA vaccines induced high titers of neutralizing anti-SARS-CoV-2 antibodies.

### Example 4

### Morbidity in Vaccinated Hamsters Following SARS-CoV-2 Challenge

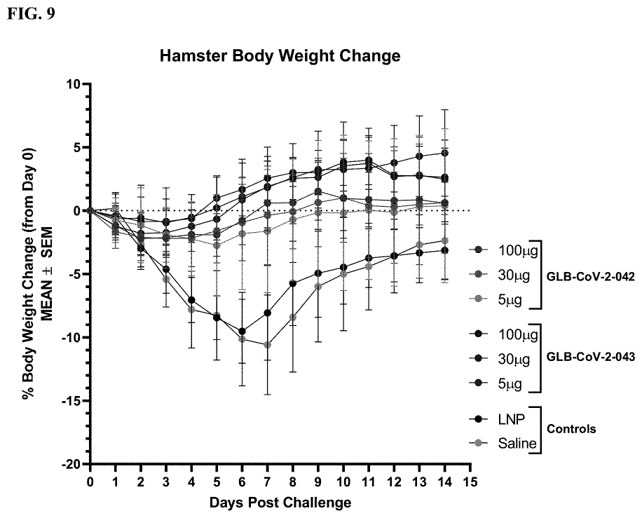

To evaluate morbidity, hamsters challenged by SARS-CoV-2 were vaccinated with 100, 30, or 5 μg of GLB-COV2-042 or GLB-COV2-043 on days 0 and 21 and intranasally challenged on day 42 with the Wuhan strain of SARS-CoV-2. Percent body weight change of each vaccination group was measured over 14 days post-infection. Results are shown in FIG. 9. As shown in FIG. 9, mock vaccinated and control hamsters lost up 10% of initial bodyweight on average by days 7 post challenge before beginning to regain weight. By contrast, two doses of both vaccines protected the hamsters from severe weight loss. Hamsters vaccinated with 043 variant gained weight more rapidly than those vaccinated with 042 variant. Nevertheless, all the subjects recovered baseline bodyweight by 9 days post challenge.

### Example 5

### Infectious Virus Titers and Viral RNA Levels in Lungs and Nasopharynx

In order to evaluate the effects of vaccination on virus titers and viral RNA levels in the lungs and nasopharynx, eight hamsters were vaccinated with 5, 30, or 100 μg of GLB-COV2-042 or GLB-COV2-043 on days 0 and 21 and challenged on day 42 with the Wuhan strain of SARS-CoV-2. At days 2 and 4 post challenge, virus titers and RNA levels were evaluated in lung and nasopharynx tissue. To quantify viral RNA levels, RT-qPCR was used to detect the number of nucleocapsid (N) gene copies per gram of tissue. Infectious virus titers were determined by TCID₅₀ assay and reported as TCID₅₀/gram of tissue.

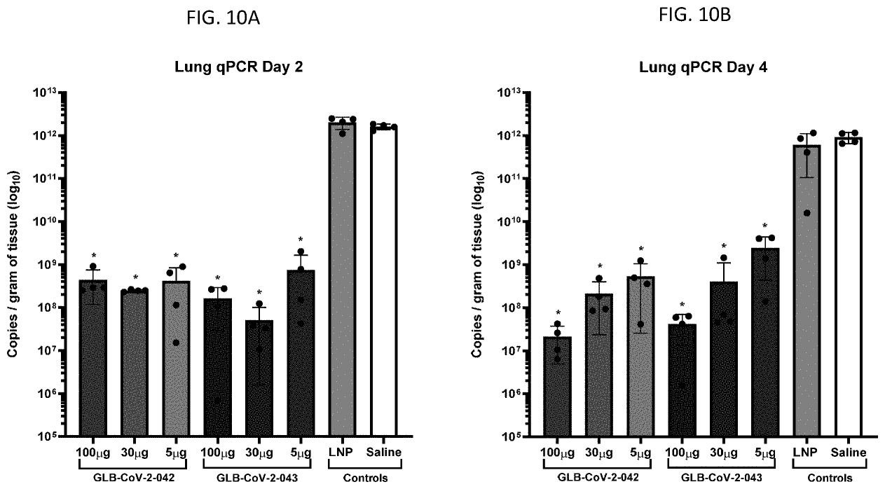

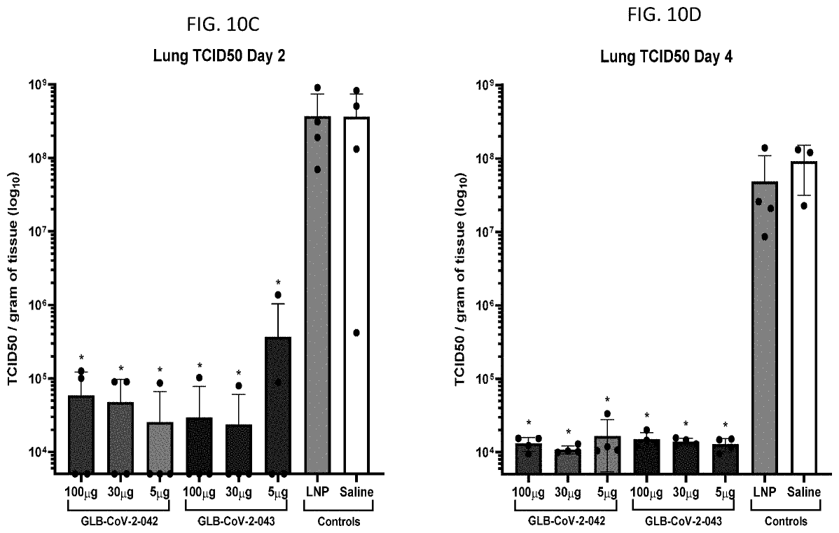

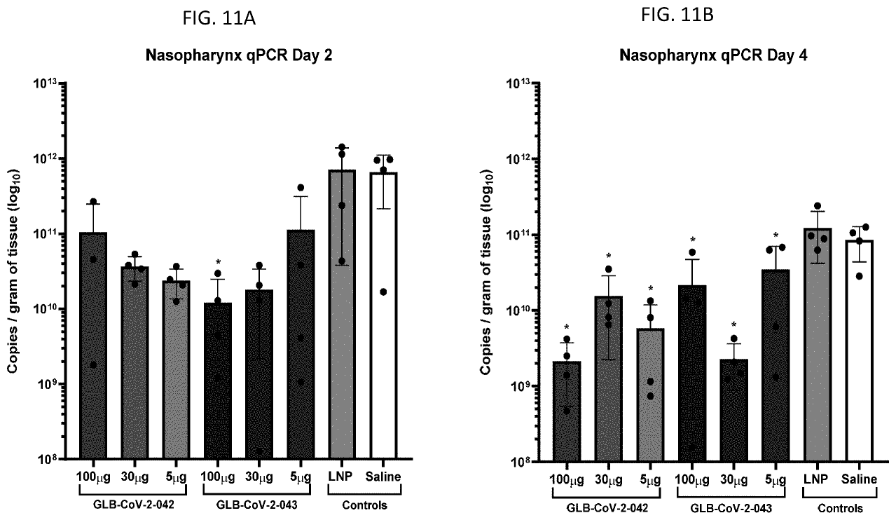

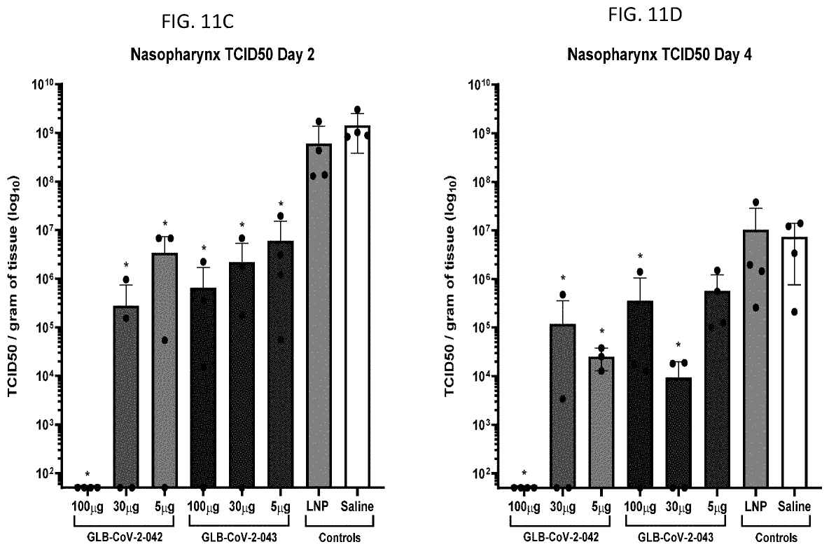

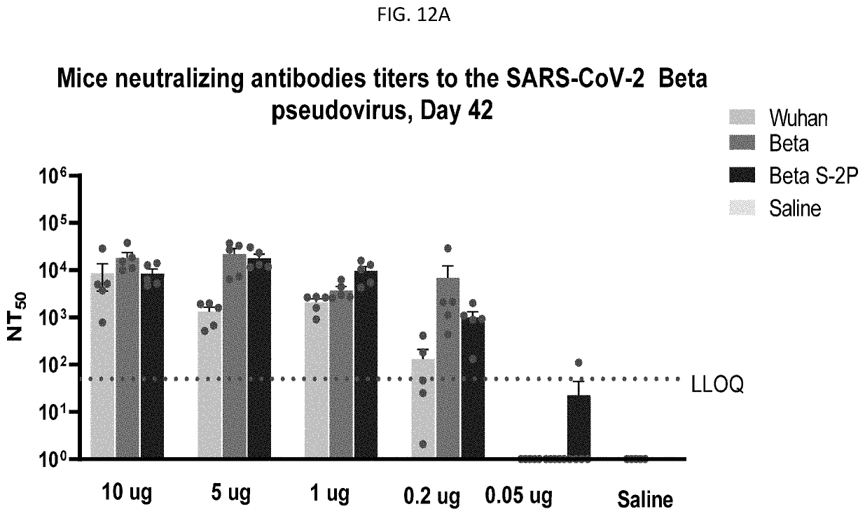

Results for lung tissue are shown in FIGS. 10A-D and nasopharynx results are shown in FIGS. 11A-D. Means and standard deviations are shown. Data from controls were combined for statistical analysis. Statistical analyses were performed using rank-based Mann-Whitney and Holm-Šid\acute{α}k multiple comparisons tests. Statistical signifiers above the bars represent significance between that group and the controls. *=p<0.05; ***=p<0.005; ****=p<0.001.

FIG. 10A and FIG. 10B show number of copies of N gene copies in lung tissue at days 2 and 4 post infection, respectively. FIG. 10C and FIG. 10D show TCID₅₀ values at days 2 and 4 post infection, respectively. FIG. 10A demonstrates that immunization with two doses of all concentrations for both variants reduced the number of N gene copies more than 1,000-fold at 2 dpi without any significant difference between the doses. Similar results were found for 4 dpi as disclosed in FIG. 10B.

FIG. 10C and FIG. 10D show TCID₅₀ values as measured in lung tissue at two and four days post challenge. Viral replication as evidenced by the TCID₅₀ values for both 042 and 043 were 1000-fold lower than controls at 2 dpi as set forth in FIG. 10C. By day 4 post infection, virus titers in vaccinated test animals had declined to the limit or near the limit of detection, with lung viral titers of controls remaining at 1 ×10⁸, as shown in FIG. 10D.

Nasopharynx results from the same assays are shown in FIG. 11. These figures demonstrate the effectiveness of reducing SARS-CoV-2 in the nasopharynx while no significant difference in copies of N was detected for vaccinated animals vs. control at 2 dpi (FIG. 11A). By day 4, the differences between vaccine and control groups reached significance, but RNA levels remained high in all groups (FIG. 11B). Significant reductions in infectious titers in nasal washes were seen in immunized animals as compared to control groups at both days 2 and 4 post challenge (FIGS. 11C-D, respectively).

### Example 6

### Mice Neutralizing Antibodies Titers to SARS-CoV-2 and Variants

To measure the neutralizing antibody levels in sera samples isolated from mice immunized with GLB mRNA vaccine candidates, a pseudovirus neutralization assay was used to measure functional neutralizing antibody responses against the Wuhan (wildtype) SARS-CoV-2 and variants of concern (VoC) Beta and Delta. The Pseudovirus is a nonreplicating and viral particle that uses the murine leukemic virus (MLV) backbone and expresses the spike protein from SARS-CoV-2. The virus has a luciferase reporter plasmid. Wild type C57BL/6 mice were immunized with the vaccine candidates. The mice sera were incubated with pseudotyped virus for 1 hour and added to a HEKACE2 (cell expressing the human ACE2 receptor) cell line to measure the relative light units (RLU) given by the luciferase luminescence when the pseudoviral particles enter the cells. If the serum is highly neutralizing, the luciferase expression will be lower as the pseudovirus will be blocked from infecting the HEKACE2 cells. The final 50% neutralizing titers (NT50) are calculated based on a non-linear regression curve fit using GraphPad Prism 9 software.

Mice were immunized with the vaccine candidates GLB-COV-2-043 (Wuhan), GLB-COV-2-047 (Beta), and GLB-COV-2-048 (Beta S-2P) on a regimen of prime and boost 21 days apart (prime day 0 and boost day 21). The mice were euthanized on day 42, and blood (serum) and lymphoid tissue (spleen) were collected to analyze humoral and cellular immune responses.

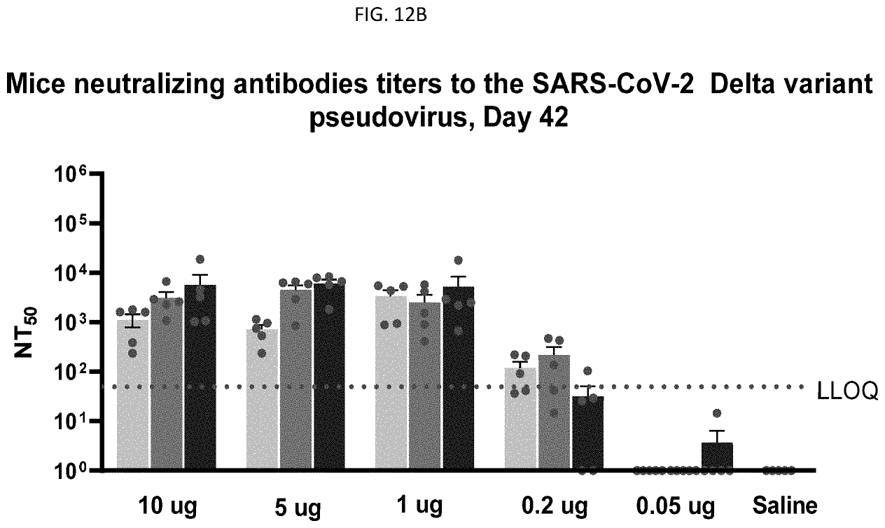

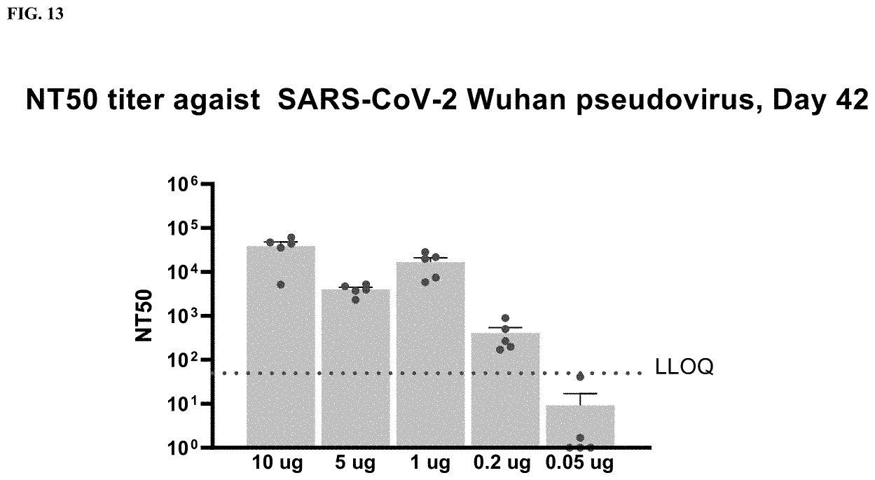

FIG. 12A and FIG. 12B show results for GLB-COV-2-043 (Wuhan) GLB-COV-2-047 (Beta), and GLB-COV-2-048 (Beta, prefusion stabilized) as against the Beta variant of SARS-CoV-2 (FIG. 12A) and the Delta variant of SARS-CoV-2 (FIG. 12B). FIG. 13 shows results for GLB-COV-2-042 against Wuhan SARS-CoV-2. As shown in FIG. 12A, FIG. 12B, and FIG. 13, all three vaccine candidates induced strong homologous and heterologous neutralizing responses in mice at day 42. The nAb titers were higher than the limit of quantification (LLOQ) and the Saline group at higher doses of the mRNA vaccines 10 μg, 5 μg and 0.2 μg. The statistical analysis was performed by non-parametric Kruskal-Wallis test, followed by the Dunn's multiple comparisons test and adjustment of the p-value. p<0.05 was considered statistically different.

### Example 7

### Cellular Responses to GLB Vaccine Candidates in Mice by T Cell Assay

Mice were immunized with the vaccine candidates GLB-COV-2-043 (Wuhan), GLB-COV-2-047 (Beta), and GLB-COV-2-048 (Beta, prefusion stabilized) on a regimen of prime and boost 21 days apart (prime day 0 and boost day 21). The mice were euthanized on day 42. The lymphoid tissues (spleen) were collected and analyzed for T CD4+and T CD8+immune responses.

From the harvested spleens, splenocytes were isolated from the mice immunized with 10 μg or 1 μg of GLB-COV2-043,-047, and -048's vaccine candidates and in vitro stimulated with Wuhan, β, and κ SARS-CoV-2 spike protein-peptide pools, overnight. The next day, the cells were analyzed by multicolor flow cytometry for intracellular cytokine production.

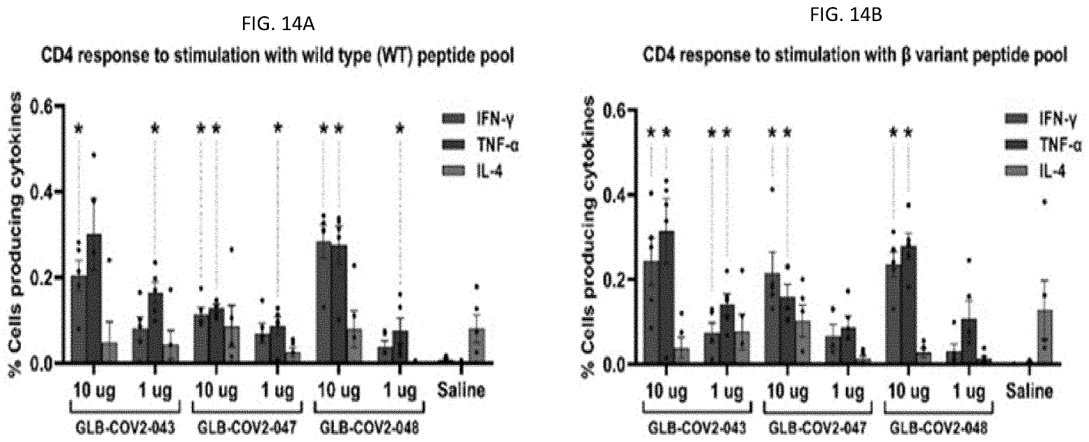

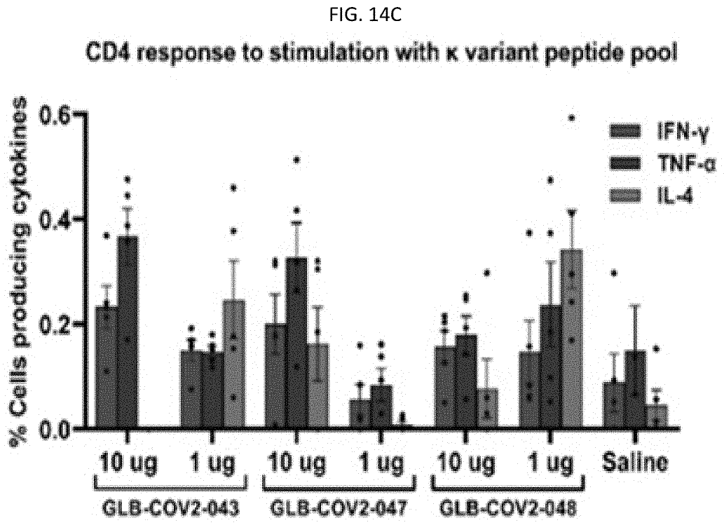

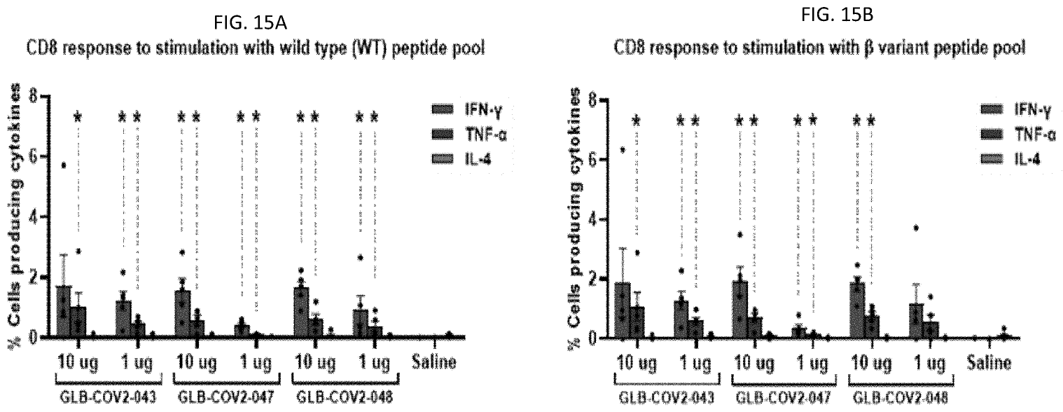

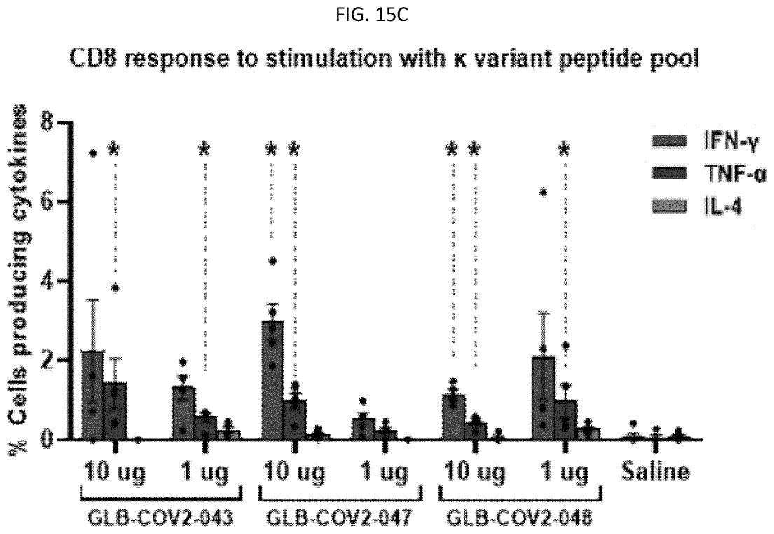

Individual data for stimulation with peptide pools WT, β, and κ are shown in FIGS. 14A, 14 B, and 14C (T CD4+) and FIGS. 15A, 15B, and 15C (T CD8+). All vaccine candidates enhanced the T CD4+and T CD8+production of Th1 cytokines (pro-inflammatory, antiviral) IFN-γ and TNF-α in percentage and did not significantly increase Th2 (anti-inflammatory cytokine IL-4 and IL-5 in both CD4+and CD8+cells). The results show that all vaccine candidates were biased toward the desirable Th1 T cell responses at the studied mRNA doses 1 μg and 10 μg when compared to the saline group. Mann-Whitney U tests were run, followed by Holm-S idak adjusted p-value for multiple comparisons. p-values <0.05 were considered statistically different, n=5.

### Example 8

### Studies with Vaccine Booster Shot

To measure the effect of a vaccine booster shot (third shot of an mRNA SARS-CoV-2 candidate) and the longitudinal neutralizing antibody levels in sera, blood samples isolated from wild type C57BL/6 mice, at several time points, immunized with the GLB mRNA vaccine candidate GLB-COV-2-043. Pseudovirus neutralization assay was used to access homologous and heterologous neutralizing antibodies to SARS-CoV-2 native and variant of concerns pseudotyped viruses to evaluate the Nab levels longitudinally.

The sera of mice vaccinated with 10 μg of GLB-COV-2-043 day 0 (prime), day 21 (boost), and a second boost on day 132 (four months from the first boosting injection) were tested at several time points (days 21, 42, 98, 132, 154, and 168) and compared to the saline group.

The experiments were carried out used a nonreplicating pseudovirus in the murine leukemic virus (MLV) backbone that expressed spike proteins from SARS-CoV-2 Wuhan, Omicron, Alpha, Beta, Delta, or Gamma variants. The pseudovirus virus has a luciferase reporter plasmid, which can be measured by luciferase emission when the particle enters a cell. The mice sera were incubated with pseudotyped virus for 1 hour and added to a HEKACE2 (cell expressing the human ACE2 receptor) cell line to measure the relative light units (RLU) given by the luciferase luminescence when the pseudoviral particles enter the cells. If the serum is highly neutralizing, the luciferase expression will be lower as the Pseudovirus will be blocked from infecting the HEKACE2 cells. The final 50% neutralizing titers (NT50) are calculated based on a non-linear regression curve fit using GraphPad Prism 9 software.

Neutralizing antibodies in serum samples isolated from mice were tested in the pseudovirus neutralization assay to measure functional neutralizing antibody responses against Omicron, Alpha, Gamma, Delta, Beta VOC, and Wuhan (wildtype).

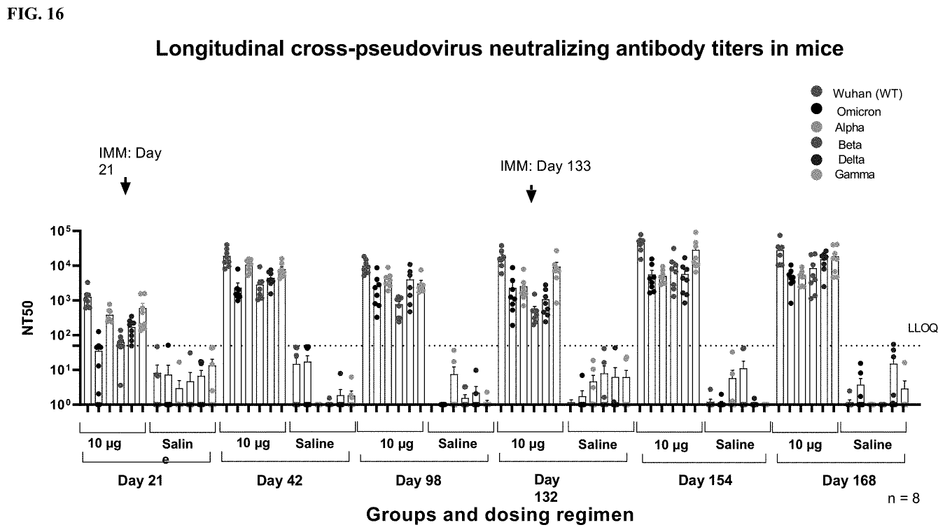

As shown in FIG. 16, GLB-COV-2-043 induced robust homologous and heterologous (VOC) levels of Nab at all time points. A third dose of GLB-COV-2-043 on day 133 enhanced the Nab response to all pseudoviruses tested, indicating that GLB-COV-2-043 induces long-term and anamnestic homologous and heterologous antibodies responses. The nAb titers were higher than the limit of quantification (LLOQ) and the saline group. The statistical analysis was performed by the non-parametric Kruskal-Wallis test, followed by Dunn's multiple comparisons test and adjustment of the p-value. p<0.05 was considered statistically different. The LLOQ for the assay was 50 (indicating the starting dilution for the assay was 1:50), n=8.

### Example 9

### ITS-HBG-Kozak 5′ UTR Supports High mRNA Titers Using In Vitro Transcription Reactions

A combinatorial library of 25 mRNA constructs encoding EGFP were designed and synthesized. The library consisted of all combinations of 5 sequences of 5′ UTR and 5 sequences of 3′ UTR, each flanking an open reading frame encoding EGFP, with a 3′ 100-nucleotide polyA tail encoded in the template followed by a unique BspQI site for linearization. All 5′ UTR sequences contained “AGG” initiator nucleotides for co-transcriptional capping with CleanCap AG reagent (TriLink BioTechnologies, Inc.). A summary of sequence designs is provided in Table 1.

Plasmid DNA templates encoding the mRNA library were propagated in E. coli strain DH10b, and plasmids recovered by Plasmid Midi Kit (Qiagen). Templates were linearized by BspQI digestion and recovered by phenol-chloroform extraction. RNAs were synthesized in 250 μL reactions using HiScribe™ T7 High Yield RNA Synthesis Kit (New England BioLabs) following manufacturer protocols and including 4 mM CleanCap AG reagent as well as 5 mM N1-methylpseudouridine triphosphate (m1ψTP) in place of UTP. Synthesis reactions were treated with DNase I (Aldevron), recovered by lithium chloride precipitation, resuspended in 250 μL nuclease-free water, and quantified by UV absorbance at 260 nm. Reactions were grouped by 5′ UTR sequence and analyzed via one-way ANOVA.

TABLE 1
Summary of sequence designs.

| UTR | Sequence |
|---|---|
| ITS (SEQ ID | aggagaccaggaauuacauuugcuucug |
| NO: 11)-HBG | acacaacuguguucacuagcaaccucaa |
| 5′-(SEQ ID NO: | acagagccgcc |
| 12)-Kozak (SEQ | (SEQ ID NO: 17) |
| ID NO: 13) | |
| 5′ AGG-HBA- | agggcgaacuaguacucuucuggucccca |
| Kozak | cagacucagagagaacgccacc |
| | (SEQ ID NO: 18) |
| 5′ AGG-HSD- | agggucccgcagucggcguccagcggcuc |
| Kozak | uucugcuuguucgugugugugucguugca |
| | ggccuuagccgcc |
| | (SEQ ID NO: 19) |
| 5′ AGG-MOD- | agggaaauaagagagaaaagaagaguaaga |
| Kozak | agaaauauaagagccacc |
| | (SEQ ID NO: 20) |
| 5′ AGG-NCA- | aggcaaaaaucaaaaucaaucaucaucaca |
| 7d-Kozak | acaucaacaaucaaucaucaacacaucauc |
| | aagacaccacc |
| | (SEQ ID NO: 21) |
| 3′ 2X HBG | agcucgcuuucuugcuguccaauuucuauu |
| | aaagguuccuuuguucccuaaguccaacua |
| | cuaaacugggggauauuaugaagggccuug |
| | agcaucuggauucugccuaauaagaaacau |
| | uuauugucauugcagcucgcuuucuugcug |
| | uccaauuucuauuaaagguuccuuuguucc |
| | cuaaguccaacuacuaaacugggggauauu |
| | augaagggccuugagcaucuggauucugcc |
| | uaauaagaaacauuuauugucauug |
| | (SEQ ID NO: 22) |
| 3′ AES-mtRNR1 | cugguacugcaugcacgcaaugcuagcugc |
| | cccuuucccguccuggguaccccgagucuc |
| | ccccgaccucgggucccagguaugcuccca |
| | ccuccaccugccccacucaccaccucugcu |
| | aguuccagacaccucccaagcacgcagcaa |
| | ugcagcucaaaacgcuuagccuagccacac |
| | ccccacgggaaacagcagugauuaaccuuu |
| | agcaauaaacgaaaguuuaacuaagcuaua |
| | cuaaccccaggguugguc |
| | aauuucgugccagccacacc |
| | (SEQ ID NO: 23) |
| 3′ ALB | caucacauuuaaaagcaucucagccuacca |
| | ugagaauaagagaaagaaaaugaagaucaa |
| | uagcuuauucaucucuuuuucuuuuucguu |
| | gguguaaagccaacacccugucuaaaaaac |
| | auaaauuucuuuaaucauuuugccucuugu |
| | cucugugcuccacucaccaaaaaauggaaa |
| | gaaccu |
| | (SEQ ID NO: 24) |
| 3′ HBA | ugauaauaggcuggagccucgguggccaug |
| | cuucuugccccuugggccuccccccagccc |
| | cuccuccccuuccugcacccguacccccgu |
| | ggucuuugaauaaagucuga |
| | (SEQ ID NO: 25) |
| 3′ S27a + R3U | uuguguaugcguuaauaaaaagaaggaacu |
| | cguaaaaacucaauguauuucugaggaagc |
| | guggugcauaaugccacgcagcgucugcau |
| | aacuuuuauuauuucuuuuauuaaucaaca |
| | aa |
| | (SEQ ID NO: 26) |

HBG = Human β-globin
HBA = Human α-globin
HSD = Hydroxy steroid dehydrogenase
NCA-7d = Synthetic
AES-mtRNR1 = Synthetic
ALB = Human albumin
MOD = Synthetic
S27a + R3U = Synthetic

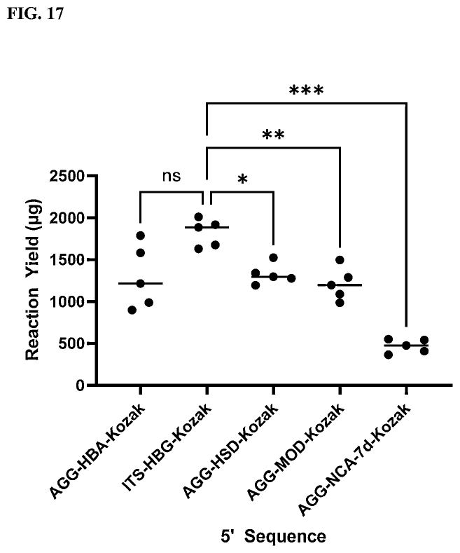

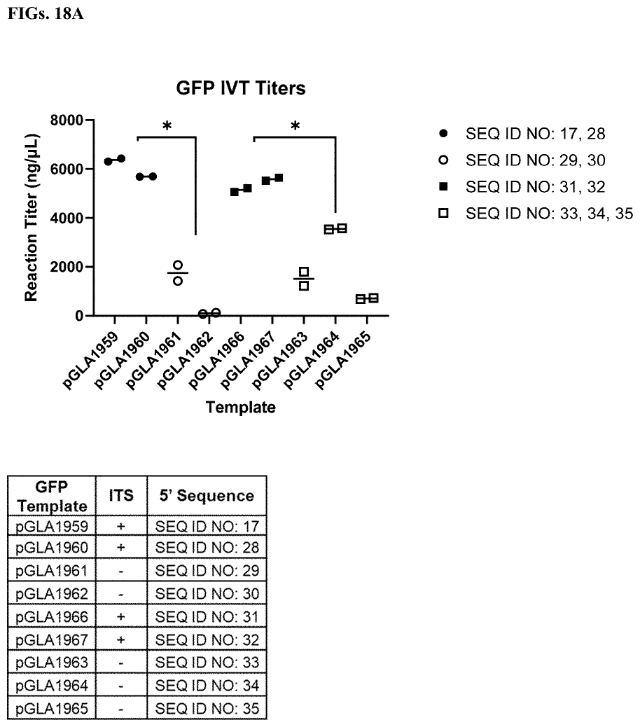

As shown in FIG. 17, mRNA synthesis yield was highly dependent on the sequence of the 5′ UTR. Of the five sequences tested, the ITS-HBG-Kozak-5′ UTR resulted in the highest reaction yields, approaching the theoretical maximum yield from the HiScribe™ kit (approximately 8.5 mg/mL or 2125 μg in a 250 μL reaction). Differences between reaction titer were significantly greater for sequences containing the 5′ HBG-ITS than all other 5′ UTRs, with the exception of HBA (P=0.069). These results suggest that inclusion of the 5′ ITS-HBG-Kozak UTR results in mRNA sequences that are particularly high-yielding under standard in vitro transcription conditions.

### Example 10

### ITS Increases In Vitro Transcription Yields in Multiple 5′ UTR and Open Reading Frame Sequence Contexts

The effect of the ITS on mRNA synthesis by in vitro transcription was assessed. Two 5′ UTR sequences (ITS-HBG-Kozak and AGG-NCA-7d-Kozak, representing high-yielding and low-yielding sequences) were selected based on the results from Example 9. A series of mRNAs were designed to assess the impact of the ITS on synthesis yields in the context of HBG and NCA-7d 5′ UTRs. A summary of the mRNA designs tested is shown in Table 2. Each of the mRNAs contained the same 3′ human beta-globin UTR, as well as a template-encoded polyAioo tail. To assess dependence on the coding sequence, the same series of mRNAs were designed encoding EGFP and the S protein from the Wuhan strain of SARS-CoV-2 coronavirus.

TABLE 2
Summary of the mRNA designs tested.

| GFP | SARS-CoV-2 | | | |
|---|---|---|---|---|
| Template | Template | ITS | 5′ UTR | 5′ Sequence (ITS in Caps) |
| pGLA1959 | pGLA1968 | SEQ ID | 5′ ITS- | AGGAGACCAGGAAUUacauuugcuucugac |
| | | NO: 9 | HBG- | acaacuguguucacuagcaaccucaaacagagccgcc |
| | | | Kozak | (SEQ ID NO: 17) |
| pGLA1960 | pGLA1969 | SEQ ID | 5′ ITS- | GGGAGACCAGGAAUUacauuugcuucugac |
| | | NO: 10 | HBG- | acaacuguguucacuagcaaccucaaacagagccgcc |
| | | | Kozak | (SEQ ID NO: 28) |
| pGLA1961 | pGLA1970 | — | 5′ AGG- | AGGacauuugcuucugacacaacuguguucacuagca |
| | | | HBG- | accucaaacagagccgcc (SEQ ID NO: 29) |
| | | | Kozak | |
| pGLA1962 | pGLA1971 | — | 5′ G- | Gacauuugcuucugacacaacuguguucacuagcaacc |
| | | | HBG- | ucaaacagagccgcc (SEQ ID NO: 30) |
| | | | Kozak | |
| pGLA1966 | pGLA1975 | SEQ ID | 5′ ITS- | AGGAGACCAGGAAUUggcaaaaaucaaaauc |
| | | NO: 9 | NCA-7d- | aaucaucaucacaacaucaacaaucaaucaucaacacauc |
| | | | Kozak | aucaagacaccacc (SEQ ID NO: 31) |
| pGLA1967 | pGLA1976 | SEQ ID | 5′ ITS- | GGGAGACCAGGAAUUggcaaaaaucaaaauc |
| | | NO: 10 | NCA-7d- | aaucaucaucacaacaucaacaaucaaucaucaacacauc |
| | | | Kozak | aucaagacaccacc (SEQ ID NO: 32) |
| PGLA1963 | PGLA1972 | — | AGG-5′ | AGGcaaaaaucaaaaucaaucaucaucacaacaucaac |
| | | | NCA-7d- | aaucaaucaucaacacaucaucaagacaccacc (SEQ |
| | | | Kozak | ID NO: 33) |
| PGLA1964 | pGLA1973 | — | 5′ GG- | GGcaaaaaucaaaaucaaucaucaucacaacaucaacaa |
| | | | NCA-7d- | ucaaucaucaacacaucaucaagacaccacc (SEQ ID |
| | | | Kozak | NO: 34) |
| PGLA1965 | pGLA1974 | — | 5′ GG- | Gcaaaaaucaaaaucaaucaucaucacaacaucaacaau |
| | | | NCA-7d- | caaucaucaacacaucaucaagacaccacc (SEQ ID |
| | | | Kozak | NO: 35) |

Template plasmids encoding the mRNAs described were constructed using Golden Gate assembly based on a high-copy E. coli plasmid backbone. As in Example 9, plasmid templates encoded a 3′ 100-nucleotide polyA tail followed by a unique BspQI site for linearization. Templates were propagated, recovered, and linearized as described in Example 9. mRNAs were synthesized in 100 μL in vitro transcription reactions by incubating T7 RNA polymerase, nucleoside triphosphates (ATP, GTP, CTP, and m1ψTP), and linearized template in a reaction buffer consisting of Tris-HCl and MgSO₄. Reactions synthesizing mRNAs with AGG initiating sequences also included 4 mM CleanCap AG reagent (TriLink BioTechnologies, Inc.). Reactions were incubated for 1 hour, then treated with DNase I prior to recovery by lithium chloride precipitation. Samples were resuspended in 100 μL nuclease-free water and quantified by UV absorbance at 260 nm. Reactions were grouped by 5′ UTR sequence and analyzed via one-way ANOVA.

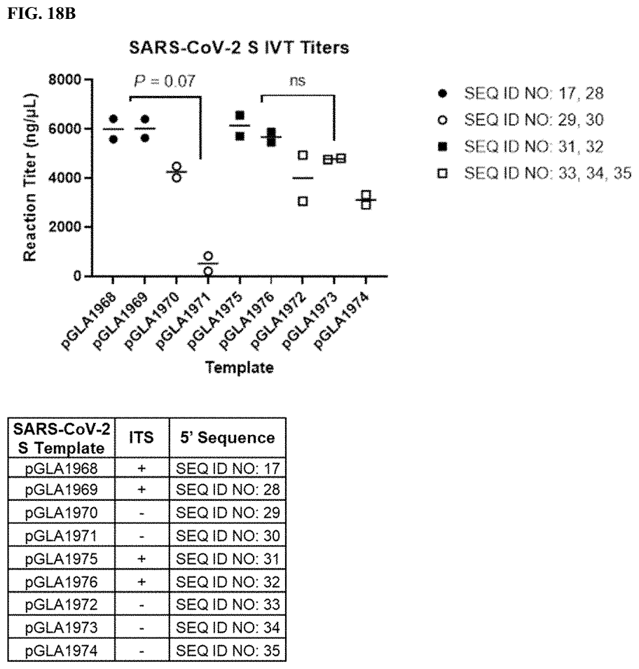

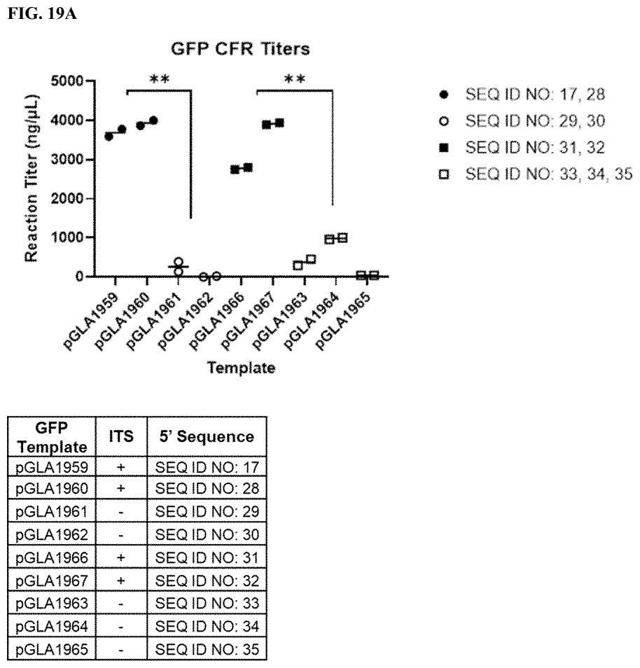

As shown in FIG. 18, reactions synthesizing mRNA molecules containing the ITS of SEQ ID NO: 9 or SEQ ID NO: 10 reached higher titers in all constructs contexts tested. For both HBG and NCA-7d 5′ UTRs, molecules with the AGG initiator reached approximately 3X and 1.5X higher titers in the context of GFP (FIG. 18A) and SARS-CoV-2 S (FIG. 18B) coding sequences, respectively, when the ITS was present. Similarly, synthesis reactions producing molecules containing the GGG ITS resulted in higher titers than molecules with GG or G initiators in all UTR and coding sequence contexts. Taken together, these results suggest the ITS of the present disclosure positively impacts mRNA titers and yields in in vitro transcription reactions.

### Example 11

### ITS Increases Transcription Yields in Cell-Free RNA Synthesis Reactions in Multiple 5′ UTR and Open Reading Frame Sequence Contexts

The effect of the ITS on yields in cell-free mRNA synthesis reactions was assessed. A series of mRNAs were synthesized in cell-free reactions using the linearized templates described in Example 10. Cell-free synthesis reactions similar to those described in WO 2020/205793 were used. For mRNAs with AGG initiating sequences, 5 mM CleanCap AG (TriLink BioTechnologies, Inc.) was also included in the reaction. DNase treatment, RNA recovery, and RNA quantification were performed as described for in vitro transcription reactions.

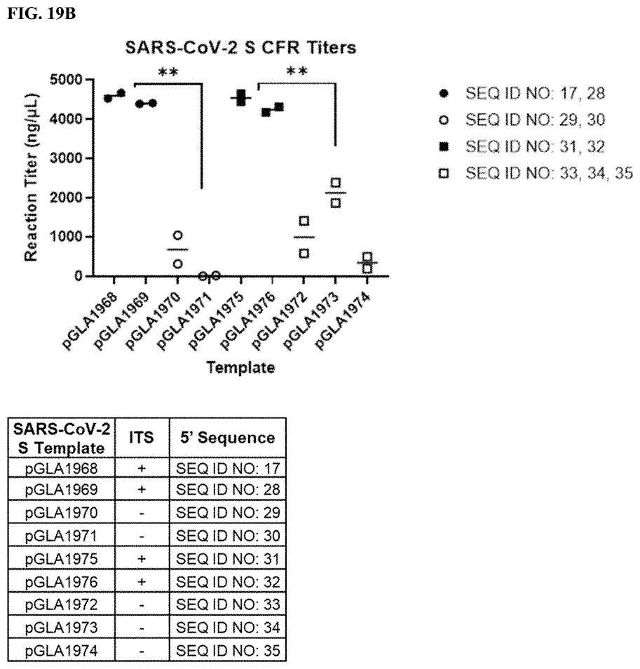

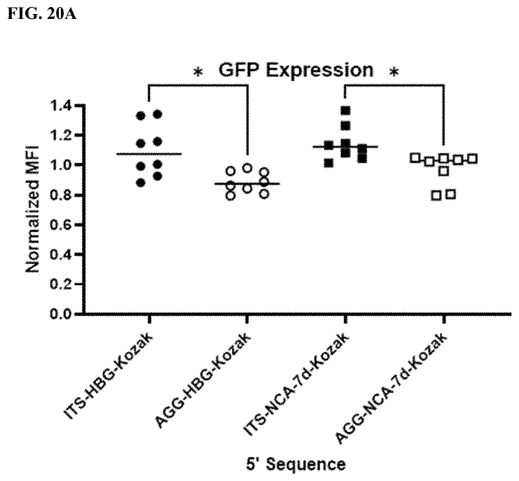

As shown in FIG. 19, reactions synthesizing mRNA molecules containing the ITS reached higher titers in all constructs contexts tested. For HBG 5′ UTRs, molecules with the AGG initiator reached approximately 14X and 7X higher titers in the context of GFP (FIG. 19A) and SARS-CoV-2 S (FIG. 19B) coding sequences, respectively, when the ITS was present. For NCA-7d 5′ UTRs, molecules with the AGG initiator reached approximately 7× and 5× higher titers in the context of GFP and SARS-CoV-2 S coding sequences, respectively, when the ITS was present. Similarly, synthesis reactions producing molecules containing the GGG ITS resulted in higher titers than molecules with GG or G initiators in all UTR and coding sequence contexts. Taken together, these results suggest that ITS positively impacts mRNA titers and yields in cell-free RNA synthesis reactions.

### Example 12

### ITS Preserves Translational Potency in Multiple 5′ UTR and Coding Sequence Contexts

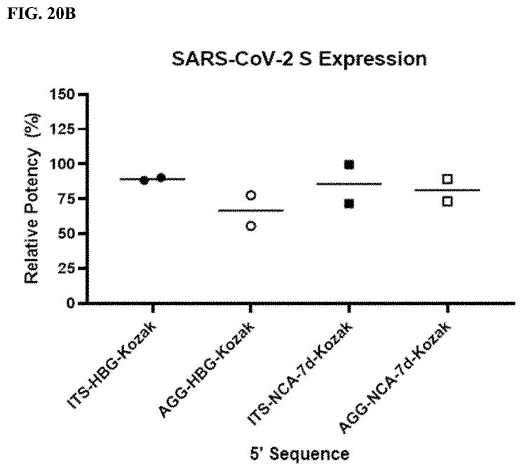

The molecules produced in Example 9 were analyzed for potency in cell-based assays by transfecting the mRNA into HEK293FT cells. For GFP potency assays, cells were transfected using Lipofectamine MessengerMAX™ (Thermo Fisher). Potency was assessed as median fluorescence intensity (MFI) of GFP-positive cells compared to a reference GFP mRNA. For spike potency assays, mRNA was transfected using Lipofectamine MessengerMAX (Thermo Fisher) and protein expression was quantified by ELISA. Analysis was performed using one-way ANOVA. As shown in FIGS. 20A and 20B, inclusion of the ITS sequence in the context of both HBG and NCA-7d 5′ UTRs preserved translational potency for both EGFP (FIG. 20A) and SARS-CoV-2 (FIG. 20B) spike-encoding mRNAs. FIG. 20A shows a statistically significant increase in expression of GFP in both the HBG-ITS and NCA-ITS compared to their respective versions without ITS. The results suggest that ITS can be used to improve mRNA manufacturability and may provide a potency benefit in some contexts.

SEQUENCES
Full length ORF (DNA template) encoding
SARS-CoV2 spike protein (Esp3I
linearized template)
SEQ ID NO: 1
atgttcgtgttcctggtgctgctgccgctggtgagcagccagtgcgtgaacctgacgacgcggacgcagctgc
cgccggcgtacacgaacagcttcacgcggggggtgtactacccggacaaggtgttccggagcagcgtgctgca
cagcacgcaggacctgttcctgccgttcttcagcaacgtgacgtggttccacgcgatccacgtgagcgggacg
aacgggacgaagcggttcgacaacccggtgctgccgttcaacgacggggtgtacttcgcgagcacggagaaga
gcaacatcatccgggggtggatcttcgggacgacgctggacagcaagacgcagagcctgctgatcgtgaacaa
cgcgacgaacgtggtgatcaaggtgtgcgagttccagttctgcaacgacccgttcctgggggtgtactaccac
aagaacaacaagagctggatggagagcgagttccgggtgtacagcagcgcgaacaactgcacgttcgagtacg
tgagccagccgttcctgatggacctggaggggaagcaggggaacttcaagaacctgcgggagttcgtgttcaa
gaacatcgacgggtacttcaagatctacagcaagcacacgccgatcaacctggtgcgggacctgccgcagggg
ttcagcgcgctggagccgctggtggacctgccgatcgggatcaacatcacgcggttccagacgctgctggcgc
tgcaccggagctacctgacgccgggggacagcagcagcgggtggacggcgggggcggcggcgtactacgtggg
gtacctgcagccgcggacgttcctgctgaagtacaacgagaacgggacgatcacggacgcggtggactgcgcg
ctggacccgctgagcgagaccaagtgcacgctgaagagcttcacggtggagaaggggatctaccagacgagca
acttccgggtgcagccgacggagagcatcgtgcggttcccgaacatcacgaacctgtgcccgttcggggaggt
gttcaacgcgacgcggttcgcgagcgtgtacgcgtggaaccggaagcggatcagcaactgcgtggcggactac
agcgtgctgtacaacagcgcgagcttcagcacgttcaagtgctacggggtgagcccgacgaagctgaacgacc
tgtgcttcacgaacgtgtacgcggacagcttcgtgatccggggggacgaggtgcggcagatcgcgccggggca
gacggggaagatcgcggactacaactacaagctgccggacgacttcacggggtgcgtgatcgcgtggaacagc
aacaacctggacagcaaggtgggggggaactacaactacctgtaccggctgttccggaagagcaacctgaagc
cgttcgagcgggacatcagcacggagatctaccaggcggggagcacgccgtgcaacggggtggaggggttcaa
ctgctacttcccgctgcagagctacgggttccagccgacgaacggggtggggtaccagccgtaccgggtggtg
gtgctgagcttcgagctgctgcacgcgccggcgacggtgtgcgggccgaagaagagcacgaacctggtgaaga
acaagtgcgtgaacttcaacttcaacgggctgacggggacgggggtgctgacggagagcaacaagaagttcct
gccgttccagcagttcgggcgggacatcgcggacacgacggacgcggtgcgggacccgcagacgctggagatc
ctggacatcacgccgtgcagcttcgggggggtgagcgtgatcacgccggggacgaacacgagcaaccaggtgg
cggtgctgtaccaggacgtgaactgcacggaggtgccggtggcgatccacgcggaccagctgacgccgacgtg
gcgggtgtacagcacggggagcaacgtgttccagacgcgggcggggtgcctgatcggggcggagcacgtgaac
aacagctacgagtgcgacatcccgatcggggcggggatctgcgcgagctaccagacgcagacgaacagcccgc
ggcgggcgcggagcgtggcgagccagagcatcatcgcgtacacgatgagcctgggggcggagaacagcgtggc
gtacagcaacaacagcatcgcgatcccgacgaacttcacgatcagcgtgacgacggagatcctgccggtgagc
atgacgaagacgagcgtggactgcacgatgtacatctgcggggacagcacggagtgcagcaacctgctgctgc
agtacgggagcttctgcacgcagctgaaccgggcgctgacggggatcgcggtggagcaggacaagaacacgca
ggaggtgttcgcgcaggtgaagcagatctacaagacgccgccgatcaaggacttcggggggttcaacttcagc
cagatcctgccggacccgagcaagccgagcaagcggagcttcatcgaggacctgctgttcaacaaggtgacgc
tggcggacgcggggttcatcaagcagtacggggactgcctgggggacatcgcggcgcgggacctgatctgcgc
gcagaagttcaacgggctgacggtgctgccgccgctgctgacggacgagatgatcgcgcagtacacgagcgcg
ctgctggcggggacgatcacgagcgggtggacgttcggggcgggggcggcgctgcagatcccgttcgcgatgc
agatggcgtaccggttcaacgggatcggggtgacgcagaacgtgctgtacgagaaccagaagctgatcgcgaa
ccagttcaacagcgcgatcgggaagatccaggacagcctgagcagcacggcgagcgcgctggggaagctgcag
gacgtggtgaaccagaacgcgcaggcgctgaacacgctggtgaagcagctgagcagcaacttcggggcgatca
gcagcgtgctgaacgacatcctgagccggctggacaaggtggaggcggaggtgcagatcgaccggctgatcac
ggggcggctgcagagcctgcagacgtacgtgacgcagcagctgatccgggcggcggagatccgggcgagcgcg
aacctggcggcgacgaagatgagcgagtgcgtgctggggcagagcaagcgggtggacttctgcgggaaggggt
accacctgatgagcttcccgcagagcgcgccgcacggggtggtgttcctgcacgtgacgtacgtgccggcgca
ggagaagaacttcacgacggcgccggcgatctgccacgacgggaaggcgcacttcccgcgggagggggtgttc
gtgagcaacgggacgcactggttcgtgacgcagcggaacttctacgagccgcagatcatcacgacggacaaca
cgttcgtgagcgggaactgcgacgtggtgatcgggatcgtgaacaacacggtgtacgacccgctgcagccgga
gctggacagcttcaaggaggagctggacaagtacttcaagaaccacacgagcccggacgtggacctgggggac
atcagcgggatcaacgcgagcgtggtgaacatccagaaggagatcgaccggctgaacgaggtggcgaagaacc
tgaacgagagcctgatcgacctgcaggagctggggaagtacgagcagtacatcaagtggccgtggtacatctg
gctggggttcatcgcggggctgatcgcgatcgtgatggtgacgatcatgctgtgctgcatgacgagctgctgc
agctgcctgaaggggtgctgcagctgcgggagctgctgcaagttcgacgaggacgacagcgagccggtgctga
agggggtgaagctgcactacacgtga
Full length ORF (RNA) encoding
SARS-CoV2 spike protein (from Esp3I
linearized template)
SEQ ID NO: 2
auguucguguuccuggugcugcugccgcuggugagcagccagugcgugaaccugacgacgcggacgcagcugc
cgccggcguacacgaacagcuucacgcgggggguguacuacccggacaagguguuccggagcagcgugcugca
cagcacgcaggaccuguuccugccguucuucagcaacgugacgugguuccacgcgauccacgugagcgggacg
aacgggacgaagcgguucgacaacccggugcugccguucaacgacgggguguacuucgcgagcacggagaaga
gcaacaucauccggggguggaucuucgggacgacgcuggacagcaagacgcagagccugcugaucgugaacaa
cgcgacgaacguggugaucaaggugugcgaguuccaguucugcaacgacccguuccuggggguguacuaccac
aagaacaacaagagcuggauggagagcgaguuccggguguacagcagcgcgaacaacugcacguucgaguacg
ugagccagccguuccugauggaccuggaggggaagcaggggaacuucaagaaccugcgggaguucguguucaa
gaacaucgacggguacuucaagaucuacagcaagcacacgccgaucaaccuggugcgggaccugccgcagggg
uucagcgcgcuggagccgcugguggaccugccgaucgggaucaacaucacgcgguuccagacgcugcuggcgc
ugcaccggagcuaccugacgccgggggacagcagcagcggguggacggcgggggcggcggcguacuacguggg
guaccugcagccgcggacguuccugcugaaguacaacgagaacgggacgaucacggacgcgguggacugcgcg
cuggacccgcugagcgagaccaagugcacgcugaagagcuucacgguggagaaggggaucuaccagacgagca
acuuccgggugcagccgacggagagcaucgugcgguucccgaacaucacgaaccugugcccguucggggaggu
guucaacgcgacgcgguucgcgagcguguacgcguggaaccggaagcggaucagcaacugcguggcggacuac
agcgugcuguacaacagcgcgagcuucagcacguucaagugcuacggggugagcccgacgaagcugaacgacc
ugugcuucacgaacguguacgcggacagcuucgugauccggggggacgaggugcggcagaucgcgccggggca
gacggggaagaucgcggacuacaacuacaagcugccggacgacuucacggggugcgugaucgcguggaacagc
aacaaccuggacagcaaggugggggggaacuacaacuaccuguaccggcuguuccggaagagcaaccugaagc
cguucgagcgggacaucagcacggagaucuaccaggcggggagcacgccgugcaacgggguggagggguucaa
cugcuacuucccgcugcagagcuacggguuccagccgacgaacggggugggguaccagccguaccggguggug
gugcugagcuucgagcugcugcacgcgccggcgacggugugcgggccgaagaagagcacgaaccuggugaaga
acaagugcgugaacuucaacuucaacgggcugacggggacgggggugcugacggagagcaacaagaaguuccu
gccguuccagcaguucgggcgggacaucgcggacacgacggacgcggugcgggacccgcagacgcuggagauc
cuggacaucacgccgugcagcuucgggggggugagcgugaucacgccggggacgaacacgagcaaccaggugg
cggugcuguaccaggacgugaacugcacggaggugccgguggcgauccacgcggaccagcugacgccgacgug
gcggguguacagcacggggagcaacguguuccagacgcgggcggggugccugaucggggcggagcacgugaac
aacagcuacgagugcgacaucccgaucggggcggggaucugcgcgagcuaccagacgcagacgaacagcccgc
ggcgggcgcggagcguggcgagccagagcaucaucgcguacacgaugagccugggggcggagaacagcguggc
guacagcaacaacagcaucgcgaucccgacgaacuucacgaucagcgugacgacggagauccugccggugagc
augacgaagacgagcguggacugcacgauguacaucugcggggacagcacggagugcagcaaccugcugcugc
aguacgggagcuucugcacgcagcugaaccgggcgcugacggggaucgcgguggagcaggacaagaacacgca
ggagguguucgcgcaggugaagcagaucuacaagacgccgccgaucaaggacuucgggggguucaacuucagc
cagauccugccggacccgagcaagccgagcaagcggagcuucaucgaggaccugcuguucaacaaggugacgc
uggcggacgcgggguucaucaagcaguacggggacugccugggggacaucgcggcgcgggaccugaucugcgc
gcagaaguucaacgggcugacggugcugccgccgcugcugacggacgagaugaucgcgcaguacacgagcgcg
cugcuggcggggacgaucacgagcggguggacguucggggcgggggcggcgcugcagaucccguucgcgaugc
agauggcguaccgguucaacgggaucggggugacgcagaacgugcuguacgagaaccagaagcugaucgcgaa
ccaguucaacagcgcgaucgggaagauccaggacagccugagcagcacggcgagcgcgcuggggaagcugcag
gacguggugaaccagaacgcgcaggcgcugaacacgcuggugaagcagcugagcagcaacuucggggcgauca
gcagcgugcugaacgacauccugagccggcuggacaagguggaggcggaggugcagaucgaccggcugaucac
ggggcggcugcagagccugcagacguacgugacgcagcagcugauccgggcggcggagauccgggcgagcgcg
aaccuggcggcgacgaagaugagcgagugcgugcuggggcagagcaagcggguggacuucugcgggaaggggu
accaccugaugagcuucccgcagagcgcgccgcacggggugguguuccugcacgugacguacgugccggcgca
ggagaagaacuucacgacggcgccggcgaucugccacgacgggaaggcgcacuucccgcgggaggggguguuc
gugagcaacgggacgcacugguucgugacgcagcggaacuucuacgagccgcagaucaucacgacggacaaca
cguucgugagcgggaacugcgacguggugaucgggaucgugaacaacacgguguacgacccgcugcagccgga
gcuggacagcuucaaggaggagcuggacaaguacuucaagaaccacacgagcccggacguggaccugggggac
aucagcgggaucaacgcgagcguggugaacauccagaaggagaucgaccggcugaacgagguggcgaagaacc
ugaacgagagccugaucgaccugcaggagcuggggaaguacgagcaguacaucaaguggccgugguacaucug
gcugggguucaucgcggggcugaucgcgaucgugauggugacgaucaugcugugcugcaugacgagcugcugc
agcugccugaaggggugcugcagcugcgggagcugcugcaaguucgacgaggacgacagcgagccggugcuga
agggggugaagcugcacuacacguga
Full length ORF (DNA template) encoding
SARS-CoV2 spike protein
(BspQI linearized template)
SEQ ID NO: 3
atgttcgtgttcctggtgctgctgccgctggtgagcagccagtgcgtgaacctgacgacgcggacgcagctgc
cgccggcgtacacgaacagcttcacgcggggggtgtactacccggacaaggtgttccggagcagcgtgctgca
cagcacgcaggacctgttcctgccgttcttcagcaacgtgacgtggttccacgcgatccacgtgagcgggacg
aacgggacgaagcggttcgacaacccggtgctgccgttcaacgacggggtgtacttcgcgagcacggagaaga
gtaacatcatccgggggtggatcttcgggacgacgctggacagcaagacgcagagcctgctgatcgtgaacaa
cgcgacgaacgtggtgatcaaggtgtgcgagttccagttctgcaacgacccgttcctgggggtgtactaccac
aagaacaacaagagctggatggagagcgagttccgggtgtacagcagcgcgaacaactgcacgttcgagtacg
tgagccagccgttcctgatggacctggaggggaagcaggggaacttcaagaacctgcgggagttcgtgttcaa
gaacatcgacgggtacttcaagatctacagcaagcacacgccgatcaacctggtgcgggacctgccgcagggg
ttcagcgcgctggagccgctggtggacctgccgatcgggatcaacatcacgcggttccagacgctgctggcgc
tgcaccggagctacctgacgccgggggacagcagcagcgggtggacggcgggggcggcggcgtactacgtggg
gtacctgcagccgcggacgttcctgctgaagtacaacgagaacgggacgatcacggacgcggtggactgcgcg
ctggacccgctgagcgagaccaagtgcacgctgaagagtttcacggtggagaaggggatctaccagacgagca
acttccgggtgcagccgacggagagcatcgtgcggttcccgaacatcacgaacctgtgcccgttcggggaggt
gttcaacgcgacgcggttcgcgagcgtgtacgcgtggaaccggaagcggatcagcaactgcgtggcggactac
agcgtgctgtacaacagcgcgagcttcagcacgttcaagtgctacggggtgagcccgacgaagctgaacgacc
tgtgcttcacgaacgtgtacgcggacagcttcgtgatccggggggacgaggtgcggcagatcgcgccggggca
gacggggaagatcgcggactacaactacaagctgccggacgacttcacggggtgcgtgatcgcgtggaacagc
aacaacctggacagcaaggtgggggggaactacaactacctgtaccggctgttccggaagagtaacctgaagc
cgttcgagcgggacatcagcacggagatctaccaggcggggagcacgccgtgcaacggggtggaggggttcaa
ctgctacttcccgctgcagagctacgggttccagccgacgaacggggtggggtaccagccgtaccgggtggtg
gtgctgagcttcgagctgctgcacgcgccggcgacggtgtgcgggccgaagaagagtacgaacctggtgaaga
acaagtgcgtgaacttcaacttcaacgggctgacggggacgggggtgctgacggagagcaacaagaagttcct
gccgttccagcagttcgggcgggacatcgcggacacgacggacgcggtgcgggacccgcagacgctggagatc
ctggacatcacgccgtgcagcttcgggggggtgagcgtgatcacgccggggacgaacacgagcaaccaggtgg
cggtgctgtaccaggacgtgaactgcacggaggtgccggtggcgatccacgcggaccagctgacgccgacgtg
gcgggtgtacagcacggggagcaacgtgttccagacgcgggcggggtgcctgatcggggcggagcacgtgaac
aacagctacgagtgcgacatcccgatcggggcggggatctgcgcgagctaccagacgcagacgaacagcccgc
ggcgggcgcggagcgtggcgagccagagcatcatcgcgtacacgatgagcctgggggcggagaacagcgtggc
gtacagcaacaacagcatcgcgatcccgacgaacttcacgatcagcgtgacgacggagatcctgccggtgagc
atgacgaagacgagcgtggactgcacgatgtacatctgcggggacagcacggagtgcagcaacctgctgctgc
agtacgggagcttctgcacgcagctgaaccgggcgctgacggggatcgcggtggagcaggacaagaacacgca
ggaggtgttcgcgcaggtgaagcagatctacaagacgccgccgatcaaggacttcggggggttcaacttcagc
cagatcctgccggacccgagcaagccgagcaagcggagcttcatcgaggacctgctgttcaacaaggtgacgc
tggcggacgcggggttcatcaagcagtacggggactgcctgggggacatcgcggcgcgggacctgatctgcgc
gcagaagttcaacgggctgacggtgctgccgccgctgctgacggacgagatgatcgcgcagtacacgagcgcg
ctgctggcggggacgatcacgagcgggtggacgttcggggcgggggcggcgctgcagatcccgttcgcgatgc
agatggcgtaccggttcaacgggatcggggtgacgcagaacgtgctgtacgagaaccagaagctgatcgcgaa
ccagttcaacagcgcgatcgggaagatccaggacagcctgagcagcacggcgagcgcgctggggaagctgcag
gacgtggtgaaccagaacgcgcaggcgctgaacacgctggtgaagcagctgagcagcaacttcggggcgatca
gcagcgtgctgaacgacatcctgagccggctggacaaggtggaggcggaggtgcagatcgaccggctgatcac
ggggcggctgcagagcctgcagacgtacgtgacgcagcagctgatccgggcggcggagatccgggcgagcgcg
aacctggcggcgacgaagatgagcgagtgcgtgctggggcagagcaagcgggtggacttctgcgggaaggggt
accacctgatgagcttcccgcagagcgcgccgcacggggtggtgttcctgcacgtgacgtacgtgccggcgca
ggagaagaacttcacgacggcgccggcgatctgccacgacgggaaggcgcacttcccgcgggagggggtgttc
gtgagcaacgggacgcactggttcgtgacgcagcggaacttctacgagccgcagatcatcacgacggacaaca
cgttcgtgagcgggaactgcgacgtggtgatcgggatcgtgaacaacacggtgtacgacccgctgcagccgga
gctggacagcttcaaggaggagctggacaagtacttcaagaaccacacgagcccggacgtggacctgggggac
atcagcgggatcaacgcgagcgtggtgaacatccagaaggagatcgaccggctgaacgaggtggcgaagaacc
tgaacgagagcctgatcgacctgcaggagctggggaagtacgagcagtacatcaagtggccgtggtacatctg
gctggggttcatcgcggggctgatcgcgatcgtgatggtgacgatcatgctgtgctgcatgacgagctgctgc
agctgcctgaaggggtgctgcagctgcgggagctgctgcaagttcgacgaggacgacagcgagccggtgctga
agggggtgaagctgcactacacgtga
Full length ORF (RNA) encoding SARS-CoV2
spike protein (BspQI
linearized template)
SEQ ID NO: 4
auguucguguuccuggugcugcugccgcuggugagcagccagugcgugaaccugacgacgcggacgcagcugc
cgccggcguacacgaacagcuucacgcgggggguguacuacccggacaagguguuccggagcagcgugcugca
cagcacgcaggaccuguuccugccguucuucagcaacgugacgugguuccacgcgauccacgugagcgggacg
aacgggacgaagcgguucgacaacccggugcugccguucaacgacgggguguacuucgcgagcacggagaaga
guaacaucauccggggguggaucuucgggacgacgcuggacagcaagacgcagagccugcugaucgugaacaa
cgcgacgaacguggugaucaaggugugcgaguuccaguucugcaacgacccguuccuggggguguacuaccac
aagaacaacaagagcuggauggagagcgaguuccggguguacagcagcgcgaacaacugcacguucgaguacg
ugagccagccguuccugauggaccuggaggggaagcaggggaacuucaagaaccugcgggaguucguguucaa
gaacaucgacggguacuucaagaucuacagcaagcacacgccgaucaaccuggugcgggaccugccgcagggg
uucagcgcgcuggagccgcugguggaccugccgaucgggaucaacaucacgcgguuccagacgcugcuggcgc
ugcaccggagcuaccugacgccgggggacagcagcagcggguggacggcgggggcggcggcguacuacguggg
guaccugcagccgcggacguuccugcugaaguacaacgagaacgggacgaucacggacgcgguggacugcgcg
cuggacccgcugagcgagaccaagugcacgcugaagaguuucacgguggagaaggggaucuaccagacgagca
acuuccgggugcagccgacggagagcaucgugcgguucccgaacaucacgaaccugugcccguucggggaggu
guucaacgcgacgcgguucgcgagcguguacgcguggaaccggaagcggaucagcaacugcguggcggacuac
agcgugcuguacaacagcgcgagcuucagcacguucaagugcuacggggugagcccgacgaagcugaacgacc
ugugcuucacgaacguguacgcggacagcuucgugauccggggggacgaggugcggcagaucgcgccggggca
gacggggaagaucgcggacuacaacuacaagcugccggacgacuucacggggugcgugaucgcguggaacagc
aacaaccuggacagcaaggugggggggaacuacaacuaccuguaccggcuguuccggaagaguaaccugaagc
cguucgagcgggacaucagcacggagaucuaccaggcggggagcacgccgugcaacgggguggagggguucaa
cugcuacuucccgcugcagagcuacggguuccagccgacgaacggggugggguaccagccguaccggguggug
gugcugagcuucgagcugcugcacgcgccggcgacggugugcgggccgaagaagaguacgaaccuggugaaga
acaagugcgugaacuucaacuucaacgggcugacggggacgggggugcugacggagagcaacaagaaguuccu
gccguuccagcaguucgggcgggacaucgcggacacgacggacgcggugcgggacccgcagacgcuggagauc
cuggacaucacgccgugcagcuucgggggggugagcgugaucacgccggggacgaacacgagcaaccaggugg
cggugcuguaccaggacgugaacugcacggaggugccgguggcgauccacgcggaccagcugacgccgacgug
gcggguguacagcacggggagcaacguguuccagacgcgggcggggugccugaucggggcggagcacgugaac
aacagcuacgagugcgacaucccgaucggggcggggaucugcgcgagcuaccagacgcagacgaacagcccgc
ggcgggcgcggagcguggcgagccagagcaucaucgcguacacgaugagccugggggcggagaacagcguggc
guacagcaacaacagcaucgcgaucccgacgaacuucacgaucagcgugacgacggagauccugccggugagc
augacgaagacgagcguggacugcacgauguacaucugcggggacagcacggagugcagcaaccugcugcugc
aguacgggagcuucugcacgcagcugaaccgggcgcugacggggaucgcgguggagcaggacaagaacacgca
ggagguguucgcgcaggugaagcagaucuacaagacgccgccgaucaaggacuucgggggguucaacuucagc
cagauccugccggacccgagcaagccgagcaagcggagcuucaucgaggaccugcuguucaacaaggugacgc
uggcggacgcgggguucaucaagcaguacggggacugccugggggacaucgcggcgcgggaccugaucugcgc
gcagaaguucaacgggcugacggugcugccgccgcugcugacggacgagaugaucgcgcaguacacgagcgcg
cugcuggcggggacgaucacgagcggguggacguucggggcgggggcggcgcugcagaucccguucgcgaugc
agauggcguaccgguucaacgggaucggggugacgcagaacgugcuguacgagaaccagaagcugaucgcgaa
ccaguucaacagcgcgaucgggaagauccaggacagccugagcagcacggcgagcgcgcuggggaagcugcag
gacguggugaaccagaacgcgcaggcgcugaacacgcuggugaagcagcugagcagcaacuucggggcgauca
gcagcgugcugaacgacauccugagccggcuggacaagguggaggcggaggugcagaucgaccggcugaucac
ggggcggcugcagagccugcagacguacgugacgcagcagcugauccgggcggcggagauccgggcgagcgcg
aaccuggcggcgacgaagaugagcgagugcgugcuggggcagagcaagcggguggacuucugcgggaaggggu
accaccugaugagcuucccgcagagcgcgccgcacggggugguguuccugcacgugacguacgugccggcgca
ggagaagaacuucacgacggcgccggcgaucugccacgacgggaaggcgcacuucccgcgggaggggguguuc
gugagcaacgggacgcacugguucgugacgcagcggaacuucuacgagccgcagaucaucacgacggacaaca
cguucgugagcgggaacugcgacguggugaucgggaucgugaacaacacgguguacgacccgcugcagccgga
gcuggacagcuucaaggaggagcuggacaaguacuucaagaaccacacgagcccggacguggaccugggggac
aucagcgggaucaacgcgagcguggugaacauccagaaggagaucgaccggcugaacgagguggcgaagaacc
ugaacgagagccugaucgaccugcaggagcuggggaaguacgagcaguacaucaaguggccgugguacaucug
gcugggguucaucgcggggcugaucgcgaucgugauggugacgaucaugcugugcugcaugacgagcugcugc
agcugccugaaggggugcugcagcugcgggagcugcugcaaguucgacgaggacgacagcgagccggugcuga
agggggugaagcugcacuacacguga
SARS-CoV2 spike protein amino
acid sequence (GenBank: OHD43416.1)
SEQ ID NO: 5
MFVFLVLLPLVSSQCVNLTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFGEVFNATRFASVYAWNRKRISNCVADY
SVLYNSASFSTFKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPTNGVGYQPYRVV
VLSFELLHAPATVCGPKKSTNLVKNKCVNFNFNGLTGTGVLTESNKKFLPFQQFGRDIADTTDAVRDPQTLEI
LDITPCSFGGVSVITPGTNTSNQVAVLYQDVNCTEVPVAIHADQLTPTWRVYSTGSNVFQTRAGCLIGAEHVN
NSYECDIPIGAGICASYQTQTNSPRRARSVASQSIIAYTMSLGAENSVAYSNNSIAIPTNFTISVTTEILPVS
MTKTSVDCTMYICGDSTECSNLLLQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQKFNGLTVLPPLLTDEMIAQYTSA
LLAGTITSGWTFGAGAALQIPFAMQMAYRFNGIGVTQNVLYENQKLIANQFNSAIGKIQDSLSSTASALGKLQ
DVVNQNAQALNTLVKQLSSNFGAISSVLNDILSRLDKVEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPELDSFKEELDKYFKNHTSPDVDLGD
ISGINASVVNIQKEIDRLNEVAKNLNESLIDLQELGKYEQYIKWPWYIWLGFIAGLIAIVMVTIMLCCMTSCC
SCLKGCCSCGSCCKFDEDDSEPVLKGVKLHYT
mRNA sequence encoding SARS-CoV2 spike protein (Esp3I linearized
template), and including non-coding regions
(ITS, 5′ UTR, [symbol] [symbol] , 3′ UTR)
SEQ ID NO: 6
m7Gaggagaccaggaauuacauuugcuucugacacaacuguguucacuagcaaccucaaacaga [symbol] caug
uucguguuccuggugcugcugccgcuggugagcagccagugcgugaaccugacgacgcggacgcagcugccgc
cggcguacacgaacagcuucacgcgggggguguacuacccggacaagguguuccggagcagcgugcugcacag
cacgcaggaccuguuccugccguucuucagcaacgugacgugguuccacgcgauccacgugagcgggacgaac
gggacgaagcgguucgacaacccggugcugccguucaacgacgggguguacuucgcgagcacggagaagagca
acaucauccggggguggaucuucgggacgacgcuggacagcaagacgcagagccugcugaucgugaacaacgc
gacgaacguggugaucaaggugugcgaguuccaguucugcaacgacccguuccuggggguguacuaccacaag
aacaacaagagcuggauggagagcgaguuccggguguacagcagcgcgaacaacugcacguucgaguacguga
gccagccguuccugauggaccuggaggggaagcaggggaacuucaagaaccugcgggaguucguguucaagaa
caucgacggguacuucaagaucuacagcaagcacacgccgaucaaccuggugcgggaccugccgcagggguuc
agcgcgcuggagccgcugguggaccugccgaucgggaucaacaucacgcgguuccagacgcugcuggcgcugc
accggagcuaccugacgccgggggacagcagcagcggguggacggcgggggcggcggcguacuacguggggua
ccugcagccgcggacguuccugcugaaguacaacgagaacgggacgaucacggacgcgguggacugcgcgcug
gacccgcugagcgagaccaagugcacgcugaagagcuucacgguggagaaggggaucuaccagacgagcaacu
uccgggugcagccgacggagagcaucgugcgguucccgaacaucacgaaccugugcccguucggggagguguu
caacgcgacgcgguucgcgagcguguacgcguggaaccggaagcggaucagcaacugcguggcggacuacagc
gugcuguacaacagcgcgagcuucagcacguucaagugcuacggggugagcccgacgaagcugaacgaccugu
gcuucacgaacguguacgcggacagcuucgugauccggggggacgaggugcggcagaucgcgccggggcagac
ggggaagaucgcggacuacaacuacaagcugccggacgacuucacggggugcgugaucgcguggaacagcaac
aaccuggacagcaaggugggggggaacuacaacuaccuguaccggcuguuccggaagagcaaccugaagccgu
ucgagcgggacaucagcacggagaucuaccaggcggggagcacgccgugcaacgggguggagggguucaacug
cuacuucccgcugcagagcuacggguuccagccgacgaacggggugggguaccagccguaccgggugguggug
cugagcuucgagcugcugcacgcgccggcgacggugugcgggccgaagaagagcacgaaccuggugaagaaca
agugcgugaacuucaacuucaacgggcugacggggacgggggugcugacggagagcaacaagaaguuccugcc
guuccagcaguucgggcgggacaucgcggacacgacggacgcggugcgggacccgcagacgcuggagauccug
gacaucacgccgugcagcuucgggggggugagcgugaucacgccggggacgaacacgagcaaccagguggcgg
ugcuguaccaggacgugaacugcacggaggugccgguggcgauccacgcggaccagcugacgccgacguggcg
gguguacagcacggggagcaacguguuccagacgcgggcggggugccugaucggggcggagcacgugaacaac
agcuacgagugcgacaucccgaucggggcggggaucugcgcgagcuaccagacgcagacgaacagcccgcggc
gggcgcggagcguggcgagccagagcaucaucgcguacacgaugagccugggggcggagaacagcguggcgua
cagcaacaacagcaucgcgaucccgacgaacuucacgaucagcgugacgacggagauccugccggugagcaug
acgaagacgagcguggacugcacgauguacaucugcggggacagcacggagugcagcaaccugcugcugcagu
acgggagcuucugcacgcagcugaaccgggcgcugacggggaucgcgguggagcaggacaagaacacgcagga
gguguucgcgcaggugaagcagaucuacaagacgccgccgaucaaggacuucgggggguucaacuucagccag
auccugccggacccgagcaagccgagcaagcggagcuucaucgaggaccugcuguucaacaaggugacgcugg
cggacgcgggguucaucaagcaguacggggacugccugggggacaucgcggcgcgggaccugaucugcgcgca
gaaguucaacgggcugacggugcugccgccgcugcugacggacgagaugaucgcgcaguacacgagcgcgcug
cuggcggggacgaucacgagcggguggacguucggggcgggggcggcgcugcagaucccguucgcgaugcaga
uggcguaccgguucaacgggaucggggugacgcagaacgugcuguacgagaaccagaagcugaucgcgaacca
guucaacagcgcgaucgggaagauccaggacagccugagcagcacggcgagcgcgcuggggaagcugcaggac
guggugaaccagaacgcgcaggcgcugaacacgcuggugaagcagcugagcagcaacuucggggcgaucagca
gcgugcugaacgacauccugagccggcuggacaagguggaggcggaggugcagaucgaccggcugaucacggg
gcggcugcagagccugcagacguacgugacgcagcagcugauccgggcggcggagauccgggcgagcgcgaac
cuggcggcgacgaagaugagcgagugcgugcuggggcagagcaagcggguggacuucugcgggaagggguacc
accugaugagcuucccgcagagcgcgccgcacggggugguguuccugcacgugacguacgugccggcgcagga
gaagaacuucacgacggcgccggcgaucugccacgacgggaaggcgcacuucccgcgggaggggguguucgug
agcaacgggacgcacugguucgugacgcagcggaacuucuacgagccgcagaucaucacgacggacaacacgu
ucgugagcgggaacugcgacguggugaucgggaucgugaacaacacgguguacgacccgcugcagccggagcu
ggacagcuucaaggaggagcuggacaaguacuucaagaaccacacgagcccggacguggaccugggggacauc
agcgggaucaacgcgagcguggugaacauccagaaggagaucgaccggcugaacgagguggcgaagaaccuga
acgagagccugaucgaccugcaggagcuggggaaguacgagcaguacaucaaguggccgugguacaucuggcu
gggguucaucgcggggcugaucgcgaucgugauggugacgaucaugcugugcugcaugacgagcugcugcagc
ugccugaaggggugcugcagcugcgggagcugcugcaaguucgacgaggacgacagcgagccggugcugaagg
gggugaagcugcacuacacgugaagcucgcuuucuugcuguccaauuucuauuaaagguuccuuuguucccua
aguccaacuacuaaacugggggauauuaugaagggccuugagcaucuggauucugccuaauaagaaacauuua
uugucauugcaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaa
mRNA sequence encoding SARS-CoV2
spike protein (BspQI linearized
template), and including non-coding regions
(ITS, 5′ UTR, [symbol] [symbol] , 3′ UTR)
SEQ ID NO: 7
m7Gaggagaccaggaauuacauuugcuucugacacaacuguguucacuagcaaccucaaacaga [symbol] aug
uucguguuccuggugcugcugccgcuggugagcagccagugcgugaaccugacgacgcggacgcagcugccgc
cggcguacacgaacagcuucacgcgggggguguacuacccggacaagguguuccggagcagcgugcugcacag
cacgcaggaccuguuccugccguucuucagcaacgugacgugguuccacgcgauccacgugagcgggacgaac
gggacgaagcgguucgacaacccggugcugccguucaacgacgggguguacuucgcgagcacggagaagagua
acaucauccggggguggaucuucgggacgacgcuggacagcaagacgcagagccugcugaucgugaacaacgc
gacgaacguggugaucaaggugugcgaguuccaguucugcaacgacccguuccuggggguguacuaccacaag
aacaacaagagcuggauggagagcgaguuccggguguacagcagcgcgaacaacugcacguucgaguacguga
gccagccguuccugauggaccuggaggggaagcaggggaacuucaagaaccugcgggaguucguguucaagaa
caucgacggguacuucaagaucuacagcaagcacacgccgaucaaccuggugcgggaccugccgcagggguuc
agcgcgcuggagccgcugguggaccugccgaucgggaucaacaucacgcgguuccagacgcugcuggcgcugc
accggagcuaccugacgccgggggacagcagcagcggguggacggcgggggcggcggcguacuacguggggua
ccugcagccgcggacguuccugcugaaguacaacgagaacgggacgaucacggacgcgguggacugcgcgcug
gacccgcugagcgagaccaagugcacgcugaagaguuucacgguggagaaggggaucuaccagacgagcaacu
uccgggugcagccgacggagagcaucgugcgguucccgaacaucacgaaccugugcccguucggggagguguu
caacgcgacgcgguucgcgagcguguacgcguggaaccggaagcggaucagcaacugcguggcggacuacagc
gugcuguacaacagcgcgagcuucagcacguucaagugcuacggggugagcccgacgaagcugaacgaccugu
gcuucacgaacguguacgcggacagcuucgugauccggggggacgaggugcggcagaucgcgccggggcagac
ggggaagaucgcggacuacaacuacaagcugccggacgacuucacggggugcgugaucgcguggaacagcaac
aaccuggacagcaaggugggggggaacuacaacuaccuguaccggcuguuccggaagaguaaccugaagccgu
ucgagcgggacaucagcacggagaucuaccaggcggggagcacgccgugcaacgggguggagggguucaacug
cuacuucccgcugcagagcuacggguuccagccgacgaacggggugggguaccagccguaccgggugguggug
cugagcuucgagcugcugcacgcgccggcgacggugugcgggccgaagaagaguacgaaccuggugaagaaca
agugcgugaacuucaacuucaacgggcugacggggacgggggugcugacggagagcaacaagaaguuccugcc
guuccagcaguucgggcgggacaucgcggacacgacggacgcggugcgggacccgcagacgcuggagauccug
gacaucacgccgugcagcuucgggggggugagcgugaucacgccggggacgaacacgagcaaccagguggcgg
ugcuguaccaggacgugaacugcacggaggugccgguggcgauccacgcggaccagcugacgccgacguggcg
gguguacagcacggggagcaacguguuccagacgcgggcggggugccugaucggggcggagcacgugaacaac
agcuacgagugcgacaucccgaucggggcggggaucugcgcgagcuaccagacgcagacgaacagcccgcggc
gggcgcggagcguggcgagccagagcaucaucgcguacacgaugagccugggggcggagaacagcguggcgua
cagcaacaacagcaucgcgaucccgacgaacuucacgaucagcgugacgacggagauccugccggugagcaug
acgaagacgagcguggacugcacgauguacaucugcggggacagcacggagugcagcaaccugcugcugcagu
acgggagcuucugcacgcagcugaaccgggcgcugacggggaucgcgguggagcaggacaagaacacgcagga
gguguucgcgcaggugaagcagaucuacaagacgccgccgaucaaggacuucgggggguucaacuucagccag
auccugccggacccgagcaagccgagcaagcggagcuucaucgaggaccugcuguucaacaaggugacgcugg
cggacgcgggguucaucaagcaguacggggacugccugggggacaucgcggcgcgggaccugaucugcgcgca
gaaguucaacgggcugacggugcugccgccgcugcugacggacgagaugaucgcgcaguacacgagcgcgcug
cuggcggggacgaucacgagcggguggacguucggggcgggggcggcgcugcagaucccguucgcgaugcaga
uggcguaccgguucaacgggaucggggugacgcagaacgugcuguacgagaaccagaagcugaucgcgaacca
guucaacagcgcgaucgggaagauccaggacagccugagcagcacggcgagcgcgcuggggaagcugcaggac
guggugaaccagaacgcgcaggcgcugaacacgcuggugaagcagcugagcagcaacuucggggcgaucagca
gcgugcugaacgacauccugagccggcuggacaagguggaggcggaggugcagaucgaccggcugaucacggg
gcggcugcagagccugcagacguacgugacgcagcagcugauccgggcggcggagauccgggcgagcgcgaac
cuggcggcgacgaagaugagcgagugcgugcuggggcagagcaagcggguggacuucugcgggaagggguacc
accugaugagcuucccgcagagcgcgccgcacggggugguguuccugcacgugacguacgugccggcgcagga
gaagaacuucacgacggcgccggcgaucugccacgacgggaaggcgcacuucccgcgggaggggguguucgug
agcaacgggacgcacugguucgugacgcagcggaacuucuacgagccgcagaucaucacgacggacaacacgu
ucgugagcgggaacugcgacguggugaucgggaucgugaacaacacgguguacgacccgcugcagccggagcu
ggacagcuucaaggaggagcuggacaaguacuucaagaaccacacgagcccggacguggaccugggggacauc
agcgggaucaacgcgagcguggugaacauccagaaggagaucgaccggcugaacgagguggcgaagaaccuga
acgagagccugaucgaccugcaggagcuggggaaguacgagcaguacaucaaguggccgugguacaucuggcu
gggguucaucgcggggcugaucgcgaucgugauggugacgaucaugcugugcugcaugacgagcugcugcagc
ugccugaaggggugcugcagcugcgggagcugcugcaaguucgacgaggacgacagcgagccggugcugaagg
gggugaagcugcacuacacgugaagcucgcuuucuugcuguccaauuucuauuaaagguuccuuuguucccua
aguccaacuacuaaacugggggauauuaugaagggccuugagcaucuggauucugccuaauaagaaacauuua
uugucauugcaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaa
Initial Transcribed Sequence (ITS)
SEQ ID NO: 8
gggaga
Initial Transcribed Sequence (ITS)
SEQ ID NO: 9
aggaga
Initial Transcribed Sequence (ITS)
SEQ ID NO: 10
gggagaccaggaauu
Initial Transcribed Sequence (ITS)
SEQ ID NO: 11
aggagaccaggaauu
5′ untranslated region (UTR)
SEQ ID NO: 12
acauuugcuucugacacaacuguguucacuagcaaccucaaacaga
Kozak sequence
SEQ ID NO: 13
gccgcc
3′ untranslated region
SEQ ID NO: 14
agcucgcuuucuugcuguccaauuucuauuaaagguuccuuuguucccuaaguccaacuacuaaacuggggga
uauuaugaagggccuugagcaucuggauucugccuaauaagaaacauuuauugucauugc
3′ poly(A) sequence
SEQ ID NO: 15
aaaaaaaaaaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaa
ITS (SEQ ID: NO: 11) + HBG 5′ UTR (SEQ ID NO: 12) + Kozak
sequence (SEQ ID NO: 13)
SEQ ID NO: 17
aggagaccaggaauuacauuugcuucugacacaacuguguucacuagcaaccucaaacagagccgcc
5′HBA
SEQ ID NO: 18
agggcgaacuaguacucuucugguccccacagacucagagagaacgccacc
5′HSD
SEQ ID NO: 19
agggucccgcagucggcguccagcggcucugcuuguucgugugugugucguugcaggccuuauucgccgcc
5′ MRNA
SEQ ID NO: 20
agggaaauaagagagaaaagaagaguaagaagaaauauaagagccacc
5′NCA-7d
SEQ ID NO: 21
aggcaaaaaucaaaaucaaucaucaucacaacaucaacaaucaaucaucaacacaucaucaagacaccacc
3′ HBG
SEQ ID NO: 22
Agcucgcuuucuugcuguccaauuucuauuaaagguuccuuuguucccuaaguccaacuacuaaacuggggga
uauuaugaagggccuugagcaucuggauucugccuaauaagaaacauuuauugucauugc
3′ AES-mtRNRI
SEQ ID NO: 23
cugguacugcaugcacgcaaugcuagcugccccuuucccguccuggguaccccgagucucccccgaccucggg
ucccagguaugcucccaccuccaccugccccacucaccaccucugcuaguuccagacaccucccaagcacgca
gcaaugcagcucaaaacgcuuagccuagccacacccccacgggaaacagcagugauuaaccuuuagcaauaaa
cgaaaguuuaacuaagcuauacuaaccccaggguuggucaauuucgugccagccacacc
3′ ALB
SEQ ID NO: 24
Caucacauuuaaaagcaucucagccuaccaugagaauaagagaaagaaaaugaagaucaauagcuuauucauc
ucuuuuucuuuuucguugguguaaagccaacacccugucuaaaaaacauaaauuucuuuaaucauuuugccuc
uugucucugugcuccacucaccaaaaaauggaaagaaccu
3′ HBA
SEQ ID NO: 25
ugauaauaggcuggagccucgguggccaugcuucuugccccuugggccuccccccagccccuccuccccuucc
ugcacccguacccccguggucuuugaauaaagucuga
3′ S27a + R3U
SEQ ID NO: 26
uuguguaugcguuaauaaaaagaaggaacucguaaaaacucaauguauuucugaggaagcguggugcauaaug
ccacgcagcgucugcauaacuuuuauuauuucuuuuauuaaucaacaaa
5′ HBG-ITS
SEQ ID NO: 27
AGGAGACCAGGAAUUacauuugcuucugacacaacuguguucacuagcaaccucaaacagagccgcc
5′ HBG-ITS
SEQ ID NO: 28
GGGAGACCAGGAAUUacauuugcuucugacacaacuguguucacuagcaaccucaaacagagccgcc
5′ HBG
SEQ ID NO: 29
AGGacauuugcuucugacacaacuguguucacuagcaaccucaaacagagccgcc
5′ HBG
SEQ ID NO: 30
Gacauuugcuucugacacaacuguguucacuagcaaccucaaacagagccgcc
5′ NCA-7d-ITS
SEQ ID NO: 31
AGGAGACCAGGAAUUggcaaaaaucaaaaucaaucaucaucacaacaucaacaaucaaucaucaacacauc
aucaagacaccacc
5′ NCA-7d-ITS
SEQ ID NO: 32
GGGAGACCAGGAAUUggcaaaaaucaaaaucaaucaucaucacaacaucaacaaucaaucaucaacacauc
aucaagacaccacc
5′ NCA-7d
SEQ ID NO: 33
AGGcaaaaaucaaaaucaaucaucaucacaacaucaacaaucaaucaucaacacaucaucaagacaccacc
5′ NCA-7d
SEQ ID NO: 34
GGcaaaaaucaaaaucaaucaucaucacaacaucaacaaucaaucaucaacacaucaucaagacaccac
5′ NCA-7d
SEQ ID NO: 35
Gcaaaaaucaaaaucaaucaucaucacaacaucaacaaucaaucaucaacacaucaucaagacaccac

1. An mRNA encoding a SARS-CoV-2 spike protein, the mRNA comprising a 5′ UTR comprising an Initial Transcribed Sequence (ITS) of SEQ ID NO: 8 or SEQ ID NO: 9, optionally comprising one or more modified nucleobases.

2. The mRNA of claim 1, wherein the ITS comprises SEQ ID NO: 10 or SEQ ID NO: 11 or at least 8, or at least 10, or at least 12 consecutive nucleotides of SEQ ID NO: 10 or SEQ ID NO: 11, optionally comprising one or more modified bases.

3. The mRNA of claim 1, wherein the ITS does not contain any modified bases.

4. The mRNA of claim 1, wherein the ITS comprises modified uridine, which is optionally pseudouridine or Nl-methyl-pseudouridine.

5. The mRNA of claim 1, further comprising a 5′ cap.

6-10. (canceled)

11. The mRNA of claim 1, wherein the 5′ UTR further comprises a Kozak sequence, and wherein the mRNA further comprises a 3′ UTR and optionally a PolyA tract.

12. The mRNA of claim 11, wherein the 5′ UTR further comprises the nucleotide sequence substantially of SEQ ID NO: 12 or a derivative thereof.

13. The mRNA of claim 12, wherein the 5′ UTR comprises the nucleotide sequence of SEQ ID NO: 12, with uridine substituted with modified uridine, where the modified uridine is optionally pseudouridine or N1-methyl-pseudouridine.

14. (canceled)

15. The mRNA of claim 11, wherein the 3′ UTR substantially comprises the nucleotide sequence of SEQ ID NO: 14 or a derivative thereof; optionally with uridine substituted with modified uridine, where the modified uridine is optionally pseudouridine or Nl-methyl-pseudouridine.

16. The mRNA of claim 15, wherein the 3′ UTR substantially comprises the nucleotide sequence of SEQ ID NO: 15.

17. The mRNA of claim 11, comprising a poly(A) tract of about 100 nucleotides.

18. The mRNA of claim 1, wherein the spike protein has the amino acid sequence of SEQ ID NO: 5, optionally having from one to twenty-five amino acid substitutions.

19. The mRNA of claim 18, wherein the spike protein comprises one or more amino acid substitutions selected from L5F, P9L, S13I, L18F, T19R, T20N, P26S, A67V, HV69-70del, G75V, T76I, D80A, T95I, C136F, D138Y, G142D, Y144del, Y144S, Y145N, W152C, EF156-157del, R158G, R190S, E154K, R190S, D215G, LA242-243del, LAL242-244del, R246I, RSYLTPG246-252del, D253N, D253G, R346K, K417N, K417T, Y449H, L452R, L452Q, T478K, E484K, E484Q, F490S, N501Y, A570D, D614G, H655Y, Q677H, N679K, P681H, P681R, A701V, T716I, T859N, F888L, D950N, S982A, K986P, V987P, Q1071H, T1027I, D1118H, and V1176F.

20. (canceled)

21. The mRNA of claim 18, wherein the spike protein open reading frame has a nucleotide sequence substantially corresponding to SEQ ID NO: 2 or 4.

22. A method for synthesizing the mRNA of claim 1, comprising, contacting a linear DNA template encoding the mRNA under control of a promoter, with a RNA polymerase that recognizes said promoter, nucleotide triphosphate (NTP) reagents, and optionally a 5′ capping reagent.

23-32. (canceled)

33. An mRNA vaccine composition comprising the mRNA of claim 1, encapsulated in a lipid nanoparticle.

34. A method for preventing or reducing the probability of SARS-CoV-2 infection in a patient, comprising administering the mRNA vaccine of claim 33.

35-37. (canceled)

38. An mRNA encoding an antigen or therapeutic protein, the mRNA comprising a 5′ UTR, comprising an Initial Transcribed Sequence (ITS) of SEQ ID NO: 8 or SEQ ID NO: 9 or SEQ ID NO: 10 or SEQ ID NO: 11, the mRNA further comprising an open reading frame encoding the antigen or therapeutic protein, optionally comprising one or more modified nucleobases.

39. The mRNA of claim 38, wherein the ITS comprises SEQ ID NO: 10 or SEQ ID NO: 11 or at least 8, or at least 10, or at least 12 consecutive nucleotides of SEQ ID NO: 10 or SEQ ID NO: 11, optionally comprising one or more modified bases.

40. The mRNA of claim 38, wherein the ITS does not contain any modified bases.

41. The mRNA of claim 38, wherein the ITS comprises modified uridine, which is optionally pseudouridine or N1-methyl pseudouridine.

42. The mRNA of claim 38, further comprising a 5′ cap.

43-47. (canceled)

48. The mRNA of claim 38, wherein the 5′ Untranslated Region (UTR) comprises a Kozak sequence, and wherein the mRNA further comprises a 3′ UTR and optionally a PolyA tract.

49. The mRNA of claim 48, wherein the 5′ UTR further comprises the nucleotide sequence of SEQ ID NO: 12 or a derivative thereof.

50. The mRNA of claim 48, with uridine substituted with modified uridine, where the modified uridine is optionally pseudouridine or N1-methyl-pseudouridine.

51. The mRNA of claim 48, wherein the Kozak sequence has the sequence of SEQ ID NO: 13, optionally with one or more modified bases.

52. The mRNA of claim 48, wherein the 3′ UTR substantially comprises the nucleotide sequence of SEQ ID NO: 14.

53. The mRNA of claim 48, wherein the 3′ UTR comprises the nucleotide sequence of SEQ ID NO: 14, with uridine substituted with modified uridine, where the modified uridine is optionally pseudouridine or N1-methyl pseudouridine.

54. The mRNA of claim 48, comprising a poly(A) tract of about 100 nucleotides.

55. (canceled)

56. (canceled)

57. (canceled)

58. A method for synthesizing the mRNA of claim 38, comprising, contacting a linear DNA template encoding the mRNA under control of a promoter, with a RNA polymerase that recognizes said promoter and nucleotide triphosphate (NTP) reagents.

59. The method of claim 58, wherein the DNA template, the RNA polymerase, and NTP reagents are contacted in vitro.

60. The method of claim 58, wherein the DNA template, the RNA polymerase, and NTP reagents are contacted in a cell free system synthesizing the NTP reagents from precursors.

61. The method of claim 60, wherein the precursors comprise nucleotide monophosphate (NMP) reagents which are optionally prepared by depolymerizing cellular RNA.

62. The method of claim 58, wherein the RNA polymerase is T7 RNA polymerase.

63-68. (Canceled)

69. An mRNA composition comprising the mRNA of claim 38, and a transfection reagent or delivery vehicle.

70. The mRNA composition of claim 69, wherein the composition comprises a lipid nanoparticle delivery vehicle.

71. A method for expressing a therapeutic protein in a patient, comprising administering the mRNA composition of claim 69 to said patient.

72. (canceled)

73. (canceled)

74. An RNA encoding a protein of interest, the RNA comprising an Initial Transcribed Sequence (ITS) of SEQ ID NO: 10 or SEQ ID NO: 11.

75. The RNA of claim 74 wherein the ITS is followed by SEQ ID NO: 12, followed by a Kozak sequence of SEQ ID NO: 13, followed by an open reading frame encoding said protein.

76. The RNA of claim 75, comprising SEQ ID NO: 17, followed by an open reading frame encoding said protein.

77. The RNA of claim 75, wherein said open reading frame is followed by a 3′ UTR sequence.

78. The RNA of claim 77, wherein said 3′ UTR comprises SEQ ID NO: 14, followed by a poly(A) sequence.

79. The RNA of claim 74 wherein the RNA is an mRNA.

80-82. (canceled)

83. The RNA of any of claim 74 wherein the open reading frame encodes a SARS-CoV-2 spike protein.

84. The RNA of claim 83 wherein the RNA comprises a sequence selected from the group consisting of SEQ ID NO: 6 and/or SEQ ID NO: 7.

85. The RNA of claim 74 wherein the open reading frame encodes one or more influenza proteins.

86. The RNA of claim 74 wherein the open reading frame encodes a varicella proteins.
